SEQUENCE LISTING (14 sequences)

SEQ ID NO: 1 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 2 - protein, 48 aa, Homo sapiens
ANSEACRDGLRAVMECRNVTHLLQQELTEAQKGFQDVEAQAATCNHTV

SEQ ID NO: 3 - DNA, 58 bp, Artificial
  shRNA targeting human BST-2 mRNA
ccggcgcgatcttggtggtcctgttctcgagaacaggaccaccaagatcgcgtttttg

SEQ ID NO: 4 - DNA, 58 bp, Artificial
  shRNA targeting human BST-2 mRNA
ccgggcttgagaatgaagtcacgaactcgagttcgtgacttcattctcaagctttttg

SEQ ID NO: 5 - DNA, 57 bp, Artificial
  control for shRNA targeting human BST-2 mRNA
ccggcaacaagatgaagagcaccaactcgagttggtgctcttcatcttgttgttttt

SEQ ID NO: 6 - DNA, 23 bp, Artificial
  Forward primer for GAPDH cDNA
ccccttcattgacctcaactaca

SEQ ID NO: 7 - DNA, 20 bp, Artificial
  Reverse primer for GAPDH cDNA
cgctcctggaggatggtgat

SEQ ID NO: 8 - DNA, 20 bp, Artificial
  Forward primer for mouse BST-2 cDNA
tcaggagtccctggagaaga

SEQ ID NO: 9 - DNA, 20 bp, Artificial
  Reverse primer for mouse BST-2 cDNA
atggagctgccagagttcac

SEQ ID NO: 10 - DNA, 20 bp, Artificial
  Forward primer for BIM cDNA
atcggagacgagttcaacga

SEQ ID NO: 11 - DNA, 20 bp, Artificial
  Reverse primer for BIM cDNA
tgccttctccataccagacg

SEQ ID NO: 12 - DNA, 22 bp, Artificial
  Forward primer for Caspase-3 cDNA
caaaacctcagtggattcaaaa

SEQ ID NO: 13 - DNA, 22 bp, Artificial
  Reverse primer for Caspase-3 cDNA
cccatttcaggataatccattt

SEQ ID NO: 14 - protein, 53 aa, Homo sapiens
TIKANSEACRDGLRAVMECRNVTHLLQQELTEAQKGFQDVEAQAATCNHTVMA

(19) United States
(12) Patent Application Publication
(10) Pub. No.: US 2018/0200331 A1
(43) Pub. Date: Jul. 19, 2018

(54) BST-2 AS A THERAPEUTIC TARGET AND DIAGNOSTIC MARKER FOR BREAST CANCER GROWTH AND METASTASIS

(71) Applicant: University of Iowa Research Foundation, Iowa City, IA (US)

(72) Inventors: Chioma M. Okeoma, Iowa City, IA (US); Wadie D. Mahauad-Fernandez, Iowa City, IA (US)

(73) Assignee: University of Iowa Research Foundation, Iowa City, IA (US)

(21) Appl. No.: 15/743,324

(22) PCT Filed: Jul. 11, 2016
(86) PCT No.: PCT/US2016/041699

§ 371 Date: Jan. 10, 2018

Related U.S. Application Data
(60) Provisional application No. 62/191,105, filed on Jul. 10, 2015.

(51) Int. Cl. A61K 38/17; A61P 35/00

(52) U.S. Cl. CPC A61K 38/177; A61P 35/00

(57) ABSTRACT

Disclosed are compositions, kits, and methods for treating and/or diagnosing cancer in a subject in need thereof. The compositions, kits, and methods may be used to treat and/or diagnose cancers associated with BST-2 expression and/or BST-2 biological activity. Also disclosed are reagents that inhibit dimerization of BST-2 which may be administered as therapeutic agents for inhibiting BST-2 biological activity and treating cancers associated with BST-2 biological activity.

### CROSS-REFERENCE TO RELATED PATENT APPLICATIONS

The present application claims the benefit of priority under 35 U.S.C. § 119(e) to U.S. Provisional Patent Application No. 62/191,105, filed on Jul. 10, 2016, the content of which is incorporated herein by reference in its entirety.

### STATEMENT REGARDING FEDERALLY SPONSORED RESEARCH OR DEVELOPMENT

This invention was made with government support under grant number P30 CA086862 awarded by the National Institutes of Health. The government has certain rights in the invention.

### BACKGROUND

The field of the invention relates to compositions, kits, and methods for treating and/or diagnosing cancer in a subject. In particular, the compositions, kits, and methods relate to treating and/or diagnosing cancer in a subject having or at risk for developing a cancer associated with BST-2 activity, such as breast cancer.

BST-2 is a transmembrane protein that has been recognized for its antiviral activity. Recent studies have also shown that BST-2 plays a role in the development and progression of cancers. (See Mahauad-Fernandez and Okeoma, “BST-2: at the crossroads of vrial pathogenesis and oncogenesis,” Future Virol. 10.221/fvl.15.113 (2016); and Mahauad-Fernandez and Okeoma, “The role of BST-2/Tetherin in host protection and disease manifestation,” Immun. Inflamm. Dis. 2016 March; 4(1): 4-23; the contents of which are incorporated herein by reference in their entireties).

The present inventors have found that suppression of BST-2 expression or inhibition of BST-2 dimerization is necessary for the antiviral and oncogenic properties of BST-2 and have consequently identified possible targets within the BST-2 molecular structure that can be exploited for therapeutic value. Furthermore, we have identified that disruption of BST-2 dimerization with peptides that bind to the BST-2 ectodomain controls cancer cell behavior and tumor growth. For these discoveries, we are seeking intellectual property protection.

BST-2:BST-2 form a potent signaling pair in breast tumor cells. Signals from dimerized BST-2 impact different downstream cellular behaviors, including cancer cell to extracellular matrix interaction, cancer cell to cancer cell interaction, cancer cell to stromal cell interaction, adhesion, anchorage independent growth of cancer cells, resistance to anoikis, decreased apoptosis, cellular migration, invasion, and metastatic spread of cancer cells. Previous findings reveal that BST-2 expression in breast cancer cells promotes breast tumor growth and metastasis in humans and in our mouse model of breast cancer and that BST-2 DNA is hypomethylated in breast cancer cells indicating that there is an epigenetic component to protein expression (Mahauad-Fernandez W D et al. “Bone Marrow Stromal Antigen 2 (BST-2) DNA Is Demethylated in Breast Tumors and Breast Cancer Cells.” PLoS One. 2015; 10(4):e0123931. Epub 2015/04/11; Mahauad-Fernandez W D et al., “Bone marrow stromal antigen 2 expressed in cancer cells promotes mammary tumor growth and metastasis.” Breast Cancer Research: BCR. 2014; 16(6):493. Epub 2014/12/17; the contents of which are incorporated herein by reference in their entireties).

Increasing experimental evidence demonstrates that the ability of BST-2 to form dimers plays a key role in BST-2-mediated breast tumorigenesis and emerging preclinical data suggest that blockade of BST-2 dimerization may be therapeutically important in BST-2-amplified breast cancer. Consequently, the present inventors have demonstrated that peptides directed at disruption of BST-2 dimers inhibited cancer cell adhesion and reduced tumor burden in a mouse model of breast cancer. These data emphasize the crucial role that BST-2 dimerization may play in BST-2-driven breast cancers and identify BST-2 as a druggable target for the treatment of breast cancer. The inventors' findings could be applicable to other cancers in which BST-2 is upregulated and to virus-induced cancers. (See Mahauad-Fernandez W D et al. “Bone Marrow Stromal Antigen 2 (BST-2) DNA Is Demethylated in Breast Tumors and Breast Cancer Cells.” PLoS One. 2015; 10(4):e0123931. Epub 2015/04/11; Jones P H et al., “BST-2/tetherin is overexpressed in mammary gland and tumor tissues in MMTV-induced mammary cancer.” Virology. 2013 September; 444(1-2):124-39, Epub 2013 Jun. 25, the contents of which is incorporated herein by reference in its entirety).

### SUMMARY

Disclosed are compositions, kits, and methods for treating and/or diagnosing cancer in a subject in need thereof. In particular, the compositions, kits, and methods may be used to treat and/or diagnose cancers associated with BST-2 expression and/or BST-2 biological activity.

The present inventors have determined that bone marrow stromal antigen 2 (BST-2), a known antiviral protein, is implicated in invasiveness of breast cancer cells and formation of metastasis in mouse models of breast cancer. The present inventors have determined that BST-2 dimerization is implicated in progression of breast cancer, which suggests that inhibition of BST-2-dimerization provides a rationale for targeted therapy in breast cancer patients. Accordingly, the presently disclosed methods include methods for treating cancer in a subject in need thereof, wherein the cancer is associated with BST-2 expression or biologica activity and the method comprising administering a therapeutic agent that inhibits the expression or the biological activity of BST-2. Suitable cancers treated by the methods include breast cancer such as aggressive and/or metastatic breast cancers and triple negative breast cancer.

Therapeutic agents that are administered in the disclosed methods may include therapeutic agents that inhibit the expression of BST-2, for example, via RNA interference using small hairpin RNA (shRNAs), small interfering RNAs (siRNAs), microRNAs (miRNAs), and/or PIWI-interacting RNAs (piRNAs). In addition, therapeutic agents that are administered in the disclosed methods also may include therapeutic agents that inhibit the biological activity of BST-2.

In some embodiments of the disclosed methods, the therapeutic agent that is administered is a therapeutic agent that inhibits dimerization of BST-2. Suitable therapeutic agents that inhibit dimerization of BST-2 may include peptides. Suitable peptides for the disclosed methods comprise a contiguous amino sequence of BST-2 of at least about 5, 10, 20, 30, 40, 50, 60, 70, 80, 90, or 100 amino acids, for example, the peptide comprises a contiguous amino acid sequence from amino acid 47 to amino acid 95 which may act as a decoy to prevent dimerization of full-length BST-2. Peptides that are administered in the disclosed methods may be conjugated to a reagent that facilitates cell penetration (e.g., penetratin, TAT, low molecular weight protamine, and poly(arginine)₈) or loaded into biologicals that augment peptide penentration and delivery such as nanoparticles, extracellular vesicles (exosomes, ectosomes, microvesicles, microparticles, apoptotic bodies).

Also disclosed are pharmaceutical compositions. The disclosed pharmaceutical compositions may include a therapeutic agent that inhibits expression or biological activity of BST-2 and a carrier. For example, the contemplated pharmaceutical compositions may comprise a peptide as disclosed for use in the above-described methods and a carrier.

Also contemplated herein are methods for diagnosing aggressive and/or metastatic breast cancer in a subject in need thereof. The diagnostic methods may include detecting expression or biological activity of BST-2 and may utilize one or more reagents for detecting expression or biological activity of BST-2 (e.g., polynucleotide reagents, antibodies, and the like). The diagnostic methods further may include detecting methylation or the absence of methylation of the BST-2 promoter. The diagnostic methods further may include detecting BST-2 or biological activity of BST-2 in cells, bodily fluids, or extracellular vesicles including cells, bodily fluids, or extracellular vesicles (exosomes, ectosomes, microvesicles, microparticles, apoptotic bodies) derived from cancer cells or other cells.

### BRIEF DESCRIPTION OF THE FIGURES

FIG. 1. BST-2 mRNA is prevalent in highly aggressive tumors and associates with patients' poor survival: (A) RNA-seq data (n=100) of paired tumor versus normal breast tissues from TCGA (BRCA) data portal presented as scatter plot and heat map show that BST-2 is significantly elevated in tumor tissues compared to matched normal breast tissues. (B) Levels of BST-2 in tumor tissues of patients bearing different subtypes of invasive breast carcinomas show that BST-2 is upregulated in different breast tumors subtypes with the exception of the basal subtype. (C) BST-2 expression in tumors from Uppsala (Sweden) breast cancer patients obtained from GSE4922 was segregated into three BST-2 expression levels (relative units): Low=6.0-7.5, Intermediate=7.5-9.0, and High=9.0-11.0. (D) Tumor size in patients with low, intermediate, or high levels of BST-2 is shown. (E) BST-2 GC-RMA signal scores from healthy, ductal carcinoma in situ (DCIS), and invasive ductal carcinoma (IDC) tumor bearing patients obtained from GSE21422. (F) BST-2 levels from normal, primary tumors (tumors), and metastatic tumors (metastatic) of patients bearing invasive breast cancer (TCGA). (G) Kaplan Meier survival analysis using TCGA (BRCA) primary tumor samples segregated into high and low BST-2 levels show a significant link between low BST-2 and patient survival. The median overall survival (OS) time and the area under the curve (AUC) for each group are shown. (H) Mammary epithelial and stromal cells obtained from normal and invasive breast cancer patients (GSE10797) show elevated BST-2 expression in cancerous epithelial cells but not in cancerous stromal cells. In all panels, numbers correspond to p-values. The relative units for BST-2 RNA levels acquired from TCGA and GEO datasets are SEM-normalized and Centralized Log 2(x+1). Error bars represent standard deviations and significance was taken at P<0.01**. ns=not significant.

FIG. 2. Suppression of BST-2 in cancer cells increases tumor latency and decreases tumor mass in vivo: (A) Knockdown of endogenous BST-2 expression in 4T1 cells increases tumor latency computed as (number of tumor-free injected mice/number of injected mice)×100. (B) Tumor volume (TV) computed as TV=0.5(Length*Width²) over time is significantly reduced when BST-2 is suppressed in 4T1 cells. (C) Tumor cells tracked in vivo with IVIS imaging system show significant reduction in luciferase expression in BST-2-suppressed sh413 compared to BST-2-expressing shControl injected mice. (D) Loss of BST-2 in cancer cells reduced tumor mass. Tumor weight (numbers, g) and gross images obtained at necropsy are shown. All mice implanted with 4T1 shControl or sh413 cells developed mammary tumors with variation in size. Numbers represent Average±SEM. Scale bar=5 mm.

FIG. 3. Down-modulation of BST-2 in cancer cells reduces mammary cancer metastases: (A) Representative images of tumor cells tracked in vivo with IVIS imaging at different time points. Images show higher luciferase bioluminescence in 4T1 shControl injected mice compared to sh413 injected mice. (B) Representative luciferase bioluminescence accompanied with abdominal and gastrointestinal tract (GI tract) gross images of uninjected (upper panel), shControl-implanted (middle panel), and sh413-implanted mice (lower panel). Arrow heads point to mammary tumors (middle column) and intestinal/mesenteric tumors (right column). Scale bar=5 mm. (C) Number of secondary tumors in intestine/mesentery plotted as average of all mice. (D) Representative intestine/mesentery histology images from 4T1 shControl and sh413 injected mice confirming increased mesenteric tumors (arrows) in shControl mice compared to sh413 injected mice. A mesenteric lymph node is demarcated by an asterisk (not to be confused with a tumor mass). (E) Representative gross liver images of 4T1 shControl and sh413 injected mice. Arrows are pointing to tumors. (F) Representative gross images of lungs showing visible pulmonary nodules (arrows) in shControl-implanted mice. (G) Percent incidence of liver and lung metastases. (H) Lung histology from shControl (upper left) and sh413 (upper right) injected mice. Lung from the 4T1 shControl mice had multiple large tumors (tumors demonstrated by asterisk) and marked infiltration of the alveolar septa and alveolar spaces by neutrophils (yellow arrows). Boxed regions are shown at higher magnification (40×) for shControl (lower left) and sh413 (lower right). Error bars represent standard deviations and significance was taken at P<0.01**.

FIG. 4. BST-2 expression in cancer cells is a strong prognostic factor for morbidity and overall survival: (A) Clinical score plot of mice implanted with 4T1 BST-2-expressing shControl and BST-2-suppressed sh413 cells. Clinical signs were scored as follows: 0=no abnormal clinical signs; 1=ruffled fur but lively; 2=ruffled fur, activity level slowing, sick; 3=ruffled fur, eyes squeezed shut, hunched, hardly moving, very sick; 4=moribund; 5=dead [23]. (B) Representative gross images of the abdomen of uninjected (left), shControl-implanted (middle), and sh413-implanted (right) mice. Arrow points to metastatic ascites (middle panel). (C) Representative splenic gross images (top panel insets) and spleen histology at low magnification (4×, top panel). Boxed regions are shown at higher magnification (60×) from uninjected (left panel), shControl (middle panel), and sh413 (right panel) injected BALB/c mice (bottom panels). There was marked expansion of red pulp due to granulocytic hyperplasia in the shControl spleen with slightly increased number of granulocytes in the red pulp of the sh413 spleen. Scale bar=5 mm. (D) Kaplan Meier survival plot of mice implanted with BST-2-expressing shControl and BST-2-suppressed sh413 4T1 cells. Numbers are p values and error bars represent standard deviations. Median overall survival (OS) time and the area under the curve (AUC) are shown for each group.

FIG. 5. Elevated mammary tumor cell BST-2 regulates cancer cell adhesion. PKH67Green-labeled BST-2-expressing shControl and BST-2-suppressed sh137 and sh413 E0771 cells were allowed to adhere to murine embryonic fibroblasts (MEF). (A) Representative confocal images of adhered cells and (B) image J quantification of PKH67Green+ cells per field (n=5) are shown. Images were taken at 20×. (C) Analysis of cancer cell adhesion to MEF by quantification of luciferase bioluminescence shControl and sh413 cells. Luciferase assay quantified expression represented as relative light units (RLU) and images (inset) were taken with the IVIS 3D optical imaging system and analyzed with Living Image Software. (D) Representative images and (E) Quantification of shControl and sh413 4T1 cells adhered to collagen-coated plates. (F) Representative images and (G) Quantification of shControl and sh413 4T1 cells adhered to fibronectin-coated plates. Images were taken at 10×. Experiments were performed multiple times with similar results. Error bars represent standard deviations and significance was taken at P<0.05*, <0.01**. ns=not significant.

FIG. 6. Suppression of BST-2 expression in cancer cells results in anchorage-independency: (A and C) Quantification of the number of colonies formed in soft agar by BST-2-expressing shControl, BST-2-suppressed sh137 and sh413 murine cells, MCF-7 (positive control), HMLE and NMuMG cells following a 20-day transformation assay. The bar represents number of colonies per field (n=5) for each cell type. Error bars correspond to standard deviations. Significance was taken at P<0.001**. (B and D) Representative images of crystal violet stained colonies from a soft agar assay showing anchorage-independent growth of cancer cells. Clones were imaged at 4×. Experiments were repeated multiple times with similar results. ns=not significant.

FIG. 7. Endogenous BST-2 expressed in mammary cancer cells controls cell migration and invasion: (A) Representative images of cell migration performed by scratch assay. Suppression of BST-2 expression by sh137 and sh413 reduced rate of cancer cell migration into the scratch wound. (B) Quantification of cells that migrated into the scratch wound at 0, 6, and 24 h. (C) Migration assay by Boyden chamber. Representative images taken at 4×. (D) Migrated cells were imaged and the number of migrated cells counted with image J. Similar results were obtained with 4T1 shControl and sh413 cells. (E) BST-2-expressing shControl and BST-2 suppressed sh137 and sh413 E0771 cells and (F) shControl and sh413 4T1 cells were plated in low-serum medium on Matrigel coated cell inserts and allowed to migrate for 24 h. Cells were stained with Giemsa stain. Representative images taken at 20× are shown. (G) Quantification of cells that invaded through Matrigel. Cells from 5 different fields were counted and averaged. Error bars corresponds to standard deviations. Significance was taken at P<0.001** and P<0.05*. Experiments were repeated multiple times with similar results.

FIG. 8. Schematic portraying the role of cancer cell intrinsic BST-2 in mammary cancer development.

FIG. 9. BST-2 expression in human breast cancer cell lines. (A) Expression of BST-2 mRNA from normal mammary epithelial cells (HMLE), luminal A MCF-7 tumorigenic cells, and claudin-low MDA-MB-231 tumorigenic cells as determined by RT-qPCR. (B) BST-2 surface expression from HMLE, MCF-7 and MDA-MB-231 cells as determined by flow cytometry. Numbers in parenthesis correspond to BST-2 expression presented as a percentage. All RT-qPCR data are normalized to GAPDH and presented as fold change over HMLE. Error bars represent standard deviations and significance was taken at P<0.01**. Experiments were repeated multiple times with similar results.

FIG. 10. BST-2 expressed in mammary cancer cells is suppressed by BST-2-targeting shRNAs: (A) Expression of BST-2 mRNA is higher in murine mammary tumor tissues and cells (E0771luc and 4T1luc) compared to normal mammary gland tissues as determined by RT-qPCR. Following stable transduction of E0771luc and 4T1luc cells with lentiviruses expressing different BST-2 targeting (sh137 and sh413) and non-targeting (shControl) shRNA, levels of BST-2 (B and C) mRNA expression were measured by real-time quantitative PCR, (D) surface protein expression was measured by flow cytometry (FACS) and (E) total BST-2 protein was measured by Western blot. Numbers correspond to band quantifications. Percent (%) gene expression is calculated as BST-2/GAPDH*100. All RT-qPCR data are normalized to GAPDH and presented as fold change over Normal tissue or shControl cells. Error bars represent standard deviations and significance was taken at P<0.01**.

FIG. 11. BST-2 downregulation decreases E0771 cell dissemination and growth in vivo. (A) Knockdown of endogenous BST-2 expression in E0771 cells increases tumor latency. (B) Representative images of tumor cells tracked in vivo with IVIS imaging system at different time points. Images show higher luciferase bioluminescence in shControl E0771 injected mice compared to sh413 injected mice. (C) Representative Luciferase bioluminescence accompanied with abdominal and gastrointestinal tract (GI tract) gross images of uninjected (upper panel), shControl-implanted (middle panel), and sh413-implanted mice (lower panel). Arrow heads point to GI tumors. (D) Number of secondary tumors in intestine/mesentery plotted as average of all mice. (E) Percent incidence of liver and lung metastases. Error bars represent standard deviations and significance was taken at P<0.01**.

FIG. 12. BST-2 expression in cancer cells predicts host survival: (A) Clinical score plot of mice implanted with BST-2-expressing E0771 shControl and BST-2-suppressed sh413 cells. Clinical signs were scored as follows: 0=no abnormal clinical signs; 1=ruffled fur but lively; 2=ruffled fur, activity level slowing, sick; 3=ruffled fur, eyes squeezed shut, hunched, hardly moving, very sick; 4=moribund; 5=dead [23]. (B) Representative images of the abdomen and feet of uninjected, shControl, and sh413 C57BL/6 mice implanted with E0771 cells. Arrow points to metastatic ascites (upper-middle panels) and shock (lower-middle panel). (C) Kaplan Meier survival plot of mice implanted with BST-2-expressing shControl and BST-2-suppressed sh413 E0771 cells. Number corresponds to p value. Error bars represent standard deviations. Median overall survival (OS) time and the area under the curve (AUC) for each group are shown.

FIG. 13. BST-2 overexpression enhances anchorage-independency, cancer cell migration, and invasion: (A) Expression of BST-2 mRNA from MCF-7 cells stably transfected with an empty plasmid (Vector) or with a BST-2-expressing plasmid (WT BST-2) as determined by RT-qPCR. (B) Representative images of colonies from a soft agar assay showing anchorage-independent growth of MCF-7 cells. Clones were imaged at 10×. (C) Vector-expressing MCF-7 cells form smaller colonies compared to BST-2-expressing MCF-7 cells. Data is presented as percent normalized to Vector-expressing cells. (D) Representative images of cell migration by vector and WT BST-2 expressing cells and image J quantification of migration events (bars). (E) BST-2-expressing and Vector-expressing MCF-7 cells were plated in Matrigel coated cell inserts and allowed to invade for 24 h. Cells were stained with Giemsa stain. Representative images taken at 20× and image J quantification of invasion events (bars) are shown. Error bars corresponds to standard deviations. Significance was taken at P<0.001** and P<0.05*. ns=not significant.

FIG. 14. Endogenous BST-2 has no effect on proliferation of mammary cancer cells: (A and B) BrdU incorporation assay performed on shControl, sh137, and sh413 E0771 and 4T1 cells respectively. Absorbance was measured at 450 nm using a Tecan Infinite M200 Pro plate reader or cells were imaged using a Zeiss 710 confocal microscope (only for E0771 cells). Images were processed using Image J software. (C and D) MTT metabolism assay performed on shControl, sh137, and sh413 E0771 and 4T1 cells to determine cell viability. Absorbance was read at 590 nm using a Tecan Infinite M200 Pro plate reader. Results are expressed as the means±standard deviations of optical density (OD). BrdU (green), BST-2 (red), and DAPI (blue). Numbers correspond to p values. Error bars represent standard deviations. Experiments were repeated multiple times with similar results.

FIG. 15. Various cancer types including breast cancer have different levels of BST-2 expression. (A) to (C) BST-2 transcript levels in paired normal (N) and tumor (T) tissues of patients bearing different cancer types. Data analyzed are from TCGA. Lung adenocarcinoma=Lung A. C., Lung Squamous Cell Carcinoma=Lung S. C. C., Kidney Papillary Cell Carcinoma=Kidney P. C. C., Kidney Clear Cell Carcinoma=Kidney C. C. C. (D) BST-2 mRNA levels of normal, primary tumor, and metastatic tumor of invasive breast cancer bearing patients from TCGA repository. (E) BST-2 mRNA levels in different breast cancer subtypes. BST-2 levels in all tumor subtypes are compared to the BST-2 level in normal tissues. Data does not include metastatic tumors. (F) to (I) Box plots of BST-2 transcript levels in stages I, II, III, and IV of (F) luminal A, (G) luminal B, (H) HER2-enriched, and (I) basal breast cancers. Data does not include metastatic tumors. (J) BST-2 mRNA levels in different breast cancer cell lines. BST-2 levels in all cancer cell lines are compared to BST-2 levels in normal HMLE cells and normalized to GAPDH. (K) BST-2 surface expression from HMLE, MCF-7, BT-474, SK-BR-3 and MDA-MB-231 cells as determined by flow cytometry. Numbers in parenthesis correspond to BST-2 expression presented as a percentage. Significance was taken at P<0.05 (*), P<0.01 (**) and P<0.001 (****). Error bars correspond to standard error of the mean (SEM) and n.s=not significant.

FIG. 16. BST-2 DNA in breast tumors is hypomethylated compared to normal tissues. (A) BST-2 expression versus methylation plot among all primary tumor (red) and normal (black) breast tissues from BRCA in the TCGA. Beta-value ranges from 0 to 1. Beta-values closer to one depict hypermethylation and closer to 0 depict hypomethylation. (B) Methylation status among different CpG sites in primary tumor (red) and normal (black) breast tissues from invasive breast cancer bearing patients (TCGA). Significance was taken at P<0.05 (*) and P<0.001 (****). Error bars correspond to SEM.

FIG. 17. Methylation drives BST-2 expression in breast tumors, but not in normal breast tissue. (A) Location of the nine probes included in the Human Methylation 450 array that are associated with the BST-2 gene. Introns are solid gray rectangles while exons are solid black rectangles. (B) and (C) Correlation analysis between BST-2 mRNA levels and methylation values of probes 1 to 9 in (B) primary tumors and (C) normal breast tissues from TCGA. Best-fit line and r² values are shown for each probe.

FIG. 18. BST-2 DNA methylation pattern in different breast tumor subtypes. (A) Methylation status among different CpG sites from different breast cancer subtypes and normal breast tissue. Data does not include metastatic tumors. (B) to (J) Box plots of methylation values among different breast cancer subtypes and normal tissues corresponding to (B) probe 1, (C) probe 2, (D) probe 3, (E) probe 4, (F) probe 5, (G) probe 6, (H) probe 7, (I) probe 8, (J) probe 9. For statistical analysis, each subtype was compared to the normal breast tissue. The luminal subtypes were compared to each other. Significance was taken at P<0.05 (*), P<0.003 (**), P<0.0005 (***), and P<0.0001 (****). Error bars correspond to 95% CI.

FIG. 19. BST-2 expression inversely correlates with methylation density in neoplastic breast epithelial cell lines. (A) BST-2 and APOBEC3G (A3G) transcript levels in normal and tumor epithelial cells from patients with breast cancer. (B) BST-2 and A3G transcripts present in normal and tumor stromal cells of patients with breast cancer. Data were downloaded from GEO dataset GSE10797. (C) BST-2 expression levels among two luminal A cell lines (MCF-7 and T47D) and two triple-negative cell lines (MDA-MB-231 and SUM-159). Data were acquired from GEO dataset GSE41313 (bar graph, Log 2 units) and GEO dataset GSE45732 (gray line graph, RPKM units). (D) Methylation status among different CpG sites from the breast cancer cell lines whose mRNA is presented in line graph in 5C. Data acquired from GEO dataset GSE49794. (E) BST-2 expression versus methylation plot among the four breast cancer cell lines presented in 5C (line graph) and 5D. Methylation beta-value is an average of probes 3 to 9±SEM. Methylation data was from GSE49794 (beta-value) and expression data was from GSE45732, a superseries from the same cells. Significance was taken at P<0.05 (*). n.s=not significant. Error bars correspond to standard error of the mean (SEM).

FIG. 20. The effect of demethylating agents on BST-2 expression. (A) Meta-analysis of BST-2, Claudin-6 (CLDN6), and GAPDH expression levels in HB2, SK-BR-3, MDA-MB-231 cells (downloaded from GEO dataset GSE28976) and MCF-7 cells (downloaded from GEO dataset GSE36683) following treatment with 5-aza-2′-deoxycytidine (DAC). MCF-7 data was acquired from a different dataset, hence the difference in expression values. (B) BST-2, Claudin-6 (CLDN6), and GAPDH expression levels in HMLE, MCF-7, SK-BR-3, and MDA-MB-231 cells following treatment with DMSO (vehicle) or 1 uM of 5-azacytidine (5-AzaC) for 5 days. (C) BST-2 surface expression from HMLE, MCF-7, SK-BR-3 and MDA-MB-231 cells treated with DMSO (vehicle) or 1 uM of 5-azacytidine (5-AzaC) for 5 days as determined by flow cytometry. Numbers in parenthesis correspond to BST-2 expression presented as a percentage. Significance was taken at P<0.05 (*). Error bars correspond to SEM. n.s=not significant.

FIG. 21. Covalent dimerization of BST-2 mediates adhesion of cancer cells to ECM proteins and other cells. (A) Representative bright field and confocal images of fibronectin adhered cells and (B & C) quantification of MCF-7 cells expressing Vector, WT or C3A BST-2 adhered to (B) fibronectin- or (C) collagen-coated plates as relative fluorescence intensity. (D-F) PKH67Green-labeled MCF-7 cells expressing Vector, WT or C3A BST-2 were allowed to adhere to MCF-7 Vector (D, left) or to MCF-7 WT (D, right) monolayers, (E) to HUVEC monolayers or (F) to macrophage monolayers. Each bar corresponds to six different wells. Error bar corresponds to SEM. Significance was taken at P<0.05*, P<0.01** and P<0.001***. n.s=not significant.

FIG. 22. Recombinant extracellular domain (ECD) of BST-2 binds to BST-2 in vitro and prevents BST-2-mediated adhesion of breast cancer cells. PKH67Green-labeled shControl (shCTL)- or sh413 (shBST-2)-expressing (A) 4T1 or (B) E0771 cells were allowed to adhere to plates that were coated with 0, 20 or 50 ng of recombinant BST-2 (rBST-2) and fluorescence assessed with a plate reader. (C) Representative confocal images of cells attached to wells coated with increasing concentrations of rBST-2. (D) Quantification of PKH67Green-labeled MCF-7 cells expressing Vector, WT or C3A BST-2 adhered to plates that were coated with 0, 20 or 50 ng of rBST-2. (E) PKH67Green-labeled MCF-7 cells expressing Vector, WT or C3A BST-2 were allowed to adhere to MCF-7 WT monolayers that were previously blocked with rBST-2 or PBS (Vehicle). Cells that adhered to WT MCF-7 monolayers were quantified and represented as relative fluorescence intensity. Each data point corresponds to six different wells analyzed. Error bars correspond to SEM. Significance was taken at P<0.05* and P<0.01**. n.s=not significant.

FIG. 23. BST-2 in cancer cells mediates cancer cell adhesion to each other, resulting in cancer cell clustering. (A) Representative confocal images of 4T1 cells attached to monolayers of shControl (left) or sh413 (right) 4T1 cells. (B) Quantification of PKH67Green-labeled shControl- (shCtl) and shBST-2-expressing (sh413) cells that adhered to 4T1 shCtl (left) and sh413 (right) monolayers for 4 hours. Each data point corresponds to six different wells analyzed. Error bars correspond to SEM. Significance was taken at P<0.05* and P<0.01**.

FIG. 24. Covalent dimerization of BST-2 is important for anchorage-independent growth of breast cancer cells. (A, C and E) Representative images of crystal violet-stained colonies from a soft agar assay showing anchorage-independent growth of (A) 4T1 cells expressing shControl (shCTL) or sh413 cosntructs; (C) 4T1 sh413 cells in which WT hBST-2 (sh413 WT) or C3A hBST-2 (sh413 C3A) was rescued; or (E) MCF-7 cells expressing Vector, WT or C3A BST-2. Clones were imaged at 10×. (B, D and F) The diameter of colonies from 5 different fields were measured, averaged and a percent calculated relative to either (B) shControl for 4T1 cells, (D) 4T1 sh413 WT cells or (F) Vector for MCF-7 cells which was set up to 100% following a 35-day transformation assay. Error bars correspond to SEM. Significance was taken at P<0.001***, P<0.001** and P<0.05*. n.s=not significant.

FIG. 25. BST-2 dimerization renders cancer cells resistance to anoikis via downregulation of BIM. (A) Quantification of Annexin V+; Annexin V+, 7-AAD+; 7-AAD+ or double negative cells to determine cells undergoing aopotosis. (B & C) RNA levels of BST-2, the apoptotic factors BIM and Caspase-3 were analyzed by RT-qPCR normalizing with GAPDH in cells plated on (B) Vehicle or (C) Poly-HEMA. (D) Cleaved Caspase-3 (active form) and BIM protein levels were analyzed by western blot using GAPDH as a loading control. Error bars corresponds to standard deviations for RNA data (B & C). Significance was taken at P<0.05* and P<0.01**. n.s=not significant.

FIG. 26. BST-2 dimerization renders cancer cells resistance to anoikis via downregulation of BIM. MCF-7 cells expressing Vector, WT, or C3A BST-2 were cultured onto plates that were previously coated with 95% Ethanol (Vehicle) or Poly-HEMA and incubated at 37° C. for 48 hours. Cells were collected to do viability assays using (A) trypan blue staining or (B) a MTT assay reading absorbance at 590 nm. Moreover, cells plated on Vehicle or Poly-HEMA were collected and pelleted to isolate RNA and protein. (C & D) RNA levels of BST-2 and the apoptotic factor BIM were analyzed by RT-qPCR normalizing with GAPDH in cells plated on (C) Vehicle or (D) Poly-HEMA. (E) Cleaved Caspase-3 (active form) and BIM protein levels were analyzed by western blot using GAPDH as a loading control. Numbers correspond to band quantifications (relative units). Error bars corresponds to SEM for viability assays (A & B) and to standard deviations for RNA data (C & D). Significance was taken at P<0.05* and P<0.01**. n.s=not significant.

FIG. 27. Breast cancer cells that cannot form BST-2 dimers are deficient in breast tumor formation. (A) Representative images and (B) luciferase activity quantification of tumor cells tracked in vivo with IVIS imaging at different time points form mice injected with sh413 WT (n=8) or sh413 C3A (n=8) 4T1 cells. (C) Tumor volume (TV) computed as TV=0.5 (length*width2) over time in mice injected with sh413 WT or sh413 C3A 4T1 cells. (D) Clinical score plot of mice implanted with 4T1 BST-2-expressing shControl and BST-2-suppressed sh413 cells. Clinical signs were scored as described in FIG. 4. (E) Weight of primary tumors from mice injected with sh413 WT or sh413 C3A 4T1 cells. (F-H) Number of metastatic tumors found in the (F) lung, (G) liver and (H) mesentery of mice injected with sh413 WT or sh413 C3A 4T1 cells. Error bars corresponds to SEM. Significance was taken at P<0.05*. n.s=not significant.

FIG. 28. Breast cancer cells that cannot form BST-2 dimers are deficient in breast tumor formation. (A) Representative images and (B) luciferase activity quantification of tumor cells tracked in vivo with IVIS imaging at different time points form mice injected with shControl (n=3), sh413 (n=8), sh413 WT (n=8) or sh413 C3A (n=8) 4T1 cells. (C) Tumor volume (TV) computed as TV=0.5 (length*width2) over time in mice injected with shControl, sh413, sh413 WT or sh413 C3A 4T1 cells. All mice implanted with 4T1 shControl, sh413 or sh413 WT cells developed mammary tumors with variation in size. However, only 3 out of 8 sh413 C3A-injected mice developed tumors.

FIG. 29. Breast cancer cells that cannot form BST-2 dimers have decreased metastatic capacity resulting in increased host survival. (A & B) Primary tumor (A) weight and (B) volume determined from mice injected with shControl, sh413, sh413 WT, or sh413 C3A 4T1 cells at the experimental end-point. (C) Number of metastatic tumors found in the (C) lung, liver and mesentery or (D) GI, peritoneum or ammamry glands (MGs) of mice injected with shControl, sh413, sh413 WT, or sh413 C3A 4T1 cells. (E) Clinical score plot of mice implanted with shControl, sh413, sh413 WT, or sh413 C3A 4T1 cells. Clinical signs were scored as described in FIG. 4. (F) Kaplan Meier survival plot of mice implanted with with shControl, sh413, sh413 WT, or sh413 C3A 4T1 cells. Error bars represent standard deviations. Significance was taken at P<0.05* and P<0.01**. n.s=not significant.

FIG. 30. BST-2 dimerization mediates collective cell clustering and promotes tumorigenesis. (A) Representative images of crystal violet-stained colonies from a soft agar assay showing anchorage-independent growth of 4T1 cells expressing shControl (shCTL), sh413, or sh413 cells in which hBST-2 (sh413 WT) or C3A hBST-2 (sh413 C3A) was rescued. Clones were imaged at 10×. (B) Representative images of tumor cells tracked in vivo with IVIS imaging at different time points form mice injected with shControl, sh413, sh413 WT or sh413 C3A 4T1 cells. (C) Tumor volume (TV) computed as TV=0.5 (length*width2) over time in mice injected with shControl, sh413, sh413 WT or sh413 C3A 4T1 cells. (D) MCF-7 cells expressing Vector, WT, or C3A BST-2 were cultured onto plates that were previously coated with 95% Ethanol (Vehicle) or Poly-HEMA and incubated at 37° C. for 48 hours. Cells were collected to do viability assays using trypan blue staining. Significance was taken at P<0.05*. n.s=not significant.

FIG. 31. Treating tumor bearing mice with WT peptide results in slower tumor growth. BALB/c mice were injected with 300,000 4T1-luc cells; once tumors reached a volume of 100 mm3 treatment was started. Mice were given either a WT form of BST-2 (WT peptide) which encompasses most of its extracellular domain that contains all three cysteines or with a mutant peptide where all three cysteines were replaced with alanines (C3A peptide). Mice were injected intratumorally every three days with a concentration of 0.3 ug/ul at 0.33 ul/mm3 tumor. (A) Tumor volumes were measured daily with the formula TV=0.5(Length*Width2). In addition, (B) tumor volume as a percent was calculated by multiplying the tumor volume in a particular day by 100 and dividing it by the tumor volume on the day treatment began. TV (%)=(TV on day X*100)/TV on day 1 of treatment. Error bar corresponds to SEM. Significance was taken at P<0.05(*) and numbers correspond to p-values that did not reach statistical significance.

FIG. 32. BST-2 dimerization controls virus (MMTV)-induced cancer cell resistance to anoikis. 96-well plates were coated with 50 ul of 95% Ethanol (Vehicle) or 50 ul of 12 mg/ml Poly-HEMA in 95% Ethanol (Poly-HEMA) and allow to dry for 72 hours. Poly-HEMA prevents cells from attaching to the plastic. Following, 4T1 shControl, sh413, sh413+WT hBST2 and sh413+C3A hBST2 either infected with MMTV at an MOI=0.1 or naive were plated at 20,000 cells/well and incubated at 37 degrees Celsius for 48 hours. Cells were collected to do viability analyses using tryphan blue (panel A) and MTT assays (panel B). In addition, cells were collected and pelleted to isolate RNA and protein. Levels of BST-2 and the apoptotic factors Bim and Caspase-3 were analyzed by RT-qPCR normalizing with GAPDH (panels C and D) and Caspase-3 proteins levels were analyzed by western blot using GAPDH as a loading control (panel E). Error bar corresponds to standard deviations. Significance was taken at P<0.05(*) and P<0.01(**).

FIG. 33. MMTV promotes invasion potential of breast cancer cells in a BST-2 dependent manner. A) The apical chamber of 24-well cell culture inserts were seeded with previously starved shControl or sh413 expressing 4T1 cells (150,000) in serum-free medium. Culture medium containing 30% FBS was added to the basal chamber of the unit and cells were allowed to migrate through the membranous barrier for 20 hours at 37° C. Non-migrated cells were washed off, migrated cells were fixed with 4% PFA for 5 minutes, washed twice with 1×PBS, permeabilized with 100% methanol for 25 minutes, labeled with Giemsa stain (for 15 minutes at RT) and imaged using a Nikon Eclipse Ti microscope adjusted with a Nikon digital sight camera. A) Representative images of 4T1 cells expressing shControl or an shRNA targeting BST-2 (sh413) either uninfected (Naive) or infected with MMTV (Infected) at MOI=0.1 that invaded through matrigel. A similar experiment was performed were cells that did not invade (Top) and cells that invaded (Bottom) through matrigel were collected to analyze B) MMTV DNA levels and C) MMP-9 and D) MMP-2 RNA levels. RNA levels were analyzed by RT-qPCR normalizing with GAPDH. Error bar corresponds to standard deviations. Significance was taken at P<0.05(*).

FIG. 34. The Y×Y motif on the cytoplasmic tail of BST-2 regulates cancer cell migration and invasion. A) BST-2 Y×Y motif is not required in the interaction of cancer cells with fibronectin. 96-well plates were coated with fibronectin in 2% BSA at a concentration of 50 ug/ml. Then, MCF-7 cells expressing and empty pCDNA3.1 plasmid (Vector), a WT BST-2-containing plasmid (WT) and a mutant BST-2-expressing plasmid (Y6,8A) in which two tyrosines at positions 6 and 8 were replaced with alanines were labeled with green PKH67 dye and added to fibronectin or collagen coated wells at 20,000 cells/well. The plate was incubated at 37 degrees C. for 4 hours and then washed twice. Following, fluorescence was read with a TECAN plate reader at excitation 485, emission 535 nm wavelengths and representative wells imaged with a confocal microscope. B) BST-2 Y×Y motif is not required for anchorage-independent growth. 24-well plates were coated with 500 μl of 0.5% agar and allowed to solidify. Following, MCF-7 cells expressing Vector, WT or Y6,8A were plated at 1,250 cells/well in 500 μl of 0.35% agar. 250 μl of the appropriate growth medium was added on top of the agar layer. Growth medium was replaced twice a week and cells were allowed to form colonies for 25 days. Colonies were stained with crystal violet and imaged. C) Y×Y motif of BST-2 is involved in cancer cell migration. MCF-7 cells expressing Vector, WT or Y6,8A were plated to confluency in a 24-well plate, scratched and imaged at 24 hours after. Black doted lines represent the wound edge. D) Y×Y motif of BST-2 is involved in cancer cell invasion. The apical chamber of 24-well cell culture inserts were seeded with previously starved MCF-7 cells expressing Vector, WT or Y6,8A cells (150,000) in serum-free medium. Culture medium containing 30% FBS was added to the basal chamber of the unit and cells were allowed to migrate through the membranous barrier for 20 hours at 37° C. Non-migrated cells were washed off, migrated cells were fixed with 4% PFA for 5 minutes, washed twice with 1×PBS, permeabilized with 100% methanol for 25 minutes, labeled with Giemsa stain (for 15 minutes at RT) and imaged using a Nikon Eclipse Ti microscope adjusted with a Nikon digital sight camera. Representative images are shown.

FIG. 35. BST-2 structure and functions. BST-2 is a type II transmembrane protein with a N-terminal cytoplasmic tail (CT) followed by a transmembrane domain (TM), a coiled-coil domain an a glycosylphosphatidylinositol (GPI) anchor which is embedded in lipid rafts in the cell membrane. The amino acid sequence of human BST-2 is depicted in the gray box where the amino acids were color coded to their respective domain. Numbers on top of amino acids correspond to amino acid location. Underneath the amino acid sequences there are colored boxes that correspond to different functions and characteristics of BST-2 including NF-κB activation (yellow), dimerization (blue), virus tethering (light purple), endocytosis/adaptor protein (AP) binding (green), sites of glycosylation (dark red), Vpu binding (orange), actin association (purple), motif for ADCC inducion (brown) and also some hypothetical characteristics/functions including sites of ubiquitination (light orange), cell-to-cell adhesion (light green) and induction of cancer-promoting genes such as matrix metalloproteinases, CXCR4, CXCL12 and other signaling molecules (gray). BST-2 contains two translational start sites at methionine 1 and 13 (red) generating a long and short isoform, respectively. The short isoform cannot induce NF-κB activation since it lacks the Y×Y motif. BST-2 forms homo-dimers and -tetramers through 3 conserved cytosines at positions 53, 63 and 91 but leucines at positions 70 and 123 are also important since they maintain the structure of the protein. These amino acids are also important for its tethering function which also requires the C-terminal GPI anchor. Furthermore, BST-2 contains two KKXX motifs and a DXE motif that may be recognized by COP I and COP II respectively. COP vesicles are involved in intracellular trafficking between the ER and the Golgi. Lastly, BST-2 contains an ILV motif (on TM) and a YXXC (on CT) motif that may be recognized by the retromer which recycles membrane proteins from early endosomes back to the plasma membrane.

FIG. 36. In silico analyses of BST-levels and circulating tumor cell cluster formation. (A) BST-2 RNA levels (presented as RPKM units) from circulating tumor cells (CTCs) singlets and clusters isolated from blood of patients with metastatic breast cancer. (B) Intrapatient comparison of BST-2 levels from CTC singlets and clusters of 10 different patients. Samples with RPKM values of zero were excluded from the study. Data were from GEO dataset GSE51827 [15]. (B) BST-2 expression vs BIM expression from circulating tumor cell (CTC) singlets (green) and clusters (red). r² value depicts an strong inverse correlation between BST-2 and BIM in CTC clusters. P-value is also depicted. Data does not reach statistical significance due to lack of enough data points but data show a good trend. Patients with RPKM values of zero for either BST-2 or BIM were excluded form the study. Error bars correspond to SEM. Significance was taken at P<0.01**.

FIG. 37. BST-2 promotes survival of breast cancer cells by endowing cancer cells resistance to anoikis. (A and B) 4T1 cells expressing shControl, sh413, sh13 WT or sh413 C3A were cultured in plates that were previously coated with (A) 95% Ethanol (Vehicle) or (B) Poly-HEMA (prevents cell attachment) and incubated at 37° C. Cells were collected 48 hours later and used for (C) MTT assay for assessment of viability; (D and E) assessment of the levels of BST-2 and BIM (pro-apoptotic factor) mRNA; and (F) analysis of the level of Cleaved Caspase-3 (active form) and BIM at the protein level. Levels of mRNA were analyzed by RT-qPCR normalizing with GAPDH. Protein levels were analyzed by western blot using GAPDH as the loading control. Error bars corresponds to SEM for viability assay and to standard deviations for RNA data. Significance was taken at P<0.05* and P<0.01**. n.s=not significant.

FIG. 38. BST-2-dimerization mutant is not phosphorylated at its cytoplasmic tail. 4T1 shControl, sh413, sh413 WT and sh413 C3A cells were plated on 6-well plates and treated with PBS (Vehicle). To induce BST-2 dimerization and the subsequent phosphorylation of BST-2 at its cytoplasmic tail, cells were treated with 200 ng/well recombinant BST-2 (rBST-2) for 1 hour. (A) Representative western blot images of lysates from vehicle or rBST-2-treated cells blotted for phosphorylated tyrosines (pY) and GAPDH used as loading control. (B) Representative western blot images of vehicle or rBST-2-treated 4T1 cells following immunoprecipitation with anti-BST-2 antibodies and blotted for pY. (C) Cartoon describing the pathway of events.

FIG. 39. BST-2-dimerization incompetent cells are unable to respond to TPA as a survival signal. (A) 4T1 shControl, sh413, (B) sh413 WT and sh413 C3A cells were plated on 6-well plates and treated with DMSO (Vehicle), 20 nM of TPA (survival signal), 1 uM of MG132 (proteosome inhibitor) or TPA+MG132 (TPA/MG132) for 24 hours. (A & B) shows representative western blot images for BIM, phosphorylated ERK1/2 (pERK1/2), and GAPDH, used for loading control. (C) Cartoon describing the pathway of events.

FIG. 40. ERK is involved in BST-2-mediated BIM phosphorylation. (A) 4T1 sh413 WT and sh413 C3A cells were plated on 6-well plates and treated with DMSO (Vehicle), 20 nM of TPA (survival signal), 20 uM of FR180204 (ERK1/2 inhibitor) or TPA+FR180204 (TPA/FR180204) for 24 hours. Representative western blot images blotting for phosphorylated BIM S69 (pBIM), phosphorylated ERK1/2 (pERK1/2), total ERK1/2 (ERK1/2), phosphorylated JNK T183/Y185 (pJNK), and GAPDH. Numbers underneath blots correspond to band quantifications normalized using GAPDH (relative units). (B) Cartoon describing the pathway of events.

FIG. 41. Creation of MDA-MB-231 cells stably expressing various levels of BST-2. MDA-MB-231 cells were transfected with a scramble shRNA control and 4 different BST-2-targeting shRNAs. After selection for cells stably expressing these shRNAs, cells were collected and used for analysis of BST-2 levels by FACS.

FIG. 42. Effects of BST-2 silencing on migration of Triple Negative Breast Cancer (TNBC) cells. A & C) Representative images of cell migration performed using A) Boyden chambers and C) scratch assay. B) Quantification of migrated cells using 20× images (n=3). Suppression of BST-2 expression significantly reduced rate of cancer cell migration through the A) membranous chamber and into the B) scratch wound. Representative images taken at 4×, 10× and 20×. Dotted red lines on C represent the wound edge and numbers within the wounds represent the relative wound length. Data show that reducing BST-2 levels using shRNAs impair the ability of TNBC cells to migrate in 2D and 3D platforms. Images acquired at 20× were used for quantification.

FIG. 43. BST-2 promotes invasion of MDA-MB-231 TNBC cells In Vitro Effects of BST-2 silencing on invasion of TNBC cells. A) MDA-MB-231 cells expressing shControl or shBST-2 were plated in low-serum medium on Matrigel-coated cell inserts and allowed to migrate for 24 h. Cells were stained with Giemsa stain. Representative images taken at 4× and 10× are shown. B) Quantification of invaded cells using 10× images (n=3). Data show that reducing BST-2 level impairs the ability of TNBC cells to invade through a ECM-like matrix. Images acquired at 20× were used for quantification.

FIG. 44. Suppression of BST-2 in cancer cells decreases lung metastasis in vivo. (A) Representative images of luciferase activity in mice injected via tail vein with shCTL, sh413 or sh413 WT 4T1 cells over 42 days. (B) Representative images of lungs from mice shown in A. Arrows indicate lung nodules. (C) Quantification of total lung tumors found post mortem in each mouse. (D) Average spleen weight of mice shown in A. (E) Percent survival of shCTL, sh413 or sh413 WT 4T1 injected mice post injection. Significance was taken at P<0.05 (*). Error bars correspond to standard deviations.

FIG. 45. Suppression of BST-2 in cancer cells elevates levels of pro-apoptotic factors in the lungs of tumor-bearing mice. (A) BIM and Caspase-3 mRNA data of lung tissues from mice injected with high-BST-2 expressing shCTL 4T1 cells or BST-2-suppresed sh413 4T1 cells. (B) Representative western blots showing BIM and cleaved Caspase-3 (cCaspase-3) levels in lungs from tail vein injected BALB/c mice. (C & D) Quantitation of protein levels of (C) BIM and (D) cCaspase-3 from lungs of shCTL, sh413 or sh413 WT injected mice (n=3). GAPDH was used for RNA and protein normalization. Significance was taken at P<0.05 (*). Error bars correspond to standard deviations. RU=relative units.

FIG. 46. A truncated form of B49 (B49nc) inhibits cancer cell adhesion similar to B49. Quantification of PKH67Green-labeled shControl-expressing MDA-MB-231 cells that were allowed to adhere to MDA-MB-231 shControl monolayers previously blocked with PBS (Vehicle), recombinant BST-2 (rBST-2), B49 peptide or a truncated form of B49 that lacks amino acids from both N- and C-terminus (B49nc). Moreover, MDA-mb-231 SHcONTROL CELLS WERE ADDED TO mda-mb-231 ShBST-2 monolayers (on shBST-2). (Each data point corresponds to six different wells analyzed. Error bars correspond to SEM. Significance was taken at P<0.0005*** or P<0.0001****.

FIG. 47. Advantage of targeting BST-2 over other current drug targets. BST-2 is highly expressed in about 68% of all breast cancers. This percent is much higher than any of the expression of other breast cancer therapeutic targets such as estrogen receptor (ER, 58%), progesterone receptor (PR, 11.1%), HER2 (36%), or Myc (0%). Moreover, analysis of BST-2 expression in breast cancer tissues showed that BST-2 is highly expressed in 72% of all breast cancers. Thus, on average, BST-2 is highly expressed in about 70% of all breast cancers based on data from proteinatlas.org and from the TCGA.

### DETAILED DESCRIPTION

Disclosed are compositions, kits, and methods for treating and/or diagnosing cancer in a subject in need thereof, in particular in a subject having a cancer associated with BST-2 expression or BST-2 biological activity. The compositions, kits, and methods may be further described as follows.

Unless otherwise specified or indicated by context, the terms “a”, “an”, and “the” mean “one or more.” In addition, singular nouns such as “BST-2 inhibitor” should be interpreted to mean “one or BST-2 inhibitors.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The terms “subject,” “patient,” or “host” may be used interchangeably herein and may refer to human or non-human animals. Non-human animals may include, but are not limited to non-human primates, dogs, cats, and mice.

The terms “subject,” “patient,” or “individual” may be used to a human or non-human animal having or at risk for acquiring a cell proliferative disease or disorder. Individuals who are treated with the compositions disclosed herein may be at risk for cancer or may have already acquired cancer including cancers such as breast cancer.

The presently disclosed compositions, kits, and methods may be utilized to treat cancers or hyperproliferative disorders that are associated with BST-2 expression or BST-2 biological activity, which may include, but are not limited to adenocarcinoma, lymphoma, melanoma, myeloma, sarcoma, and teratocarcinoma and particularly cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus.

Bone marrow stromal antigen 2 (BST-2) is a 180 amino acid transmembrane protein having the amino acid sequence (SEQ ID NO:1):

| | |
|---|---|
| 1 | mastsydycr vpmedgdkrc klllgigilv lliivilgvp liiftikans eacrdglrav |
| 61 | mecrnvthll qqelteaqkg fqdveaqaat cnhtvmalma sldaekaqgq kkveelegei |
| 121 | ttlnhklqda saeverlrre nqvlsvriad kkyypssqds ssaaapqlli vllglsallq |

The peptides contemplated herein may comprise a contiguous amino acid sequence of SEQ ID NO:1 of at least about 5, 10, 20, 30, 40, 50, or more amino acids and preferably the peptides contemplated herein inhibit dimerization of BST-2 (e.g., by binding to full-length BST-2 and acting as a decoy that prevents dimerization of the bound full-length BST-2 with another full-length BST-2 molecule). The peptides contemplated herein may comprise at least a portion of the ectodomain of BST-2, for example, a contiguous amino acid sequence from amino acids 47-95 (SEQ ID NO:2):

| | |
|---|---|
| 47 | ans eacrdglrav |
| 61 | mecrnvthll qqelteaqkq fqdveaqaat cnhtv |

The compositions disclosed herein may be formulated as pharmaceutical composition for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants as known in the art. Further, the compositions may include preservatives. The compositions also may include buffering agents.

The pharmaceutical compositions may be administered therapeutically. In therapeutic applications, the pharmaceutical compositions are administered to a patient in an amount sufficient to elicit a therapeutic effect (e.g., a therapeutic effect in response to cancer which irradicates or at least partially arrests or slows growth of the cancer or inhibits metastasis of the cancer (i.e., a “therapeutically effective dose”)).

Reference is made herein to polypeptides and pharmaceutical compositions comprising polypeptides such as BST-2 and variants of BST-2 such as BST-2 peptides. An exemplary polypeptide may comprise the amino acid sequence of any of SEQ ID NOs:1, 2, or 14, or may comprises an amino acid sequence having at least about 80%, 90%, 95%, 96%, 97%, 98%, or 99% sequence identity to any of SEQ ID NOs:1, 2, or 14. Variant polypeptides may include polypeptides having one or more amino acid substitutions, deletions, additions and/or amino acid insertions relative to a reference polypeptide. Also disclosed are nucleic acid molecules that encode the disclosed polypeptide (e.g., polynucleotides that encode the polypeptide of any of SEQ ID NOs:1, 2, or 14 or variants thereof). The disclosed BST-2 polypeptides, BST-2 peptides or variants thereof may exhibit one or more biological activities associated with BST-2, which may include, but are not limited to inhibiting homodimerization of BST-2 (e.g., as a decoy that binds to BST-2 and prevents BST-2 from homodimerizing).

The terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

The polypeptides and peptides contemplated herein may include conservative amino acid substitutions relative to a reference peptide or polypeptide. For example, a variant BST-2 polypeptide or peptide may include conservative or non-conservative amino acid substitutions relative to the natural BST-2 polypeptide or peptide. “Conservative amino acid substitutions” are those substitutions that are predicted to interfere least with the properties of the reference polypeptide. In other words, conservative amino acid substitutions substantially conserve the structure and the function of the reference protein. The following table provides a list of exemplary conservative amino acid substitutions.

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A variant BST-2 polypeptide or peptide may include a deletion relative to a reference BST-2 polypeptide or peptide. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation of a reference polypeptide or a 5′-terminal or 3′-terminal truncation of a reference polynucleotide).

A variant BST-2 polypeptide or peptide may comprise a fragment of a reference BST-2 polypeptide or peptide A “fragment” is a portion of an amino acid sequence or a polynucleotide which is identical in sequence to but shorter in length than a reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 25, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule. The term “at least a fragment” encompasses the full length polynucleotide or full length polypeptide.

A variant may include an insertion or addition relative to a reference polypeptide sequence. The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200 or more amino acid residues.

Variants of BST-2 may include non-naturally occurring polypeptides or peptides comprising a contiguous amino acid sequence of BST-2 but lacking an N-terminal methionine as in present in naturally occurring BST-2. Variants of BST-2 may include non-naturally occurring polypeptides or peptides comprising a contiguous amino acid sequence of BST-2 except that the contiguous amino acid sequence includes a amino acid substation in which a cysteine is replaced by another amino acid such as alanine. Variants of BST-2 may include non-naturally occurring polypeptides or peptides that act as dominant negative inhibitors of BST-2 homodimerization.

Fusion proteins also are contemplated herein. A “fusion protein” refers to a protein formed by the fusion of at least one molecule of BST-2 (or a fragment or variant thereof) to at least one molecule of a heterologous protein (or fragment or variant thereof), which may include a protein that facilitates transport of the fusion protein across the cell membrane (e.g., penetratin, TAT, low molecular weight protamine, and poly(arginine)₈). A BST-2 fusion protein comprises at least a fragment or variant of the heterologous protein and at least a fragment or variant of BST-2, which are associated with one another, preferably by genetic fusion (i.e., the BST-2 fusion protein is generated by translation of a nucleic acid in which a polynucleotide encoding all or a portion of the heterologous protein is joined in-frame with a polynucleotide encoding all or a portion of BST-2 or a fragment or variant thereof). The heterologous protein and BST-2 protein, once part of the BST-2 fusion protein, may each be referred to herein as a “portion”, “region” or “moiety” of the BST-2 fusion protein (e.g., a “a heterologous protein portion” or a “BST-2 protein portion”).

Conjugate proteins also are contemplated herein. A “BST-2 conjugate protein” refers to a protein formed by the conjugation (i.e., covalently bonding) of at least one molecule of BST-2 (or a fragment or variant thereof) to at least one molecule of a heterologous protein (or fragment or variant thereof), which may include a protein that facilitates transport of the fusion protein across the cell membrane (e.g., penetratin, TAT, low molecular weight protamine, and poly(arginine)₈). A BST-2 conjugate protein comprises at least a fragment or variant of the heterologous protein and at least a fragment or variant of BST-2, which are associated with one another by covalent bonding. The heterologous protein and BST-2 protein, once part of the BST-2 conjugate protein, may each be referred to herein as a “portion,” “region” or “moiety” of the BST-2 conjugate protein (e.g., “a heterologous protein portion” or a “BST-2 protein portion”).

Suitable heterologous proteins for the contemplated BST-2 fusion protein and BST-2 conjugate proteins may include a protein or peptide that facilitates transport of the fusion protein or conjuage protein across the cell membrane. Suitable proteins that facilitate transport of the fusion protein or conjuage protein across the cell membrane may include, but are not limited to penetratin, TAT, low molecular weight protamine, and poly(arginine)₈.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” of a particular polypeptide sequence may be defined as a polypeptide sequence having at least 50% sequence identity to a reference polypeptide sequence over a certain length of the reference poplypeptide sequence using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of the reference polypeptide sequence. A “variant” may have one or more functional activities of the reference polypeptide sequence.

The disclosed polypeptides may be modified so as to comprise an amino acid sequence or modified amino acids, such that the disclosed polypeptides cannot be said to be naturally occurring. In some embodiments, the disclosed polypeptides are modified and the modification is selected from the group consisting of acylation, acetylation, formylation, lipolylation, myristoylation, palmitoylation, alkylation, isoprenylation, prenylation, and amidation. An amino acid in the disclosed polypeptides may be thusly modified, but in particular, the modifications may be present at the N-terminus and/or C-terminus of the polypeptides (e.g., N-terminal acylation or acetylation, and/or C-terminal amidation). The modifications may enhance the stability of the polypeptides and/or make the polypeptides resistant to proteolysis and provide a longer in vivo half-life for the polypeptides comprising the modifications.

“Substantially isolated or purified” amino acid sequences are contemplated herein. The term “substantially isolated or purified” refers to amino acid sequences that are removed from their natural environment, and are at least 60% free, preferably at least 75% free, and more preferably at least 90% free, even more preferably at least 95% free from other components with which they are naturally associated.

A “composition comprising a given polypeptide” and a “composition comprising a given polynucleotide” refer broadly to any composition containing the given polynucleotide or amino acid sequence. The composition may comprise a dry formulation or an aqueous solution. The compositions may be stored in any suitable form including, but not limited to, freeze-dried form and may be associated with a stabilizing agent such as a carbohydrate. The compositions may be aqueous solution containing salts (e.g., NaCl), detergents (e.g., sodium dodecyl sulfate; SDS), and other components (e.g., Denhardt's solution, dry milk, salmon sperm DNA, and the like).

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed below).

Percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The disclosed pharmaceutical composition may comprise the disclosed BST-2 polypeptides, BST-2 peptides, or variants thereof at any suitable dose. Suitable doses may include, but are not limited to, about 0.01 μg/dose, about 0.05 μg/dose, about 0.1 μg/dose, about 0.5 μg/dose, about 1 μg/dose, about 2 μg/dose, about 3 μg/dose, about 4 μg/dose, about 5 μg/dose, about 10 μg/dose, about 15 μg/dose, about 20 μg/dose, about 25 μg/dose, about 30 μg/dose, about 35 μg/dose, about 40 μg/dose, about 45 μg/dose, about 50 μg/dose, about 100 μg/dose, about 200 μg/dose, about 500 μg/dose, or about 1000 μg/dose.

The disclosed BST-2 polypeptides, BST-2 peptides, or variants thereof may be administered at any suitable dose level. In some embodiments, a subject in need thereof is administered a BST-2 polypeptide, a BST-2 peptide, or variant thereof at a dose level of from about 1 ng/kg up to about 2000 ng/kg. In some embodiments, a BST-2 polypeptide, a BST-2 peptide, or variant thereof is administered to the subject in need thereof at a dose level of at least about 1 ng/kg, 2 ng/kg, 5 ng/kg, 10 ng/kg, 20 ng/kg, 50 ng/kg, 100 ng/kg, 200 ng/kg, 500 ng/kg, 1000 ng/kg or 2000 ng/kg. In other embodiments, a BST-2 polypeptide, a BST-2 peptide, or variant thereof is administered to the subject in need thereof at a dose level of less than about 2000 ng/kg, 1000 ng/kg, 500 ng/kg, 200 ng/kg, 100 ng/kg, 50 ng/kg, 20 ng/kg, 10 ng/kg, 5 ng/kg, 2 ng/kg, or 1 ng/kg. In further embodiments, a BST-2 polypeptide, a BST-2 peptide, or variant thereof is administered to a subject in need thereof within a dose level range bounded by any 1 ng/kg, 2 ng/kg, 5 ng/kg, 10 ng/kg, 20 ng/kg, 50 ng/kg, 100 ng/kg, 200 ng/kg, 500 ng/kg, 1000 ng/kg or 2000 ng/kg.

The disclosed the BST-2 polypeptides, BST-2 peptides, or variants thereof may be administered under any suitable dosing regimen. Suitable dosing regimens may include, but are not limited to, daily regimens (e.g., 1 dose/day for 1, 2, 3, 4, 5, 6, 7 or more days), twice daily regimens (e.g., 2 doses/day for 1, 2, 3, 4, 5, 6, 7 or more days), and thrice daily regiments (e.g., 3 doses/day for 1, 2, 3, 4, 5, 6, 7 or more days). Suitable regiments also may include dosing every other day, 3 times/week, once a week, for 1, 2, 3, 4, or more weeks.

The disclosed BST-2 polypeptides, a BST-2 peptides, or variants thereof (or pharmaceutical compositions comprising the disclosed BST-2 polypeptides, a BST-2 peptides, or variants thereof) may be administered to a subject in need thereof by any suitable route. In some embodiments, the disclosed BST-2 polypeptides, a BST-2 peptides, or variants thereof are administered to a subject in need thereof via an injectable delivery route selected from the group consisting of intradermal, intramuscular, intraperitoneal, intravenous, subcutaneous, intratumorally, or epidural routes. In another embodiment, the disclosed BST-2 polypeptides, a BST-2 peptides, or variants thereof are administered to a subject near a site of a tumor or cancer.

The disclosed methods may include administering to a subject in need thereof a therapeutic agent that inhibits the expression of BST-2 as known in the art. In some embodiments, the therapeutic agent is an RNA-interference based therapeutic. (See, e.g., Bobbin et al., “RNA Interference (RNAi)-Based Therapeutics: Delivering on the Promise?”, Ann. Rev. of Pharma. And Toxic., Vol. 56: 103-22, January 2016, the content of which is incorporate herein by reference in its entirety). RNAi-based therapeutics may include but are not limited to small hairpin RNAs (shRNAs), small interfering RNAs (siRNAs), microRNAs (miRNAs), and/or PIWI-interacting RNAs (piRNAs) and/or vectors that express shRNAs, siRNAs, miRNAs, and/or piRNAs, where the RNAi-based therapeutics inhibit expression of BST-2.

The disclosed methods may include determining or detecting the methylation status of the BST-2 gene and/or the promoter for the BST-2 gene in genomic DNA isolated from breast cancer cells. For example, determining or detecting the methylation status of the BST-gene may include determining or detecting hypomethylation of the promoter of the BST-2 gene. Methylation status may be analyzed as understood in the art. (See, e.g., Kurdyukov et al., “DNA Methylation Analysis: Choosing the Right Method,” Review, Biology 2016, 5, 3; doi:10.3390/biology5010003, the content of which is incorporated herein by reference in its entirety). In some embodiments, the disclosed methods include treating genomic DNA isolated from breast cancer cells with a bisulfite reagent that converts non-methylated cytosines to uracil. As such, contemplated herein is BST-2 DNA isolated from breast cancer cells that has been treated with a bisulfite reagent to convert non-methylated cytosines to uracil in order to prepare a non-naturally occurring form of BST-2 DNA.

### EXAMPLES

The following examples are illustrative and are not intended to limit the disclosed and claimed subject matter.

### Example 1

Reference is made to “Bone marrow stromal antigen 2 expressed in cancer cells promotes mammary tumor growth and metastasis,” Wadie D. Mahauad-Fernandez, Kris A. DeMali, Alicia K. Olivier, and Chioma M. Okeoma, Breast Cancer Research (2014) 16:493, DOI 10.1186/s13058-014-0493-8, published on Dec. 13, 2014, the content of which is incorporated herein by reference in its entirety.

Title: Bone Marrow Stromal Antigen 2 Expressed in Cancer Cells Promotes Mammary Tumor Growth and Metastasis

Abstract

### Introduction

Several innate immunity genes are overexpressed in human cancers and their roles remain controversial. Bone marrow stromal antigen 2 (BST-2) is one such gene whose role in cancer is not clear. BST-2 is a unique innate immunity gene with both antiviral and pro-tumor functions and therefore can serve as a paradigm for understanding the roles of other innate immunity genes in cancers.

Methods:

Meta-analysis of tumors from breast cancer patients obtained from the Gene Expression Omnibus (GEO) and The Cancer Genome Atlas (TCGA) datasets were evaluated for levels of BST-2 expression and for tumor aggressiveness. In vivo, we examined the effect of knockdown of BST-2 in two different murine carcinoma cells on tumor growth, metastasis, and survival. In vitro, we assessed the effect of carcinoma cell BST-2 knockdown and/or overexpression on adhesion, anchorage-independent growth, migration, and invasion.

Results:

BST-2 in breast tumors and mammary cancer cells is a strong predictor of tumor size, tumor aggressiveness, and host survival. In humans, BST-2 mRNA is elevated in metastatic and invasive breast tumors. In mice, orthotopic implantation of mammary tumor cells lacking BST-2 increased tumor latency, decreased primary tumor growth, reduced metastases to distal organs, and prolonged host survival. Furthermore, we found that the cellular basis for the role of BST-2 in promoting tumorigenesis include BST-2-directed enhancement in cancer cell adhesion, anchorage-independency, migration, and invasion.

### Conclusions

BST-2 contributes to the emergence of neoplasia and malignant progression of breast cancer. Thus, BST-2 may (1) serve as a biomarker for aggressive breast cancers, and (2) be a novel target for breast cancer therapeutics.

### Introduction

The oncogenesis of breast cancer involves multiple events, including genetic and epigenetic alterations in the behavior of normal and malignant cells, as well as other cells that interact with cancer cells [1]. Such alterations modulate the functions of key host genes, which in turn affect cancer cell behavior including self-sufficiency in growth signals, adhesion, invasion, motility, and survival. Our understanding of specific genes linked to the development and progression of mammary cancer is unraveling. These genes have enabled the development of targeted therapeutics against mammary cancers that are dependent on such genes. However, the goal of eliminating breast cancer has not been met partially because not all cancer driver genes have been identified. In particular, it is not clear how overexpression of innate immunity genes in cancer cells endow these cells tumorigenic potential.

Innate immunity is crucial to host defense. However, some innate immunity genes play paradoxical roles as they prevent [2] and/or promote [3] cancer through mechanisms that are not well defined. It has been shown that the innate immunity gene called bone marrow stromal antigen 2 (BST-2), also known as tetherin, CD317, and HM1.24 is overexpressed in several cancers [4-11]. BST-2 is an interferon-inducible type II transmembrane protein that functions as a potent nuclear factor kappa binding (NF-κB) activator [12]. BST-2-mediated NF-κB activation occurs through the YXY motif on the cytoplasmic domain of BST-2 and interaction with TAK1 is required [13,14]. The activation of NF-κB by BST-2 results in increased production of immune-inflammatory mediators that may inhibit viral replication [13], but may also promote tumorigenesis. In addition to the NF-κB-regulating role, BST-2 is reputed for its tethering and antiviral functions, as its overexpression tethers/retains nascent virions on the surface of infected cells and prevents infection of new target cells [15-17]. The tetherin function of BST-2 has been shown to be involved in cell to cell interactions because BST-2 mediates the adhesion of monocytes to endothelial cells [18]; a function that could promote intravasation of immune cells.

Although overexpression of BST-2 tethers virions on the cell membrane and negatively regulates virus replication, it is likely that elevated BST-2 expression might positively influence cancer cell behavior [6, 7, 9, 10, 19]. It has been suggested that increased cancer cell adhesion and resistance to apoptosis in vitro is linked to BST-2 expression [18, 20, 21]. However, the functional consequence of BST-2 expression in tumor tissues and cells is completely unknown and there has been no direct demonstration of the involvement of BST-2 in breast tumorigenesis.

Given the role of BST-2 in innate immunity—including its role in NF-κB activation and subsequent transcription of NF-κB-dependent genes, as well as the presence of high levels of BST-2 in breast tumors [21], we hypothesized that BST-2 may promote mammary tumorigenesis. Here, we studied the clinical consequences of BST-2 expression in breast tumors, the functional role of BST-2 in mammary tumorigenesis, and the cellular basis for BST-2-mediated effect on mammary tumorigenesis.

Methods

Cell Lines.

E0771 (a medullary breast adenocarcinoma cell line from C57BL/6 mouse strain) was purchased from CH3 Bio-Systems (Amherst, N.Y., USA). 4T1 (a mouse mammary carcinoma cell line from BALB/c mouse strain) was provided by Dr. Lyse Norian of the University of Iowa. HMLE (Normal human mammary epithelial cell line), MCF-7 cells (luminal A human breast cancer cell line) and MDA-MB-231 cells (triple-negative human breast cancer cell line) were kindly provided by Dr. Weizhou Zhang of the University of Iowa.

Animals.

Five-week-old C57BL/6NCr and BALB/cAnNCr female mice were used. Mice were sacrificed when they became moribund. Tumor volume (TV) was calculated as: TV=0.5(length*width2) [22]. Tumor latency was calculated as the number of tumor-free injected mice/number of injected mice×100. To assess morbidity, the following clinical score ranking was used: (0) no abnormal clinical signs, (1) ruffled fur but lively, (2) ruffled fur, activity level slowing, sick, (3) ruffled fur, eyes squeezed shut, hunched, hardly moving, very sick, (4) moribund and (5) dead [23]. Experiments involving mice were approved by the University of Iowa Animal Care and Use Committee (IACUC).

Mice Injections and Live Animal Imaging.

Orthotopic mammary tumors were generated by implanting 1.5×105 cancer cells in 200 μl of phosphate-buffered saline (PBS) into the mammary fat pad of five-week-old female mice. Prior to imaging, mice were anesthetized, weighed and injected intraperitoneally with D-luciferin. Mice were imaged using the Xenogen IVIS three dimensional optical imaging system (Caliper Life Sciences, Hopkinton, Mass., USA). Luciferase was quantified with Living Image Software (Caliper Life Sciences).

Histology.

Gastrointestinal samples were rolled for processing to allow visualization of mesenteric tumors. Fixed tissues were paraffin embedded, sectioned at 4 μm, and stained with hematoxylin and eosin (H&E). Spleen and lung sections were imaged using a BX51 Olympus microscope (Olympus, Tokyo, Japan). Gastrointestinal slides were scanned with an Aperio ScanScope CS (Aperio Technologies, San Diego, Calif., USA).

Lentiviral Transduction.

E0771 and 4T1 cells were stably transduced with a construct expressing LV-CMV-firefly luciferase or an empty vector construct using lipofectamine following the manufacturer's instructions (Life Technologies, Carlsbad, Calif., USA). Stable transfectants were then transduced with lentiviral particles carrying BST-2-targeting sh137: CCGGC GCGATCTTGGTGGTCCTGTTCTCGAGAACAGGACCACCAAGATCGCGTTTTTG (SEQ ID NO:3); sh413: CCGGGCTTGAGAATGAAGTCACGAACTCGAGTTCGTGACTTCA TTCTCAAGCTTTTTG (SEQ ID NO:4); or a non-targeting shControl: CCGGCAACAAGATGAAGAGCACCAACTCGAGTTGGTGCTCTTCATCTTGTTGTTTTT (SEQ ID NO:5) using a previously described protocol [17]. Stable cells were generated by selection with the appropriate drug. The short hairpin RNA (shRNA) constructs were purchased from Sigma-Aldrich (St Louis, Mo., USA) (SHCLND-NM_198095) and lentiviral particles were generated at the Gene Transfer Vector Core at the University of Iowa.

Flow Cytometry.

Cell monolayers were washed with PBS and treated with Versene (Life Technologies). Single cells were stained with fluorescein isothiocyanate (FITC)-conjugated anti-mouse BST-2 (eBioscience, San Diego, Calif., USA), allophycocyanin (APC)-conjugated anti-human BST-2 (BioLegend, San Diego, Calif., USA), and appropriate immunoglobulin Gs (IgGs) [16,17] at 4° C. for 1 hour. After washing, cells were incubated with a fluorescent intercalator—7-aminoactinomycin D (7-AAD) (BioLegend) at 4° C. for 30 minutes to assess cell viability. Stained cells were quantified using a FACSCalibur flow cytometer (BD Biosciences, San Jose, Calif., USA). At least 10,000 events were collected per sample. Fluorescence-activated cell sorting (FACS) data were analyzed by Flowjo software (TreeStar, Ashland, Oreg., USA).

Reverse Transcriptase Quantitative Real-Time PCR (RT-qPCR).

Isolation of RNA was accomplished using the RNeasy mini kit (Qiagen, Venlo, Netherlands) according to the manufacturer's instructions. Equivalent amounts of DNase I (Qiagen)-treated RNA were reverse-transcribed with a high-capacity cDNA reverse transcription kit (Applied Biosystems, Carlsbad, Calif., USA). cDNA was amplified with target-specific primers (GAPDH-Forward: 5′-CCCCTTCATTGACCTCAACTACA-3′ (SEQ ID NO:6), Reverse: 5′-CGCTCCTGGAGGATGGTGAT-3′ (SEQ ID NO:7); mouse BST-2-Forward: TCAGGAGTCCCTGGAGAAGA (SEQ ID NO:8), Reverse: ATGGAGCTGCCAGAGTTCAC (SEQ ID NO:9); human BST-2 RT2 qPCR Primer Assays (SABiosciences, Frederick, Md., USA). RT-qPCR was carried out with an ABI 7500 FAST thermal cycler (Applied Biosystems) as previously described [24].

Western Blot.

Western blots were performed as previously described [24]. Blots were probed with anti-BST-2 (Abcam, Cambridge, UK) and anti-GAPDH (Santa Cruz Biotechnology, Dallas, Tex., USA) primary antibodies and appropriate IRDye secondary antibodies were used. Band detection and quantification were carried out with the Odyssey Infrared Imaging System (LI-COR Biosciences, Lincoln, Nebr., USA).

MEF Adhesion Assay.

Murine embryonic fibroblasts (MEFs) were grown to confluency in 6-well plates. Equivalent numbers (150,000) of cancer cells labeled with PKH67Green fluorescent cell linker, following the manufacturer's instructions (Sigma-Aldrich), were added to the MEF monolayer and allowed to incubate for 8 hours. Non-adhered cells were washed off and adhered cells imaged. Image J (NIH, Bethesda, Md., USA) was used to quantify the number of PKH67Greenpositive cells. In parallel, luciferase-expressing cancer cells were added to MEFs monolayers in 6-well plates for 8 hours. Non-adhered cells were washed off and adhered cells were treated with D-luciferin and imaged using IVIS. For luciferase assay, cancer cells plated in 96-well plate were used for quantitation of luciferase bioluminescence.

Collagen and Fibronectin Adhesion Assay.

Ninety-six-well plates were coated with 50 μg/ml of collagen or fibronectin. Plates were incubated at 37° C. for 2 hours. Nonspecific sites were blocked with 40 μl of 2 mg/ml bovine serum albumin (BSA) in PBS. Wells were washed once with PBS. Cancer cells were labeled with PKH67 Green fluorescent cell linker, following the manufacturer's instructions (Sigma-Aldrich). Labelled cells were added to pre-coated wells (20,000 cells/well) and allowed to adhere for 4 hours. Non-adhered cells were washed off with PBS and plates were read at 485 nm/535 nm (excitation/emission) wavelengths using a Tecan Infinite M200 Pro plate reader (Tecan, Maennedorf, Switzerland). Values are represented as relative fluorescence unit.

Scratch Assay.

Confluent monolayers of cancer cells plated in 12-well plates were scratched using a pipette tip. Fresh medium was added to the wells. Cells were allowed to migrate for 0, 6 or 24 hours before fixation (4% paraformaldehyde (PFA) for 45 minutes). Fixed cells were washed (1×PBS) and imaged with a Nikon Eclipse Ti microscope adjusted with a Nikon digital sight camera (Nikon, Tokyo, Japan). Images were processed and migrated cells counted using Image J software.

Boyden Chamber Assay.

The apical chamber of 24-well cell culture inserts (Merck Millipore, Billerica, Mass., USA) were seeded with previously starved sh137, sh413 or shControl transduced E0771 cells (150,000) in serum-free medium. Culture medium containing 30% FBS was added to the basal chamber of the unit and cells were allowed to migrate through the membranous barrier for 20 hours at 37° C. Non-migrated cells were washed off, migrated cells were fixed with 4% PFA for 5 minutes, washed twice with 1×PBS, permeabilized with 100% methanol for 25 minutes, labeled with Giemsa stain (for 15 minutes at room temperature) and imaged using a Nikon Eclipse Ti microscope adjusted with an X-cite series 120 LED fluorescence microscope light source and a Nikon digital sight camera. Images were processed using Image J software. Cells from five different fields were counted and averaged.

Cell Invasion Assay.

The apical chamber of 24-well cell culture inserts (Merck Millipore) were coated with 3 mg/ml of Matrigel (100 μl) (Sigma-Aldrich) and allowed to solidify for 5 hours. A total of 100,000 sh413- or shControl-transduced E0771 or 4T1 cells in serum-free medium were plated on top of the Matrigel layer. Culture medium containing 10% FBS and 5 μg/ml fibronectin (adhesive substrate) (Sigma-Aldrich) was added to the basal chamber of the unit (600 μl) and cells were allowed to invade through the membranous barrier for 24 hours at 37° C. Noninvasive cells were washed off; invasive cells were fixed with 4% PFA, permeabilized with 100% methanol, labeled with Giemsa stain and imaged as described in the previous paragraph. Images were processed using Image J software. Cells from five different fields were counted and averaged.

MTT Assay.

A total of 5,000 cells stably expressing sh137, sh413, or shControl were plated in 96-well plates. Cells were then incubated with 5 mg/ml MTT reagent for 3.5 hours followed by addition of MTT solvent (0.1% NP-40 and 4 mM HCl in isopropanol) and rocking for 15 minutes. Absorbance at 590 nm was read using a Tecan Infinite M200 Pro plate reader.

BrdU Assay.

A total of 5,000 cells were plated in 96-well plates for 24 hours. Bromodeoxyuridine or 5-bromo-2′-deoxyuridine (BrdU) (Calbiochem, Billerica, Mass., USA) assay was carried out according to the manufacturer's instructions. Absorbance at 450 nm was read using a Tecan Infinite plate reader. In parallel, 150,000 cells were plated in 24-well plates for 24 hours. Cells were incubated with BrdU label (1:2000) for 20 hours, treated with a fixative/denaturing solution (30 minutes) and incubated with an anti-BrdU antibody (1:1000) and rat anti-mouse BST-2 antibody (1:200, eBioscience) for 1 hour at room temperature. Cells were washed and incubated with Alexa Fluor 594 anti-rat (Invitrogen, Waltham, Mass., USA) and Alexa Fluor 488 anti-mouse (Invitrogen) secondary antibodies for 30 minutes at room temperature. Cells were stained with 4′,6-diamidino-2-phenylindole (DAPI)-containing Vectashield (Vector Laboratories, Burlingame, Calif., USA) and imaged using a Zeiss 710 confocal microscope (Carl Zeiss, Oberkochen, Germany). Images were processed using Image J software. BrdU label, fixative/denaturing solution, and anti-BrdU antibody were from BrdU (Calbiochem) assay.

Transformation Assay.

Agar was mixed in Roswell Park Memorial Institute medium (RPMI) with 20% FBS. A total of 500 μl of 0.5% agar was added to 24-well plate and allowed to solidify. Cells were plated at 1,250 cells/well in 500 μl of 0.35% agarose. Some 250 μl of the appropriate growth medium was added on top of the agarose layer. Growth medium was replaced twice a week. Colonies were stained with crystal violet and imaged. Colonies from five different fields were counted and averaged.

Meta-Analysis.

Three publically available Gene Expression Omnibus (GEO) datasets GSE4922 [25], GSE21422 [26] and GSE10797 [27] were used to analyze BST-2 expression with respect to tumor size, breast cancer classification and tumor type, respectively. From the GSE4922 dataset, only data from the Affymetrix Human Genome U133A Array were used (Affymetrix, Santa Clara, Calif., USA). From these data, only patients from Uppsala (Sweden) who had BST-2 transcript expression from tumor and tumor size data were considered. Patients who had tumor size values higher than 100 mm (one patient, outlier) were excluded. The publicly available GSE21422 dataset was used to determine whether there was a relationship between BST-2 expression and breast cancer classification. BST-2 expression was measured by GeneChip Robust Multiarray Averaging (GC-RMA). All data points were used. The publicly available GSE10797 dataset was used to determine whether BST-2 transcript levels are high in multiple cell types (epithelial and stromal cells) that form the tumor environment. In addition, the publicly available breast-invasive carcinoma (BRCA) data from The Cancer Genome Atlas (TCGA) data portal was used to evaluate the expression of BST-2 and patient survival. The data were downloaded through the University of Iowa's Institute for Clinical and Translational Science website [28] and through the University of California, Santa Cruz Cancer Browser. Patients who only had BST-2 expression data available from tumor tissues and not normal tissue or vice versa were excluded from the analysis of BST-2 levels in normal vs tumor tissues (100 patients were analyzed). For BST-2 level analysis in different cancer subtypes, primary tumor data was segregated on their different breast cancer subtypes and BST-2 levels were plotted. For survival analysis, primary tumor data were segregated based on BST-2 expression levels. The top 120 (highest BST-2 expressing patients—High) and bottom 120 (lowest BST-2 expressing patients—Low) samples were used for this analysis. A Kaplan-Meier plot (GraphPad Prism 6, GraphPad Software, San Diego, Calif., USA) was used to analyze survival of patients expressing different levels of BST-2 in their primary tumor tissues. Median overall survival time and area under the curve (AUC) were calculated using the GraphPad Prism 6 software.

Statistics.

Statistical analysis of significant differences was queried using the GraphPad Prism 6 software. Kaplan-Meier survival plots were analyzed with the Gehan-Breslow-Wilcoxon test using the GraphPad Prism 6 software. A probability (P) value of 0.05 or lower was considered significant.

Results

BST-2 Expression in Breast Tumor is Associated with Tumor Size, Tumor Aggressiveness, and Host Survival.

We studied BST-2 expression in different human breast cancer cells compared to normal mammary epithelial cells. Normal mammary epithelial cells did not express high BST-2, however, cancer cell lines exhibited high levels of BST-2 mRNA (Figure S1A in Additional file 1) and protein (Figure S1B in Additional file 1), consistent with a previous report [10], and suggestive of a potential role in mammary oncogenesis.

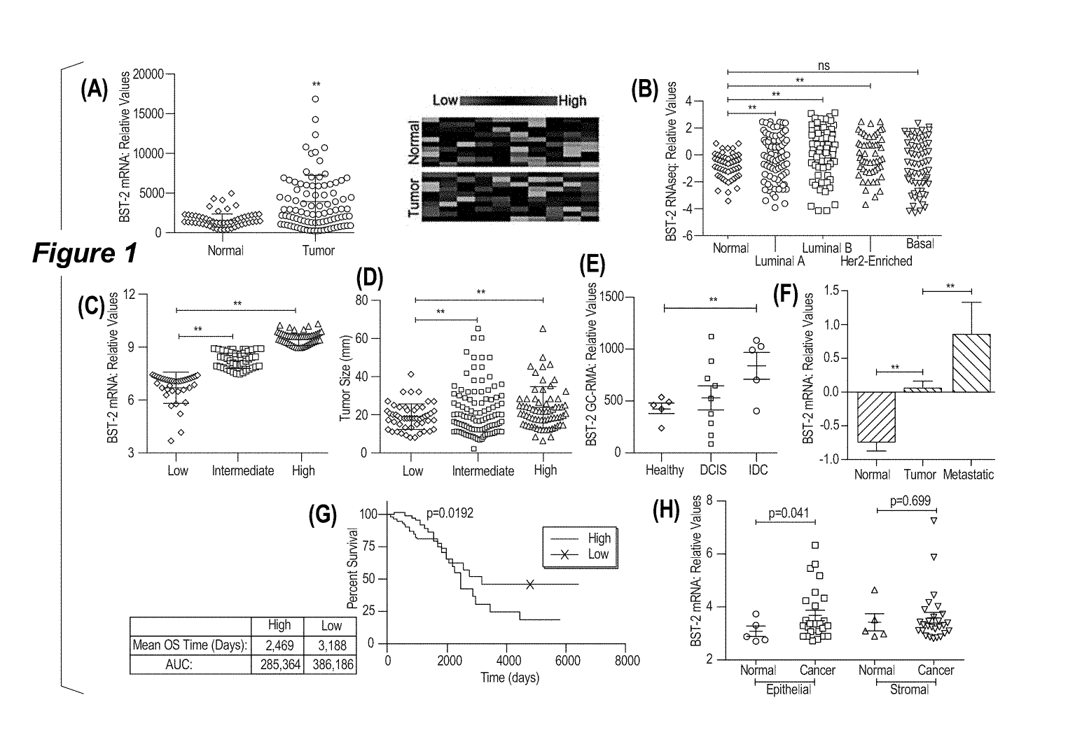

Meta-analysis of large-scale human breast cancer data from the GEO and TCGA was used to assess the level of BST-2 mRNA in breast tumors. We compared BST-2 expression in paired normal breast tissues versus resected BRCAs from subjects with known clinical outcomes. BST-2 expression was significantly higher in tumor tissues compared to their paired normal breast tissues (FIG. 1A). Stratification of TCGA data into different tumor subtypes showed that compared to normal tissues, BST-2 expression was significantly elevated in all tumor subtypes analyzed with the exception of the basal subtype, where the difference did not reach statistical significance (FIG. 1B). Of note, the high-grade luminal B tumors expressed more BST-2 mRNA than the low-grade luminal A, human epidermal growth factor receptor 2 (HER2)+, and basal type (FIG. 1B).

Large breast tumors have higher BST-2 expression compared to smaller tumors (FIGS. 1C and D) as revealed by meta-analysis of human breast cancer data using the GEO dataset GSE4922 [25]. Separation of the data into low, intermediate, and high BST-2 levels showed that subjects whose tumors had high BST-2 (FIG. 1C) had strikingly larger tumors (FIG. 1D) compared to the tumor masses in subjects with intermediate and low BST-2 (FIGS. 1C and D). These findings were consistent with the premise that high BST-2 levels may be predictive of tumor aggressiveness and reduced patient survival. We therefore investigated this possibility using the GEO dataset GSE21422 [26] containing BST-2 mRNA expression data from normal breast tissues and tumor tissues from ductal carcinoma in situ (DCIS) and invasive ductal carcinoma (IDC). As expected, BST-2 expression was higher in the most aggressive form of breast cancer, IDC, compared to DCIS (FIG. 1E).

Additionally, analysis of BST-2 expression profile with TCGA dataset segregated into normal, primary tumor, and metastatic tumor revealed that levels of BST-2 in metastatic tumors were highly elevated compared to primary tumors (FIG. 1F). Furthermore, Kaplan-Meier model showed that subjects with high tumor BST-2 had significantly reduced survival than those whose tumors had low BST-2 expression (FIG. 1G). While patients bearing high BST-2-expressing tumors had median overall survival (OS) time of 2,469 days and AUC of 285,364, subjects with low BST-2-expressing tumors had OS of 3,188 days and AUC of 386,186. These data suggest that BST-2 expression is a strong predictor of survival.

Mammary cancers are epithelial neoplasms and epithelial/stromal interactions are critical in mammary cancer development and progression. To probe into the source of BST-2 in breast tumors, the GEO dataset GSE10797 [27] was used to investigate the pattern of BST-2 expression in epithelial cells versus the surrounding stromal cells. There was no difference in BST-2 levels between stromal cells from tumor and normal mammary tissues (FIG. 1H). In contrast, BST-2 expression was significantly higher in epithelial cells from tumor compared to epithelial cells from normal breast tissues (FIG. 1H). These data suggest that epithelial cellintrinsic BST-2 may be a significant contributor of elevated BST-2 in tumor tissues. Together, these results indicate that BST-2 is most prevalent in extremely aggressive tumors and associates with patients' poor survival.

Suppression of BST-2 Expression in Mammary Cancer Cells Prolongs Time to Primary Tumor Formation and Reduces Tumor Mass.

To establish a system to analyze the functional implication of BST-2 expressed in cancer cells (Figure S2A in Additional file 2), we suppressed BST-2 expression in two murine mammary cancer epithelial cell lines, E0771 cells [29] and 4T1 cells [30]. E0771 cells are syngeneic to C57BL/6 mice while 4T1 cells are syngeneic to BALB/c mice. These models resemble human breast cancer with respect to progression and metastasis [29,30]. Using BST-2-targeting shRNA (sh137 and/or sh413), we efficiently downregulated BST-2 expression in E0771 and 4T1 cancer cells (Figures S2B to S2E in Additional file 2). A non-targeting shRNA (shControl) was used as control. Both BST-2-targeting shRNA constructs reduced BST-2 expression; but sh413 more efficiently suppressed BST-2. Consequently, sh413-expressing cells were used in all in vivo studies.

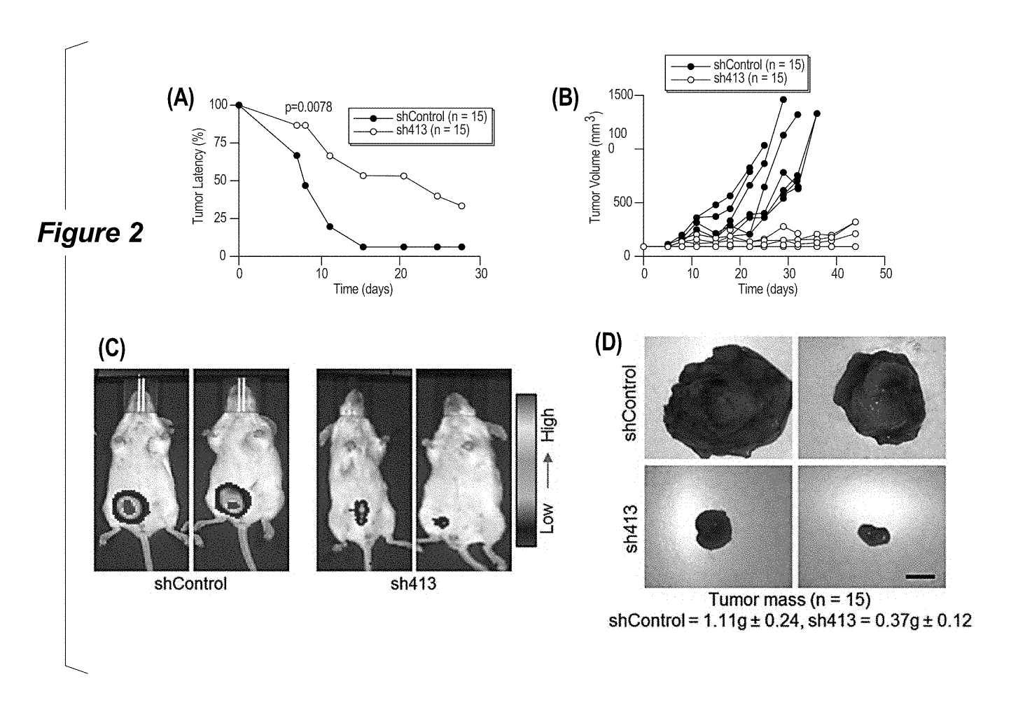

To determine the effect of BST-2 in primary mammary tumor development, we inoculated BST-2-expressing shControl and BST-2-suppressed sh413 4T1 cells into the mammary fat pads of BALB/c mice and evaluated tumor growth. 4T1 cells formed primary tumors in the mammary fat pad prior to metastasis [30]. We observed increased mammary tumor latency (FIG. 2A) and delayed mammary tumor onset (FIG. 2B) in mice implanted with BST-2-suppressed sh413 cells compared to shControl cells. Tumor volume over time was significantly lower in sh413 tumors compared to shControl tumors (FIG. 2B). Because 4T1 cells were tagged with luciferase, we tracked cancer cells in vivo by IVIS imaging. As expected, luciferase intensity (photons/sec) was much lower in mice implanted with sh413 cells compared to shControl-implanted mice at the site of injection (FIG. 2C). Inoculation of mice (n=15) with BST-2-expressing shControl cells resulted in massive mammary tumors with an average tumor mass of 1.11 g±0.24 (FIG. 2D). This result was in stark contrast to mice (n=15) inoculated with BST-2-suppressed sh413 cells that developed significantly smaller tumors averaging 0.37 g±0.12 in weight (FIG. 2D).

The effect of BST-2 in tumor development was also evident in the E0771-C57BL/6 model (Figure S3 in Additional file 3). E0771 cells are highly metastatic [29]. Expression of BST-2 in E0771 cells had a tumor-enhancing effect similar to the one observed with the 4T1 cells. BST-2-expressing E0771 cells (shControl) showed significant decrease in tumor latency compared to BST-2-suppressed E0771 cells (sh413) (Figure S3A in Additional file 3). Together, these data suggest that downregulation of BST-2 expression in breast cancer cells delays mammary tumor onset and may impair the ability of primary tumors to thrive.

Knockdown of BST-2 in Cancer Cells Decreases Metastases to the Lung and Other Distal Sites.

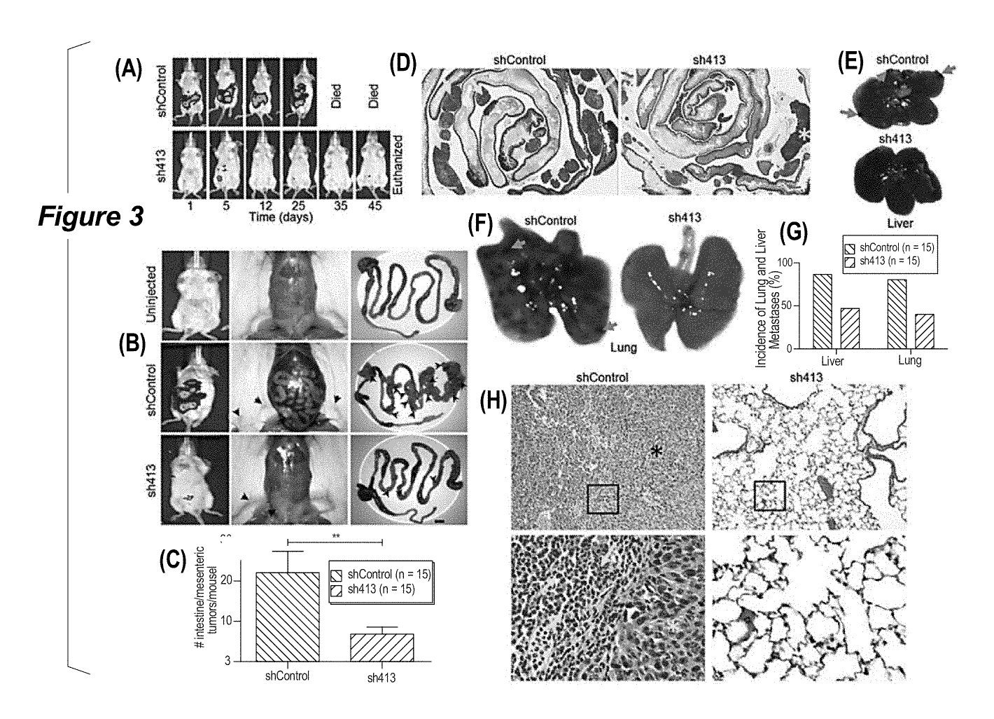

E0771 and 4T1 cells metastasize to liver, bone, lung, and intestine [29,31]. Thus, we investigated whether BST-2 enhances the metastatic potential of primary tumor cells. As expected, all mice implanted with BST-2-expressing shControl 4T1luc cells showed early onset and progressive increase in bioluminescence. The increase in bioluminescence signal intensity over time suggests progression and metastasis of cancer (FIG. 3A, upper panel, FIG. 3B, left, middle panel). Indeed, BST-2-expressing shControl cells formed primary tumors quickly and developed metastatic lesions that could be detected by bioluminescence imaging [32]. In striking contrast, BST-2-suppressed 4T1 cells (sh413) exhibited delayed onset of luciferase bioluminescence and disappearance of expression as measured over 45 days (FIG. 3A, lower panel; FIG. 3B, left, bottom panel). Unlike shControl-implanted mice that developed severe abdominal hemorrhage and intestinal/mesenteric tumors (FIG. 3B, center, middle and right panels), sh413-implanted mice did not develop hemorrhage and had few intestinal/mesenteric tumors (compare FIG. 3B, uninjected—upper panel with FIG. 3B, sh413-injected—lower panel). Metastasis to the intestine and mesentery were significantly reduced from about 21 tumors in shControl mice (FIG. 3B, middle right panel and FIG. 3C) to six tumors in sh413 mice (FIG. 3B, bottom right panel and FIG. 3C). Histology confirmed increased intestinal/mesentery tumors in shControl-implanted mice compared to sh413-implanted mice (FIG. 3D, arrows). These findings were confirmed with the highly metastatic E0771 cells. Mice (n=10) implanted with BST-2-expressing E0771 cells (shControl) had higher bioluminescence and increased intestinal/mesenteric tumors compared to mice implanted with BST-2-suppressed sh413 cells (Figures S3B to S3D in Additional file 3).

Importantly, gross images showed that compared to sh413 cells, shControl cells resulted in significant metastases to the liver (FIG. 3E) and lung (FIG. 3F). Lungs from shControl-implanted mice were laden with pulmonary nodules, suggesting pulmonary metastases (FIG. 3F, arrows). The incidence of metastases to the liver was 86% and 46%, and 80% and 40% to the lung for 4T1 shControl and sh413 cells respectively (FIG. 3G). Similar trends in liver and lung metastases were observed with E0771 cells (Figure S3E in Additional file 3). Furthermore, histologic analyses confirmed large metastatic nodules in the lung of mice implanted with 4T1 shControl cells (FIG. 3H, asterisk). Additionally, there were clustered and scattered tumor cells throughout the lung interstitium with marked infiltration of neutrophils within the alveolar septa and alveolar spaces of lungs from shControl-injected mice (FIG. 3H, arrows).

To test whether the reduced metastasis observed in mice bearing tumors from BST-2-suppressed cells reflect a delay in metastasis due to delayed primary tumor growth and differences in tumor size, we performed a linear regression analysis for correlation between primary tumors and metastatic growth. However, we found no correlation between primary tumor and lung or primary tumor and intestinal/mesentery metastases in our mouse models (not shown). These results show that BST-2 expression promotes mammary tumor metastasis to distal sites.

BST-2 Expression in Mammary Cancer Cells is Associated with Poor Clinical Outcome and Significant Morbidity in Tumor-Bearing Mice.

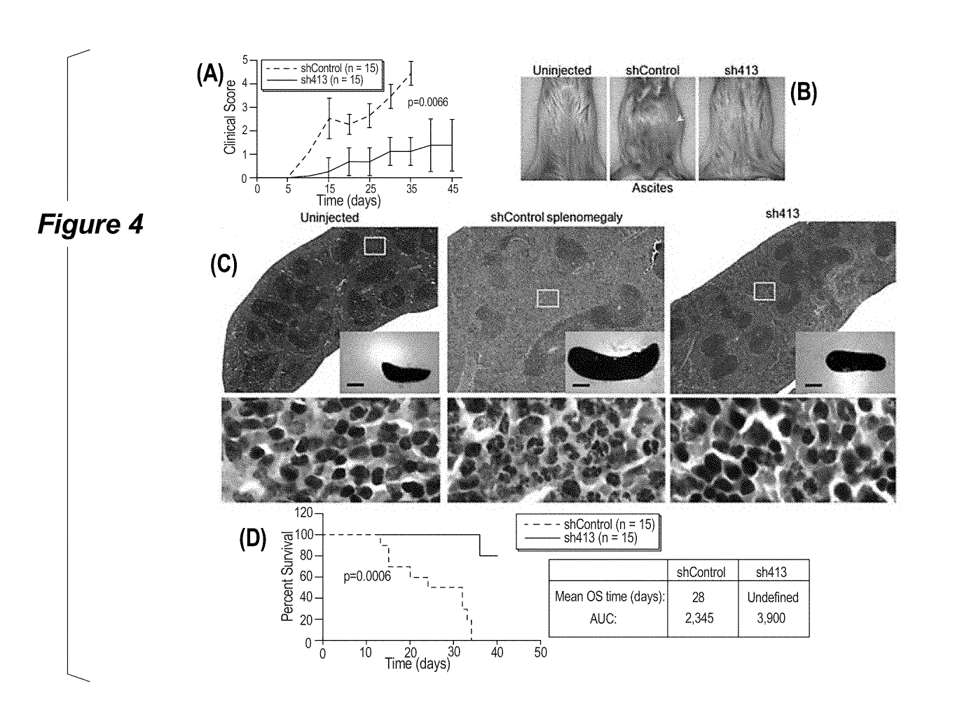

Pronounced effect on morbidity was observed in mice bearing shControl-induced tumors compared to their counterparts bearing sh413-induced tumors. Specifically, mice implanted with BST-2-expressing 4T1 cells developed hypothermia more rapidly and to a higher extent than mice implanted with BST-2-suppressed sh413. Ruffled hair, shallow breathing, and prostration were observed in shControl-implanted mice but not in sh413-implanted mice (FIG. 4A). Furthermore, mice implanted with BST-2-expressing shControl 4T1 cells developed malignant ascites (FIG. 4B, middle panel) and severe splenomegaly (FIG. 4C, middle panel, inset). Remarkably, 14 out of 15 mice implanted with BST-2-suppressed 4T1 cells (sh413) were spared of ascites (FIG. 4B, compare left and right panels) and splenomegaly (FIG. 4C, compare left and right panel insets). Grossly, spleens from shControl mice were markedly enlarged (FIG. 4C, inset). Histologically, the splenic red pulp of shControl-implanted mice was markedly expanded by increased immature and mature granulocytes indicative of increased granulopoiesis (FIG. 4C, shControl, lower panel). In contrast, there was a slight increase in red pulp granulocytes in the spleen of sh413-bearing mice (FIG. 4C, sh413, lower panel).

Similar to the 4T1 model, clinical manifestations of disease were delayed in BST-2-suppressed sh413 E0771-bearing mice (Figure S4A in Additional file 4). Suppression of BST-2 in E0771 cells prevented the development of malignant ascites in all (n=10) sh413 bearing mice compared to shControl-bearing mice (Figure S4B, upper panel in Additional file 4). Moreover, BST-2-suppressed E0771 (sh413)-bearing mice did not develop shock (assessed by the appearance of pale digits on forelimbs) as was observed in all E0771 shControl-bearing mice (Figure S4B, lower panel in Additional file 4) and as previously shown in the E0771 model [29]. These results show that expression of BST-2 in cancer cells accelerates disease progression in tumor-bearing mice.

BST-2-Expression in Cancer Cells Results in Poorer Survival of Tumor-Bearing Mice.

Because human breast cancer patients bearing tumors with high BST-2 mRNA have lower survival, we directly evaluated the role of BST-2 expression in cancer cells on the survival of tumor-bearing mice. Kaplan-Meier survival curve analysis reveals that mice implanted with BST-2-suppressed sh413 4T1 or E0771 cells have a statistically significant prolongation in survival compared with BST-2-expressing shControl-implanted mice (FIG. 4D (4T1) and Figure S4C (E0771) in Additional file 4). Improvement in survival was more pronounced in the 4T1 model because all (n=15) mice implanted with 4T1 shControl cells died on average 25 days post implantation. Surprisingly, 14 out of 15 mice implanted with 4T1 sh413 cells survived and were euthanized at the end of the experiment (day 45). One out of 15 4T1 sh413-implanted mice was sacrificed on day 37 post implantation due to tumor-associated morbidity (FIG. 4D). The OS and AUC for 4T1 shControl-bearing mice were 28 days and 2,345 compared to sh413-bearing mice with undefined OS and 3,900 AUC. Additionally, mice implanted with E0771 shControl cells died at approximately 16 days post implantation compared to their E0771 sh413 cells-implanted counterparts that averaged 23 days post implantation (Figure S4C in Additional file 4). The OS and AUC of E0771 shControl mice were 16 days and 1,965 respectively, while E0771 sh413-implanted mice have 23 days OS and 2,679 AUC. Together with the human survival data presented in FIG. 1G, our results support the premise that BST-2 expression in mammary cancer cells may be a predictor of host survival.

Intrinsic BST-2 in Mammary Cancer Epithelial Cells Modulates Cancer Cells Adhesion.

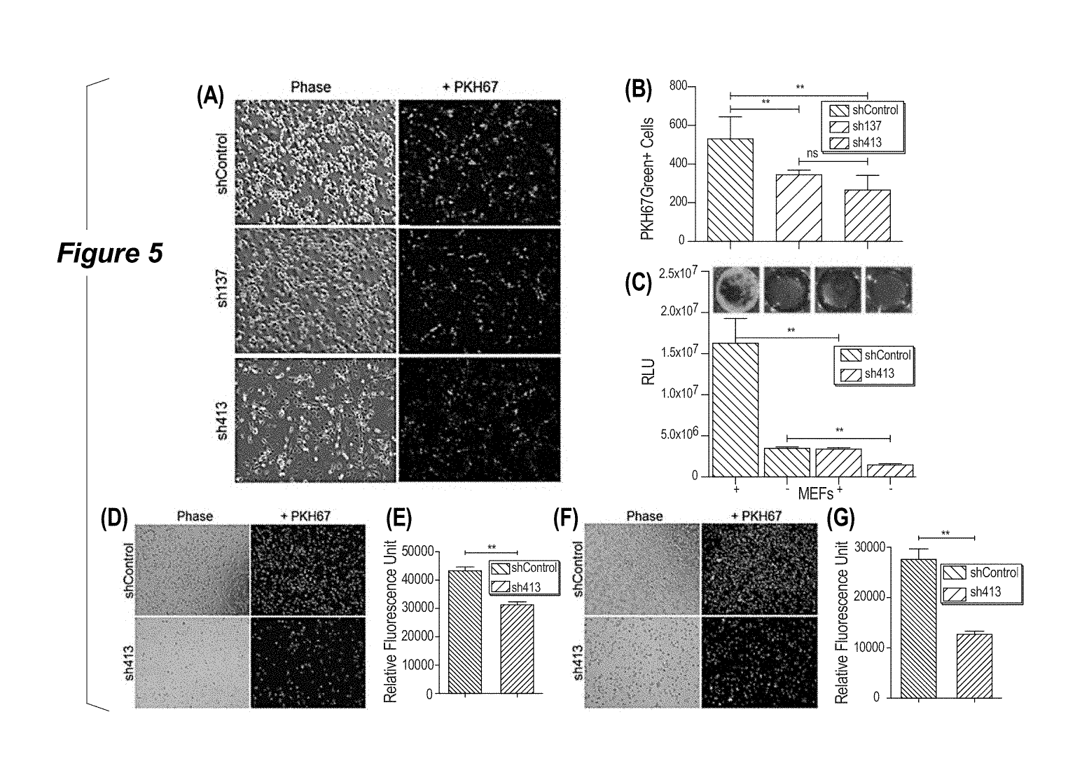

The striking effects of BST-2 on tumor growth and metastasis led us to define the cellular basis for BST-2 effect on breast tumorigenesis. One characteristic feature of cancer cells is their ability to adhere to and recruit other cells, such as cancer-associated fibroblasts (CAFs) to promote formation of primary tumors [33]. To determine the role of BST-2 in cancer cell adhesion, E0771 cells with varying BST-2 levels were labeled with the fluorescent cell linker PKH67Green dye and added onto confluent monolayers of MEF. We found that cancer cell BST-2 facilitated cancer cell adhesion to fibroblasts as revealed by confocal microscopy (FIG. 5A) and Image J quantification (FIG. 5B) of PKH67Green+ cells adhered to MEFs. Large numbers of the BST-2-expressing (shControl) adhered to fibroblasts compared to cells with intermediate BST-2 (sh137) and low BST-2 (sh413) respectively. In parallel, a similar experiment was performed with luciferase expressing shControl or sh413 cancer cells added onto confluent MEFs. Consistent with the PKH67Green result, suppression of BST-2 decreased the ability of cancer cells to adhere to supporting cells. Compared to shControl cells, lower luciferase bioluminescence was observed in wells containing sh413 both in a luciferase assay (FIG. 5C) and by IVIS 200 imaging of luciferase bioluminescence (FIG. 5C, inset). To confirm the adhesion result, we analyzed the effects of BST-2 knockdown on adhesion to the extracellular matrix (ECM) supportive substrates collagen and fibronectin [34] using pre-coated plates. As expected, BST-2-suppressed sh413-expressing 4T1 cells had reduced adhesive capability to collagen (FIGS. 5D and E) and fibronectin (FIGS. 5F and G) compared to BST-2-expressing shControl cells. These results indicate that BST-2 regulates adhesion of breast cancer cells to CAFs and to ECM proteins.

BST-2 Depletion Reduces Anchorage-Independent Growth.

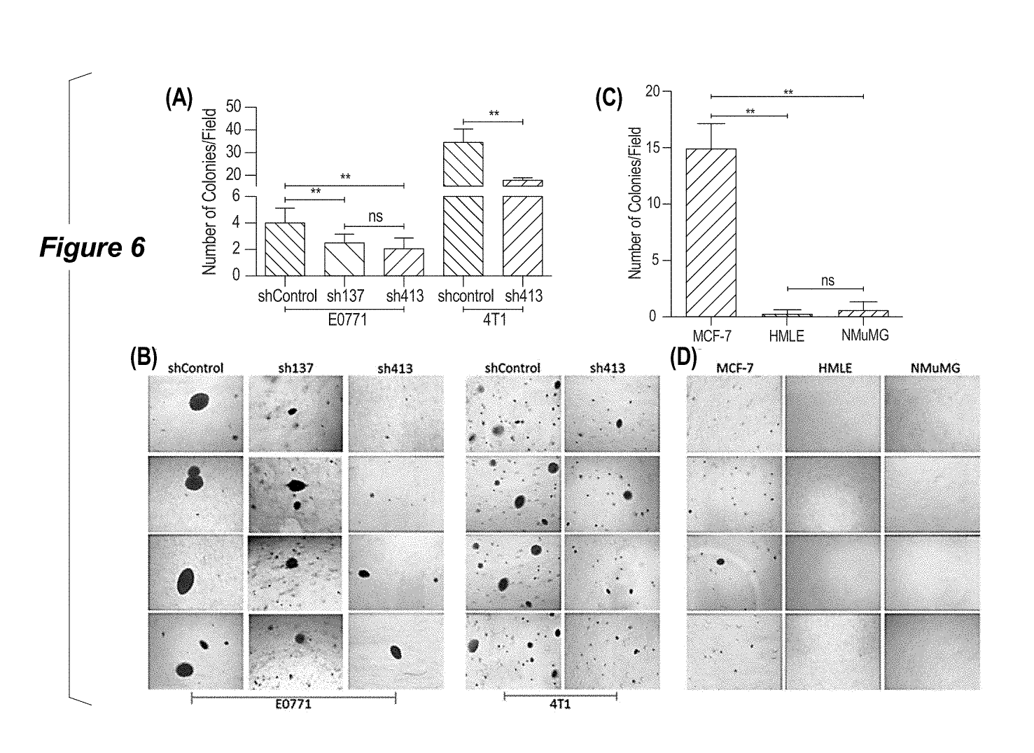

Adaptation to new environment is a hallmark of aggressive tumors. To survive, cancer cells are able to grow and expand in the absence of attachments by overcoming anoikis [35]. Because BST-2-expressing shControl cells metastasized more efficiently than BST-2-suppressed sh413 cells in vivo (FIG. 3), we used a soft agar colony formation assay to examine the possibility that BST-2 is important for anchorage-independent growth of mammary cancer cells. As expected, we observed reduced colony numbers (FIG. 6A) and colony size (FIG. 6B) in mammary cancer cells with suppressed BST-2 (intermediate-sh137 and low-sh413) compared to cells expressing high BST-2 (shControl). MCF-7 cells, known to form colonies [36,37], were used as positive control (FIGS. 6C and D) while normal human (HMLE) and murine (NMuMG) mammary epithelial cells were used as negative controls (FIGS. 6C and D). Interestingly, overexpression of BST-2 in low BST-2-expressing MCF-7 cells (Figure S5A in Additional file 5) increased MCF-7 colony size relative to empty vector control (Figure S5B and S5C in Additional file 5). These data suggest that suppression of BST-2 expression may diminish in vivo tumorigenicity of otherwise highly tumorigenic cancer cells by reducing anchorage-independence of tumor cells, thus preventing expansion of tumor cells, invasion to adjacent tissues, and dissemination throughout the body.

BST-2 Expression Promotes Cancer Cell Migration and Invasion.

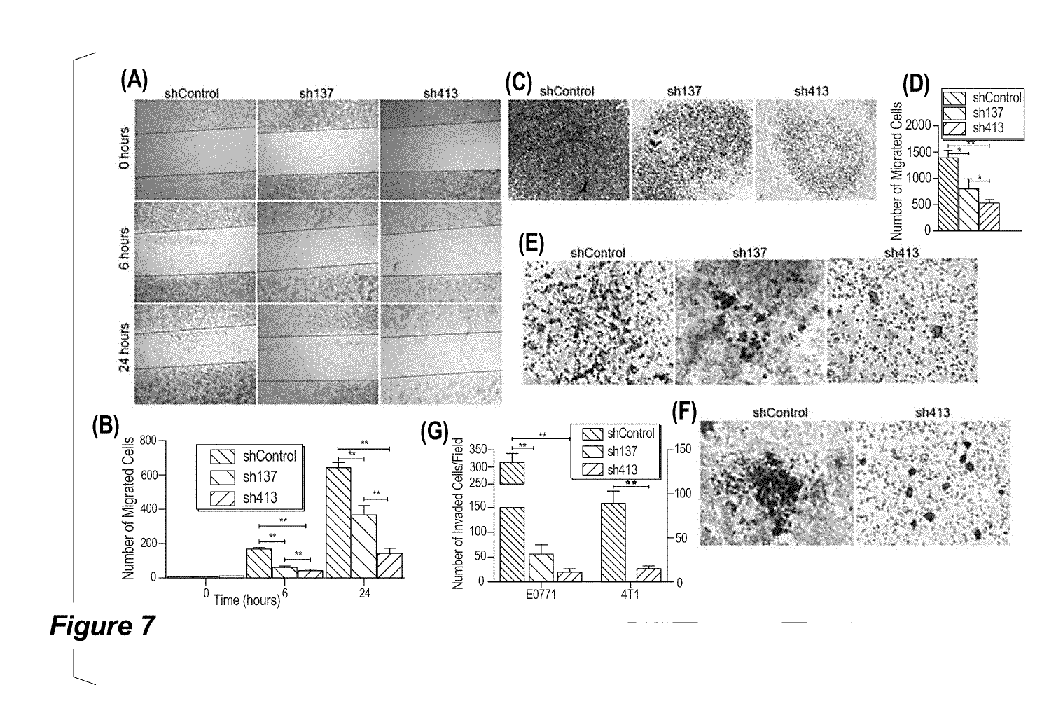

Following the formation of primary tumors, a subpopulation of cancer cells acquires a metastatic phenotype that allows them to migrate to distant tissues [38]. Our in vivo study revealed that expression of BST-2 may promote tumor growth at secondary sites (FIG. 3). Because cancer cell migration and invasion are key to metastasis, including dissemination of tumor cell into the lymphatic and blood vessels, and subsequent extravasation of tumor cells into secondary organs [39,40], we evaluated the effect of BST-2 on cancer cells migration using classical migration scratch assay with the cell comb scratch assay. Cancer cells suppressed of BST-2 (sh137 and sh413) lost their ability to migrate to the scratched wounds compared with those expressing BST-2 (shControl) at both 6 h and 24 h time points (FIGS. 7A and B). The rate of cell migration into the wound opening was reduced in line with level of BST-2 expression, thus, sh137 cells showed some migration (FIG. 7A, column 2, FIG. 7B) while sh413 cells lost the ability to migrate (FIG. 7A, column 3, FIG. 7B). The rate of migration was quantified by blind-counting of at least five different fields. In parallel, we performed migration assay on MCF-7 cells overexpressing BST-2. MCF-7 cells overexpressing BST-2 had higher migratory rate compared to vector control cells (Figure S5D in Additional file 5). These results show that the rate of cancer cell migration is strongly associated with BST-2 levels.

To further evaluate the role of BST-2 in cancer cell migration, we employed commercially available Boyden chamber assays. Equal numbers of shControl, sh137, and sh413-expressing cells were plated in the apical chamber of cell culture inserts. Serum-containing medium was added to the basolateral chamber. Compared to cells with high BST-2 expression (shControl), suppression of BST-2 with sh137 and sh413 significantly reduced rate of cell migration (FIGS. 7C and D).

Although BST-2 increased rate of cell migration into the scratch wound, we found no BST-2-dependent difference in rate of wound closure at 6 h and 24 h time points, suggesting that cells with high (shControl) and suppressed BST-2 (intermediate-sh137 and low-sh413) expression may proliferate equally. Indeed, proliferation assay examining rate of BrdU incorporation into cells showed that endogenous BST-2 had no effect on cell proliferation (Figure S6A and S6B in Additional file 6). This result is in contrast with a previous study that showed that exogenous overexpression of BST-2 promotes cell proliferation [21]. It is likely that the differences in results are due to different experimental systems or cells. In our study, the lack of BST-2 effect on cell proliferation, upon BST-2 knockdown, may not be due to cell viability because metabolic activity of MTT revealed that both BST-2-expressing and BST-2-suppressed E0771 cells were equally viable (Figure S6C in Additional file 6). However, in 4T1 cells, BST-2 knockdown increased cell viability (Figure S6D in Additional file 6). These data suggest that the effects of BST-2 in colony formation and migration (FIGS. 6 and 7, respectively) cannot be explained by differences in cell viability or cell proliferation in vitro. Although we did not observe BST-2-endowed growth advantage in our two-dimensional culture, we cannot rule out the possibility that BST-2 may promote cell proliferation in soft agar or in vivo.

In order to metastasize, cancer cells have to migrate and invade the basement membrane. Hence we investigated the ability of BST-2 to promote cancer cell invasion using a Matrigel model. BST-2-expressing (shControl) and BST-2-suppressed (intermediate-sh137 and low-sh413) cells were allowed to invade into the Matrigel for 24 h. As shown in FIGS. 7E, F, and G, significantly higher numbers of BST-2-expressing shControl cells invaded into the Matrigel compared to BST-2-suppressed sh137 and sh413 cells in that order. An enhancement in cancer cell invasion also resulted when BST-2 was overexpressed in MCF-7 cells (Figure S5E in Additional file 5). These experiments demonstrate that BST-2 expression is crucial for cancer cell invasion and that suppressing BST-2 expression in cancer cells reduced the ability of the cells to invade the basement membrane.

### Discussion

Host innate immune response is critical for surveillance against pathogens and tumors. However, genes involved in immune response may serve as a double-edged sword in pathogenesis and tumorigenesis. As an innate immunity antiviral gene, BST-2 positively regulates NF-κB activation [14,15] and its expression is induced by types I and II interferons [16]. Increased expression of BST-2 retains budding viruses to the cell plasma membrane [15,16] and inhibits virus replication [17,41]. However, elevated levels of BST-2 in cancer cells have pro-tumor functions [10,20]. In this study, we demonstrated that BST-2 expressed in cancer cells promoted breast cancer development and progression by altering the behavior of cancer cells. Meta-analysis of TCGA (BRCA) human data that showed that BST-2 is most significantly associated with luminal B tumors, invasive ductal carcinoma, and metastatic tumors imply that BST-2 in cancer cells could be a prognostic factor for highly aggressive cancers. It is known that luminal B tumors are associated with larger tumor mass [42] and patients bearing this tumor subtype have significantly worse disease-free survival compared to patients with luminal A tumors [43]. In our meta-analysis study, we found that human breast tumors with elevated BST-2 mRNA are larger, more aggressive, and patients bearing such tumors have poorer survival. This association study was validated in our mouse model experiments.

Mouse models have contributed to understanding breast oncogenesis [30]. In our studies, we used two syngeneic mouse models to allow investigation of the contribution of cancer cell BST-2 in mammary tumorigenesis in different backgrounds in the context of an intact immune system. Implantation of BST-2-expressing 4T1 or E0771 cells into syngeneic BALB/c or C57BL/6 mice respectively revealed that BST-2 in cancer cells is disease modifying. However, suppressing BST-2 expression decreased the onset of primary mammary tumor growth thereby increasing tumor latency, and decreasing tumor cell metastases and growth at distal sites, as in lung colonization.

Whether the decrease in metastasis observed in mice bearing tumors from BST-2 suppressed cells is a direct result of reduced tumor size or delayed metastasis is unknown. However, our in vitro studies that showed that cells with suppressed BST-2 have reduced adhesion, anchorage-independent growth, migration, and invasion supports a role for BST-2 in promoting tumor growth at distal sites, because these cancer cell behaviors are critical for metastasis [39,40]. In addition, the lack of correlation between formation of primary tumor and lung or intestinal/mesentery colonization in our mouse model suggests that BST-2 may differentially promote tumor growth at the primary and secondary sites.

In addition to increased tumor growth at the primary and secondary sites, mice bearing BST-2-expressing 4T1 shControl cells developed malignant ascites and splenomegaly or ascites and shock in the case of E0771 shControl cells-bearing mice. Ascites in tumor-bearing mice may result from the accumulation of fluid in the peritoneal cavity due to the spread of cancer cells [44]. Ascites is associated with increased vessel permeability and decreased lymphatic drainage [45]. Indeed, human patients with cancer-associated ascites have poor prognosis [46]. It is intriguing that BST-2-suppressed sh413-injected mice had a delayed occurrence (E0771 cells) or absence (4T1 cells) of ascites.

Aside from ascites, mice implanted with 4T1 BST-2-expressing cells but not BST-2-suppressed cells developed severe splenomegaly with expanded splenic red pulp, suggestive of increased granulopoiesis. In mice, the spleen is a normal site of hematopoiesis and reactive hematopoiesis. Thus, the splenic granulocytic hyperplasia in shControl mice is the result of reactive hematopoiesis secondary to granulocyte recruitment to the site of tumor. In humans, splenomegaly can be the result of extramedullary hematopoiesis (the spleen is not a normal site of hematopoiesis in humans) but more commonly a result of cancer cell metastases due to hematogenous disease [47]. Malignant ascites and splenomegaly are manifestations of end-stage events in many cancers including breast cancers [48,49] and is linked to poor prognosis in tumor-bearing hosts.

BST-2 expression in tumor tissues is positively associated with hosts' survival. In mice, tumors induced by BST-2-expressing shControl cells were associated with poor survival. The observed difference in survival between 4T1 cells and E0771 cells models could be attributed to (i) the level of aggressiveness of the cells; with E0771 cells being more metastatic [29] than 4T1 cells [30], and (ii) the level of BST-2 in the different cancer cells. We found that, BST-2 expression increased with tumor aggressiveness in human patients. BST-2 expression was highest in the highly aggressive IDC compared to DCIS tumors. Moreover, metastatic tumors expressed more BST-2 than primary tumors.

Although the source of elevated BST-2 in breast tumors is unknown, our data suggest that BST-2 expression in breast epithelial cells derived from breast tumors was significantly higher than BST-2 in normal breast epithelial cells. However, BST-2 expression between stromal cells (tumors versus normal breast tissues) was not different, indicating that tumor epithelial cells could partly be contributory to elevated BST-2 in tumor tissues. Therefore, BST-2 upregulation may be an important step in a series of changes that tumor cells undergo during transformation.

Intriguingly, we found that the cellular mechanisms responsible for the tumorigenic potential of BST-2 include alterations in cancer cell adhesion, anchorage-independency, migration, and invasion, but not proliferation. In two dimensional culture, suppression of BST-2 in murine cancer cells had no effect on cell proliferation despite decreased ability of these cells to grow independent of anchor. Although not tested in this study, it is possible that suppression of BST-2 may result in decreased proliferation and increased susceptibility to apoptosis in vivo. However, in two-dimensional culture, we found that 4T1 cells but not E0771 cells with suppressed BST-2 expression have higher viability as measured by MTT assay, indicating that cell viability may not be implicated in the role of BST-2 in cancer cell behavior. The attribute of BST-2 that endows it the ability to simultaneously promote so many different malignant processes is yet to be discovered.

It is possible that the expression of BST-2 provides cancer cells a suitable milieu for their growth and spread through the following processes: (i) alteration of cancer cell stiffness enhancing cancer cell adhesion to extracellular matrix and escape from primary tumor [50,51]; (ii) heightened NF-κB activity and conversion of NF-κB-induced inflammatory stimuli into tumor growth and metastatic signals; (iii) promotion of cancer cells secretion of soluble signaling molecules that potentiate tumor growth and metastasis; and (iv) synthesis of endopeptidases such as matrix metalloproteinases to facilitate degradation of various components of the extracellular matrix, thereby promoting tumorigenesis. Further investigations are required in this respect.

Conclusions

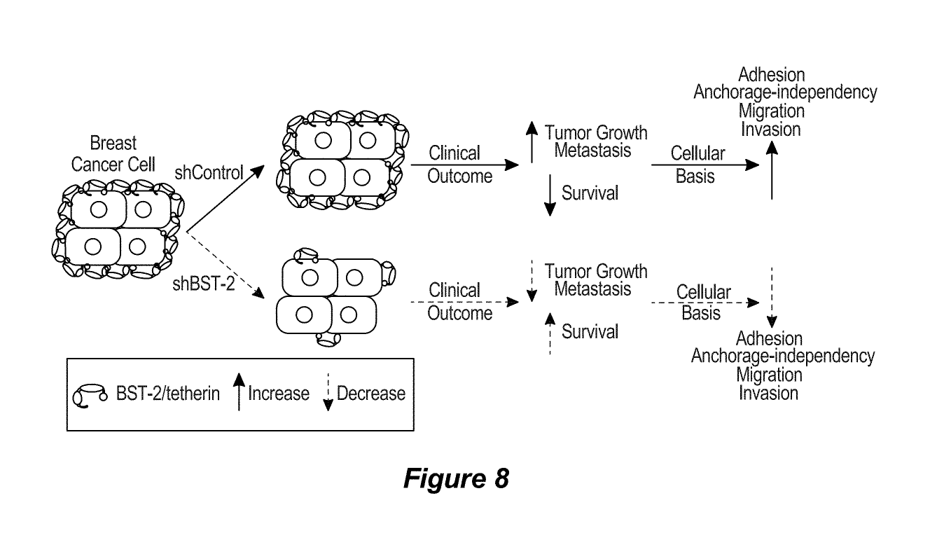

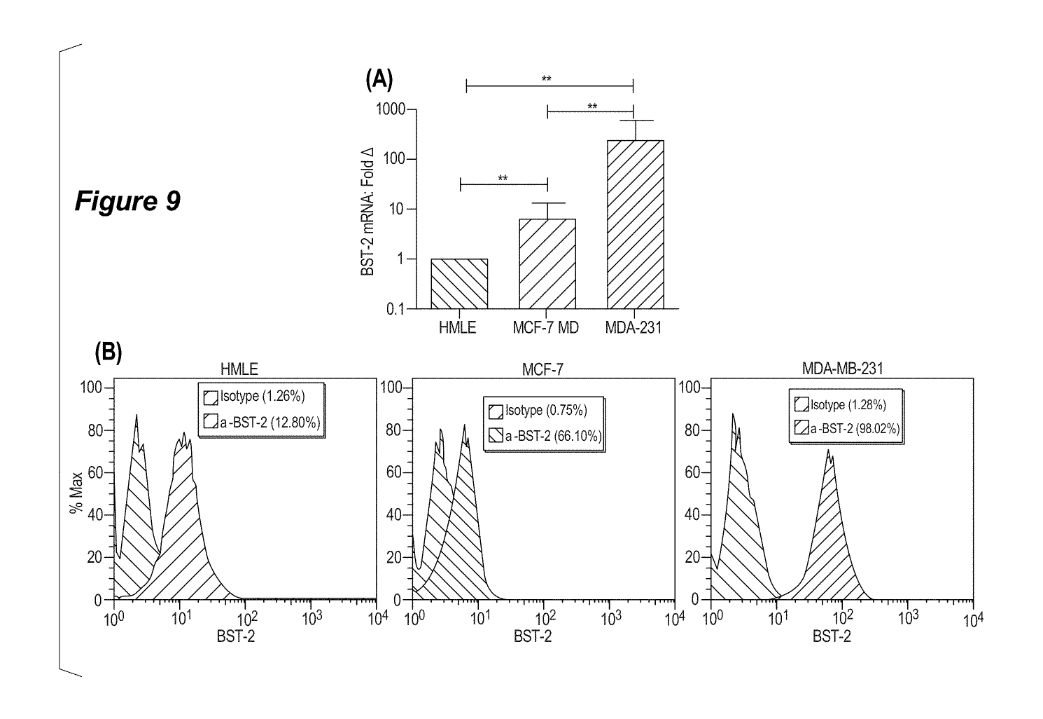

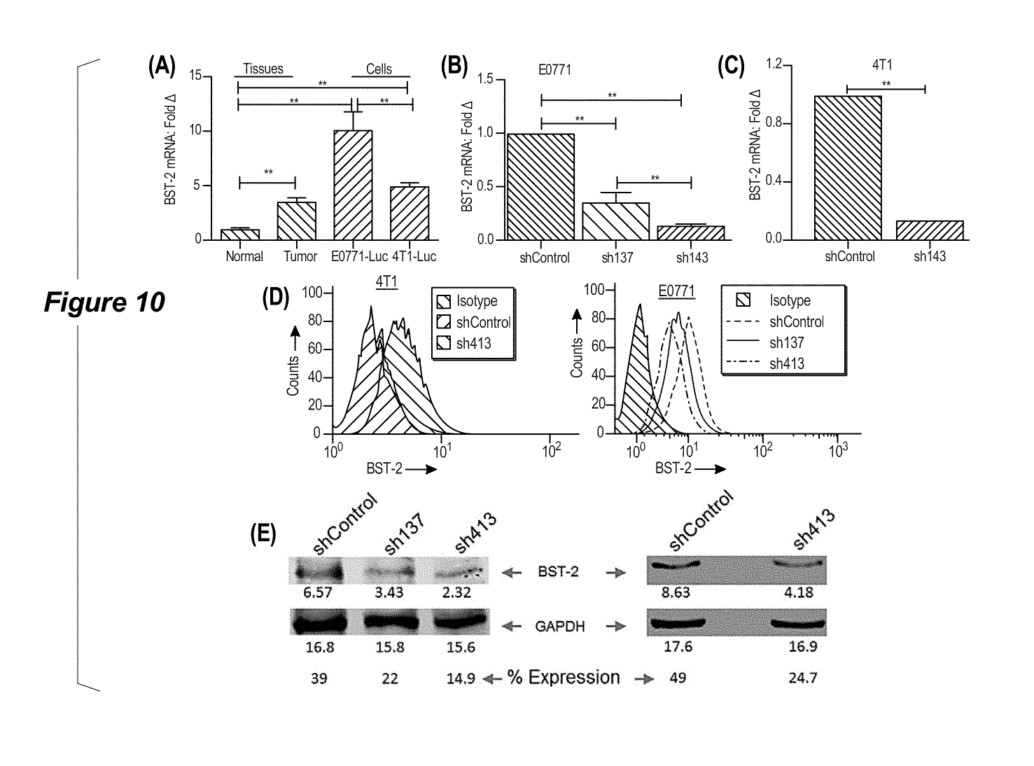

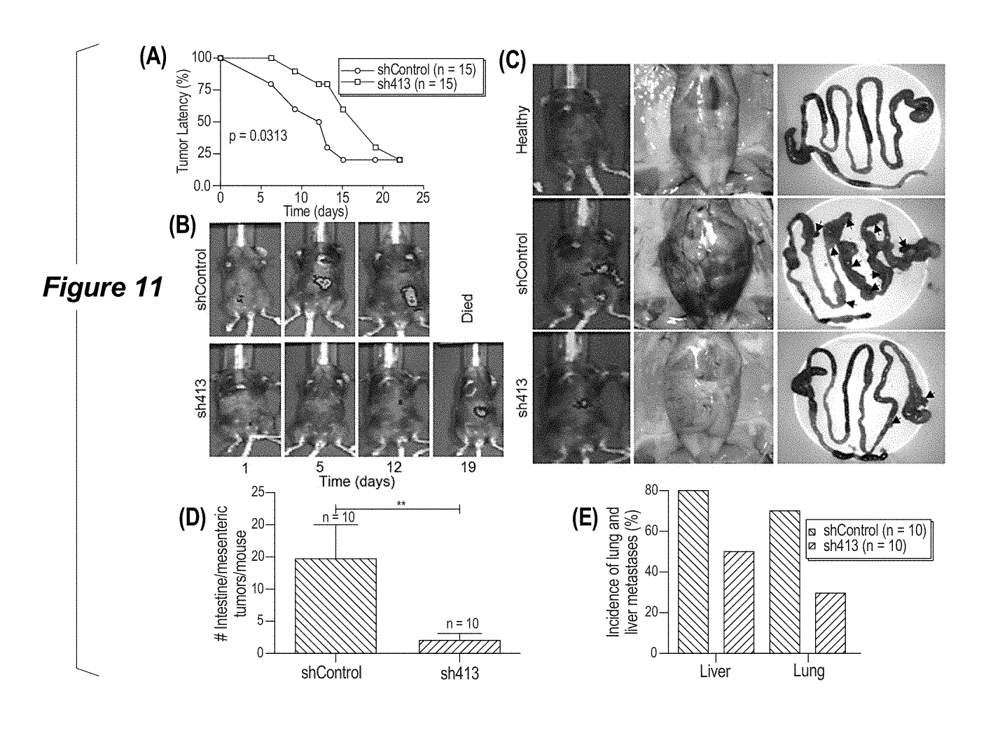

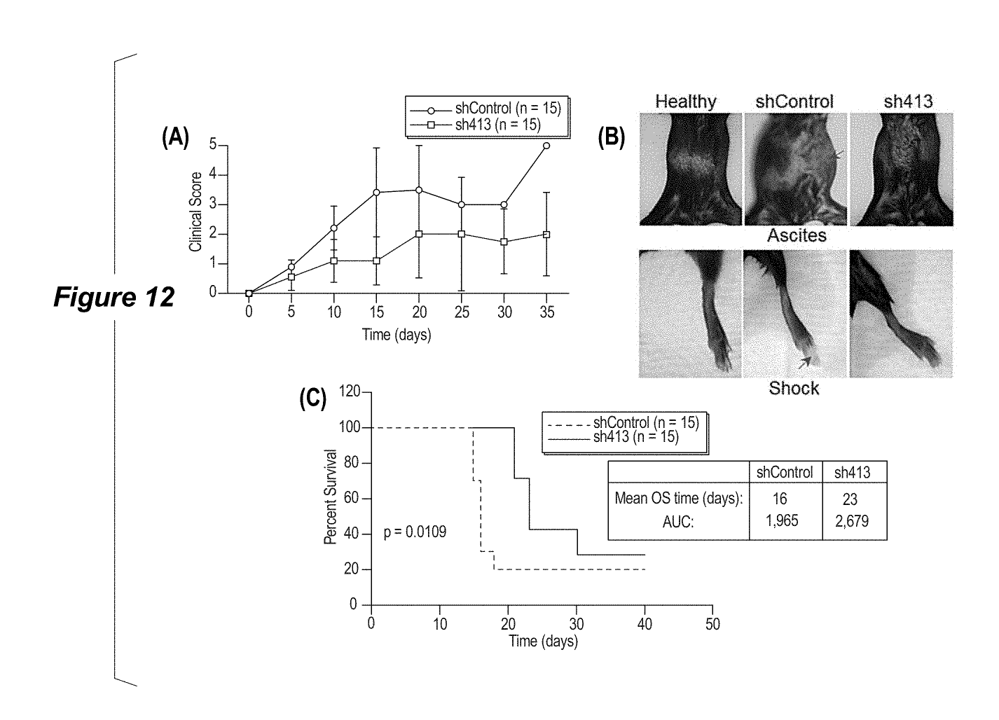

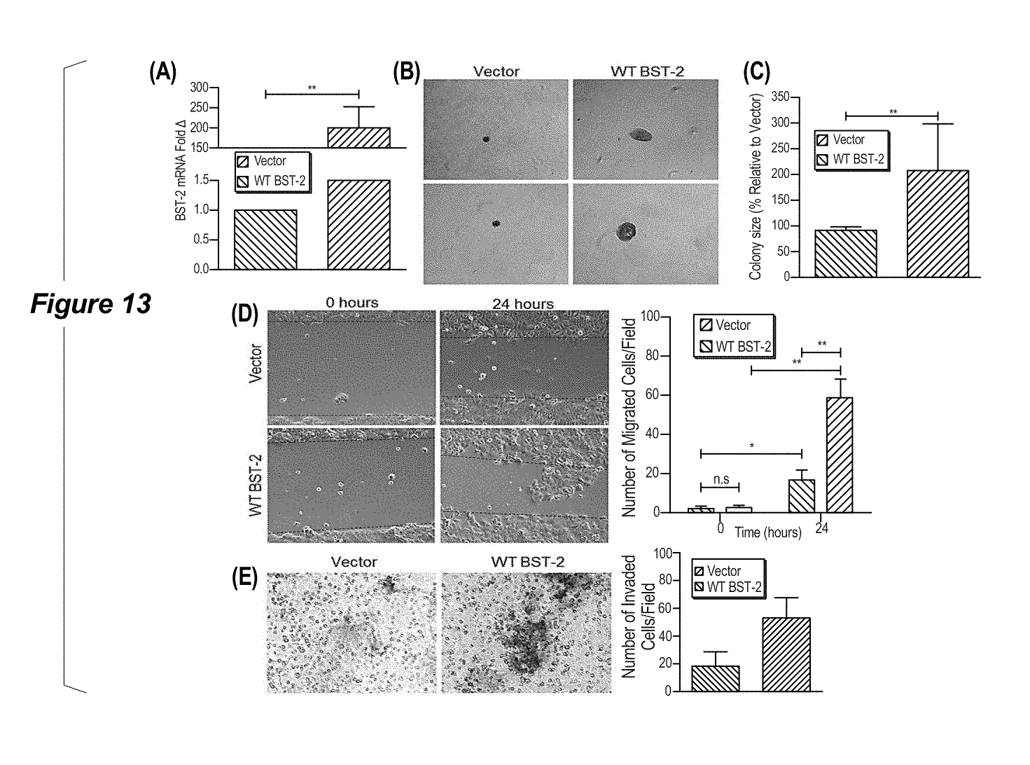

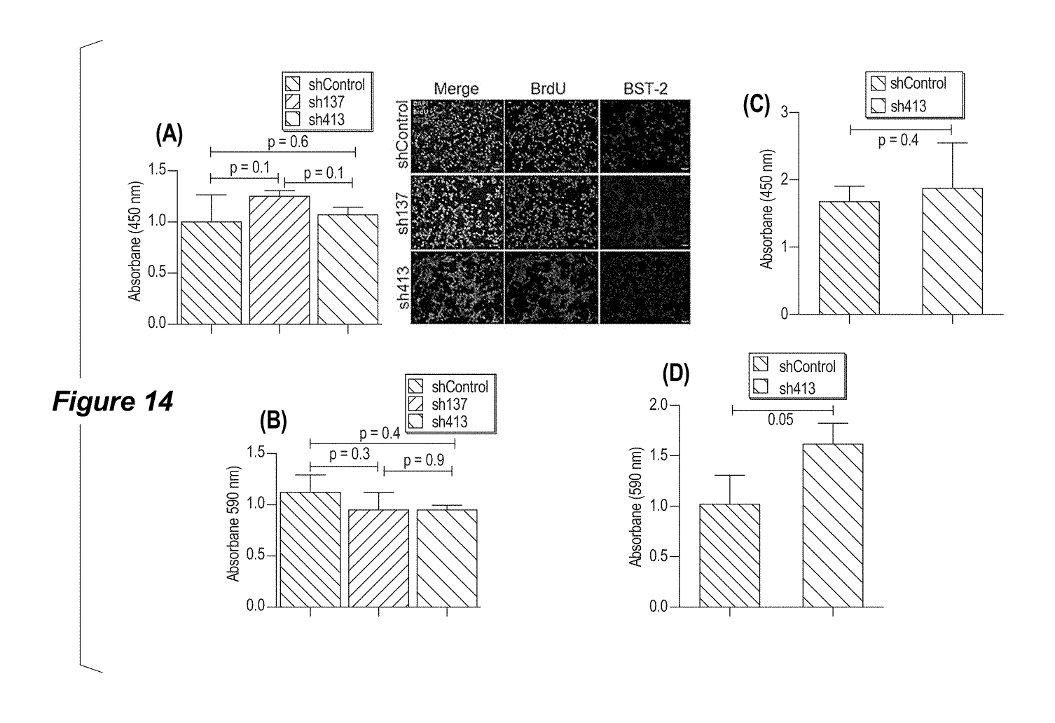

The results of this study as summarized in our model (FIG. 8) reveal the critical role of BST-2 in the many processes involved in mammary oncogenesis by showing that BST-2 expressed in carcinoma cells is a positive disease modifier and elevated levels of BST-2 predict tumor aggressiveness and host survival. The inability of mammary cancer cells with suppressed BST-2 to efficiently colonize independent of attachment, along with the observed increase in tumor latency in mice implanted with BST-2-suppressed cells suggest that BST-2 action may be early and sustained in the process of mammary tumorigenesis. This report highlights the importance of cell-intrinsic BST-2 in the emergence of neoplasia and malignant progression of breast cancer. Therefore, BST-2 may serve as a biomarker for aggressive breast cancers and as a potential target for the development of new therapeutics for BST-2-dependent cancers.

### Abbreviations

7-AAD: 7-aminoactinomycin D; APC: allophycocyanin; AUC: area under the curve; BRCA: breast-invasive carcinoma; BrdU: bromodeoxyuridine or 5-bromo-2′-deoxyuridine; BSA: bovine serum albumin; BST-2: bone marrow stromal antigen 2; CAFs: cancer-associated fibroblasts; CD317: cluster of differentiation 317; DAPI: 4′,6-diamidino-2-phenylindole; DCIS: ductal carcinoma in situ; ECM: extracellular matrix; FACS: fluorescence-activated cell sorting; FBS: fetal bovine serum; FITC: fluorescein isothiocyanate; GEO: Gene Expression Omnibus; H&E: hematoxylin and eosin; HER2: human epidermal growth factor receptor 2; IDC: invasive ductal carcinoma; IgG: immunoglobulin G; MEF: murine embryonic fibroblast; NF-κB: nuclear factor kappa binding; OS: overall survival; PBS: phosphate-buffered saline; PFA: paraformaldehyde; RPMI: Roswell Park Memorial Institute medium; shRNA: short hairpin RNA; TCGA: The Cancer Genome Atlas; TV: tumor volume.

### REFERENCES FOR EXAMPLE 1

1. Balmain A, Gray J, Ponder B: The genetics and genomics of cancer. Nat Genet 2003, 33(Suppl):238-244.
2. Tu S, Bhagat G, Cui G, Takaishi S, Kurt-Jones E A, Rickman B, Betz K S, Penz-Oesterreicher M, Bjorkdahl O, Fox J G, Wang T C: Overexpression of interleukin-1beta induces gastric inflammation and cancer and mobilizes myeloid-derived suppressor cells in mice. Cancer Cell 2008, 14:408-419.
3. Lin E Y, Nguyen A V, Russell R G, Pollard J W: Colony-stimulating factor 1 promotes progression of mammary tumors to malignancy. J Exp Med 2001, 193:727-740.
4. Wang W, Nishioka Y, Ozaki S, Jalili A, Abe S, Kakiuchi S, Kishuku M, Minakuchi K, Matsumoto T, Sone S: HM1.24 (CD317) is a novel target against lung cancer for immunotherapy using anti-HM1.24 antibody. Cancer Immunol Immunother 2009, 58:967-976.
5. Silveira N J, Varuzza L, Machado-Lima A, Lauretto M S, Pinheiro D G, Rodrigues R V, Severino P, Nobrega F G, Silva W A Jr, De B, Pereira C A, Tajara E H: Searching for molecular markers in head and neck squamous cell carcinomas (HNSCC) by statistical and bioinformatic analysis of larynx-derived SAGE libraries. BMC Med Genomics 2008, 1:56.
6. Fang K H, Kao H K, Chi L M, Liang Y, Liu S C, Hseuh C, Liao C T, Yen T C, Yu J S, Chang K P: Overexpression of BST2 is associated with nodal metastasis and poorer prognosis in oral cavity cancer. Laryngoscope 2014, 124:354-360.
7. Wainwright D A, Balyasnikova I V, Han Y, Lesniak M S: The expression of BST2 in human and experimental mouse brain tumors. Exp Mol Pathol 2011, 91:440-446.
8. Wong Y F, Cheung T H, Lo K W, Yim S F, Siu N S, Chan S C, Ho T W, Wong K W, Yu M Y, Wang V W, Li C, Gardner G J, Bonome T, Johnson W B, Smith D I, Chung T K, Birrer M J: Identification of molecular markers and signaling pathway in endometrial cancer in Hong Kong Chinese women by genome-wide gene expression profiling. Oncogene 2007, 26:1971-1982.
9. Schliemann C, Roesli C, Kamada H, Borgia B, Fugmann T, Klapper W, Neri D: In vivo biotinylation of the vasculature in B-cell lymphoma identifies BST-2 as a target for antibody-based therapy. Blood 2010, 115:736-744.
10. Cai D, Cao J, Li Z, Zheng X, Yao Y, Li W, Yuan Z: Up-regulation of bone marrow stromal protein 2 (BST2) in breast cancer with bone metastasis. BMC Cancer 2009, 9:102.
11. Jones P H, Mahauad-Fernandez W D, Madison M N, Okeoma C M: BST-2/tetherin is overexpressed in mammary gland and tumor tissues in MMTV-induced mammary cancer. Virology 2013, 444:124-139.
12. Matsuda A, Suzuki Y, Honda G, Muramatsu S, Matsuzaki O, Nagano Y, Doi T, Shimotohno K, Harada T, Nishida E, Hayashi H, Sugano S: Large-scale identification and characterization of human genes that activate NF-kappaB and MAPK signaling pathways. Oncogene 2003, 22:3307-3318.
13. Galao R P, Le Tortorec A, Pickering S, Kueck T, Neil S J: Innate sensing of HIV-1 assembly by Tetherin induces NFkappaB-dependent proinflammatory responses. Cell Host Microbe 2012, 12:633-644.
14. Tokarev A, Suarez M, Kwan W, Fitzpatrick K, Singh R, Guatelli J: Stimulation of NF-kappaB activity by the HIV restriction factor BST2. J Virol 2013, 87:2046-2057.
15. Neil S J, Zang T, Bieniasz P D: Tetherin inhibits retrovirus release and is antagonized by HIV-1 Vpu. Nature 2008, 451:425-430.
16. Jones P H, Maric M, Madison M N, Maury W, Roller R J, Okeoma C M: BST-2/tetherin mediated restriction of chikungunya (CHIKV) VLP budding is counteracted by CHIKV non-structural protein 1 (nsP1). Virology 2013, 438:37-49.
17. Jones P H, Mehta H V, Maric M, Roller R J, Okeoma C M: Bone marrow stromal cell antigen 2 (BST-2) restricts mouse mammary tumor virus (MMTV) replication in vivo. Retrovirology 2012, 9:10.
18. Yoo H, Park S H, Ye S K, Kim M: IFN-gamma-induced BST2 mediates monocyte adhesion to human endothelial cells. Cell Immunol 2011, 267:23-29.
19. Ozaki S, Kosaka M, Wakahara Y, Ozaki Y, Tsuchiya M, Koishihara Y, Goto T, Matsumoto T: Humanized anti-HM1.24 antibody mediates myeloma cell cytotoxicity that is enhanced by cytokine stimulation of effector cells. Blood 1999, 93:3922-3930.
20. Yi E H, Yoo H, Noh K H, Han S, Lee H, Lee J K, Won C, Kim B H, Kim M H, Cho C H, Ye S K: BST-2 is a potential activator of invasion and migration in tamoxifen-resistant breast cancer cells. Biochem Biophys Res Commun 2013, 435:685-690.
21. Sayeed A, Luciani-Torres G, Meng Z, Bennington J L, Moore D H, Dairkee S H: Aberrant regulation of the BST2 (Tetherin) promoter enhances cell proliferation and apoptosis evasion in high grade breast cancer cells. PLoS One 2013, 8:e67191.
22. Faustino-Rocha A, Oliveira P A, Pinho-Oliveira J, Teixeira-Guedes C, Soares-Maia R, da Costa R G, Colaco B, Pires M J, Colaco J, Ferreira R, Ginja M: Estimation of rat mammary tumor volume using caliper and ultrasonography measurements. Lab Anim 2013, 42:217-224.
23. Qiu H, Kuolee R, Harris G, Chen W: Role of NADPH phagocyte oxidase in host defense against acute respiratory Acinetobacter baumannii infection in mice. Infect Immun 2009, 77:1015-1021.
24. Jones P H, Okeoma C M: Phosphatidylinositol 3-kinase is involved in Toll-like receptor 4-mediated BST-2/Tetherin regulation. Cell Signal 2013, 25:2752-2761.
25. Ivshina A V, George J, Senko O, Mow B, Putti T C, Smeds J, Lindahl T, Pawitan Y, Hall P, Nordgren H, Wong J E, Liu E T, Bergh J, Kuznetsov V A, Miller L D: Genetic reclassification of histologic grade delineates new clinical subtypes of breast cancer. Cancer Res 2006, 66:10292-10301.
26. Kretschmer C, Sterner-Kock A, Siedentopf F, Schoenegg W, Schlag P M, Kemmner W: Identification of early molecular markers for breast cancer. Mol Cancer 2011, 10:15.
27. Casey T, Bond J, Tighe S, Hunter T, Lintault L, Patel O, Eneman J, Crocker A, White J, Tessitore J, Stanley M, Harlow S, Weaver D, Muss H, Plaut K: Molecular signatures suggest a major role for stromal cells in development of invasive breast cancer. Breast Cancer Res Treat 2009, 114:47-62.
28. University of Iowa Institute for Clinical and Translational Science (ICTS) Compass. [https://research.icts.uiowa.edu/compass]
29. Ewens A, Mihich E, Ehrke M J: Distant metastasis from subcutaneously grown E0771 medullary breast adenocarcinoma. Anticancer Res 2005, 25:3905-3915.
30. Heppner G H, Miller F R, Shekhar P M: Nontransgenic models of breast cancer.

Breast Cancer Res 2000, 2:331-334.
31. Tao K, Fang M, Alroy J, Sahagian G G: Imagable 4T1 model for the study of late stage breast cancer. BMC Cancer 2008, 8:228.
32. Tiffen J C, Bailey C G, Ng C, Rasko J E, Holst J: Luciferase expression and bioluminescence does not affect tumor cell growth in vitro or in vivo. Mol Cancer 2010, 9:299.
33. Kalluri R, Zeisberg M: Fibroblasts in cancer. Nat Rev Cancer 2006, 6:392-401.
34. Ruoslahti E: Fibronectin in cell adhesion and invasion. Cancer Metastasis Rev 1984, 3:43-51.
35. Kim Y N, Koo K H, Sung J Y, Yun U J, Kim H: Anoikis resistance: an essential prerequisite for tumor metastasis. Int J Cell Biol 2012, 2012:306879.
36. Zhu T, Starling-Emerald B, Zhang X, Lee K O, Gluckman P D, Mertani H C, Lobie P E: Oncogenic transformation of human mammary epithelial cells by autocrine human growth hormone. Cancer Res 2005, 65:317-324.
37. Feng M, Li Z, Aau M, Wong C H, Yang X, Yu Q: Myc/miR-378/TOB2/cyclin D1 functional module regulates oncogenic transformation. Oncogene 2011, 30:2242-2251.
38. Coghlin C, Murray G I: Current and emerging concepts in tumour metastasis. J Pathol 2010, 222:1-15.
39. Mareel M, Leroy A: Clinical, cellular, and molecular aspects of cancer invasion. Physiol Rev 2003, 83:337-376.
40. Steeg P S: Tumor metastasis: Mechanistic insights and clinical challenges. Nat Med 2006, 12:895-904.
41. Mahauad-Fernandez W D, Jones P H, Okeoma C M: Critical role for BST-2 in acute Chikungunya virus infection. J Gen Virol 2014, 95:2450-2461.
42. El Fatemi H, Chahbouni S, Jayi S, Moumna K, Melhouf M A, Bannani A, Mesbahi O, Amarti A: Luminal B tumors are the most frequent molecular subtype in breast cancer of North African women: an immunohistochemical profile study from Morocco. Diagn Pathol 2012, 7:170.
43. Fawzi ASE: The prognostic significance of the luminal A, luminal B, basal and Her 2 neu subtypes of breast cancer in Saudi women. Open Breast Cancer J 2013, 5:16-22.
44. Woopen H, Sehouli J: Current and future options in the treatment of malignant ascites in ovarian cancer. Anticancer Res 2009, 29:3353-3359.
45. Ayantunde A A, Parsons S L: Pattern and prognostic factors in patients with malignant ascites: a retrospective study. Ann Oncol 2007, 18:945-949.
46. Frampton J E: Catumaxomab: in malignant ascites. Drugs 2012, 72:1399-1410.
47. Mackey M F, Barth R J Jr, Noelle R J: The role of CD40/CD154 interactions in the priming, differentiation, and effector function of helper and cytotoxic T cells. J Leukoc Biol 1998, 63:418-428.
48. Petrelli F, Borgonovo K, Lonati V, Elia S, Barni S: Regression of liver metastases after treatment with intraperitoneal catumaxomab for malignant ascites due to breast cancer. Target Oncol 2013, 8:291-294.
49. Buckman R, De Angelis C, Shaw P, Covens A, Osborne R, Kerr I, Reed R, Michaels H, Woo M, Reilly R, Law J, Baumal R, Groves E, Marks A: Intraperitoneal therapy of malignant ascites associated with carcinoma of ovary and breast using radioiodinated monoclonal antibody 2G3. Gynecol Oncol 1992, 47:102-109.
50. Dolcetti L, Marigo I, Mantelli B, Peranzoni E, Zanovello P, Bronte V: Myeloid-derived suppressor cell role in tumor-related inflammation. Cancer Lett 2008, 267:216-225.
51. Talmadge J E: Pathways mediating the expansion and immunosuppressive activity of myeloid-derived suppressor cells and their relevance to cancer therapy. Clin Cancer Res 2007, 13:5243-5248.

### Example 2

Reference is made to the manuscript entitled “Bone Marrow Stromal Antigen 2 (BST-2) DNA is Demethylated in Breast Tumors and Breast Cancer Cells,” Wadie D. Mahauad-Fernandez, Nicholas C. Borcherding, Weizhou Zhang, and Chioma M. Okeoma, PLoS ONE 10(4):e0123931. doi:10.1371/journal.pone.0123931, published on Apr. 10, 2015, the content of which is incorporated herein by reference in its entirety.

Title: Bone Marrow Stromal Antigen 2 (BST-2) DNA is Demethylated in Breast Tumors and Breast Cancer Cells

Abstract

### Background

Bone marrow stromal antigen 2 (BST-2) is a known anti-viral gene that has been recently identified to be overexpressed in many cancers, including breast cancer. BST-2 is critical for the invasiveness of breast cancer cells and the formation of metastasis in vivo. Although the regulation of BST-2 in immune cells is unraveling, it is unknown how BST-2 expression is regulated in breast cancer. We hypothesized that meta-analyses of BST-2 gene expression and BST-2 DNA methylation profiles would illuminate mechanisms regulating elevated BST-2 expression in breast tumor tissues and cells.

Materials and Methods:

We performed comprehensive meta-analyses of BST-2 gene expression and BST-2 DNA methylation in The Cancer Genome Atlas (TCGA) and various Gene Expression Omnibus (GEO) datasets. BST-2 expression levels and BST-2 DNA methylation status at specific CpG sites on the BST-2 gene were compared for various breast tumor molecular subtypes and breast cancer cell lines.

Results:

We show that BST-2 gene expression is inversely associated with the methylation status at specific CpG sites in primary breast cancer specimens and breast cancer cell lines. BST-2 demethylation is significantly more prevalent in primary tumors and cancer cells than in normal breast tissues or normal mammary epithelial cells. Demethylation of the BST-2 gene significantly correlates with its mRNA expression. These studies provide the initial evidence that significant differences exist in BST-2 DNA methylation patterns between breast tumors and normal breast tissues, and that BST-2 expression patterns in tumors and cancer cells correlate with hypomethylated BST-2 DNA.

### Conclusion

Our study suggests that the DNA methylation pattern and expression of BST-2 may play a role in disease pathogenesis and could serve as a biomarker for the diagnosis of breast cancer.

### Introduction

Breast cancer is the second largest cause of cancer-related deaths in women according to the National Cancer Institute (NCI) and is the second most common cancer diagnosed in women. Treatment for breast cancer is dependent on its subtype classification [1]. The most severe forms of breast cancer which respond poorly to hormonal or targeted therapies include luminal B and basal breast cancers [2,3]. The inability to develop new treatments is partially due to a limited understanding of all the drivers of these malignancies which give transformed cells a selective advantage over normal cells to grow and thrive in unfavorable environments.

One of the drivers of breast malignancy is BST-2 [4], also known as Tetherin, CD317, or HM1.24. BST-2 is an IFN-inducible type II transmembrane protein expressed mainly at the surface of cells [5,6]. BST-2 contains an N-terminus cytoplasmic tail followed by a transmembrane domain, an extracellular coiled-coiled domain and a C-terminus glycophosphatidylinositol (GPI) anchor embedded in lipid rafts along the cell membrane [5,7]. BST-2 was discovered as a marker of differentiated B cells [8] and was later rediscovered as a potent antiviral restriction factor with the ability to tether enveloped viruses to the cell membrane of infected cells via its GPI anchor [9-12], as well as to potently inhibit virus replication in cultured cells and in vivo [11, 13, 14]. BST-2 is thought to mediate host immune response by activating NF-κB through interaction with transforming growth factor beta-activated kinase 1(TAK1) and TNF receptor associated factors (TRAFs) 2 and 6 [14-16]. In addition, BST-2 induces antibody-dependent cell cytotoxicity (ADCC) against the envelope protein of HIV [17-19].

Recent studies have demonstrated that the mRNA and protein expression of BST-2 are elevated in various cancers including: head and neck cancer, oral cavity cancer, glioblastoma, lung cancer, endometrial cancer, lymphomas, and breast cancers [20-26]. There is direct evidence for a role for BST-2 in two cancers. BST-2 antibody-mediated ADCC has been shown to be potent in myeloma treatment [27,28] and in breast cancer, BST-2 plays a direct role in driving breast malignancy [4]. In vivo, elevated BST-2 expression is associated with primary tumor growth, metastasis, and poor prognosis [4]. Upon BST-2 knockdown, breast cancer cells lose their capacity to grow and thrive in vivo [4]. The molecular mechanisms involved in BST-2-mediated breast cancer malignancy includes; BST-2-meditated enhancement in cancer cell i) adhesion to the tumor microenvironment, ii) migration through the basement membrane, iii) invasion through extracellular matrix lattice, and anchorage independent growth [4,29]. In contrast to breast cancer, knock down of BST-2 in glioblastoma had no effect on tumor growth in mice [22].

Despite the functions of BST-2 in breast oncogenesis, little is known about the regulation of BST-2 expression in cancer cells. Sayeed et al., (2013) reported that BST-2 expression in tumor tissues and primary breast cancer cell lines is negatively regulated by transforming growth factor beta (TGF-β) [30]. However, there is no evidence for genetic or epigenetic modifications that regulate BST-2 expression in breast cancer tissue/cells.

The process of carcinogenesis is characterized by genetic and epigenetic modifications. Epigenetic alterations and regulation of gene functions is increasingly being recognized as critical in carcinogenesis [31]. These alterations may involve histone modifications and changes in DNA methylation status of cytosine bases (C) in the context of CpG dinucleotides.

The result of alterations in DNA methylation status is changes in gene expression patterns that may perturb normal cell physiology and function. There is an inverse correlation between gene expression and DNA methylation status [32,33]. As such, hypermethylation of DNA silences gene expression [32] whereas hypomethylation or demethylation of DNA enables gene expression [34]. Both hypermethylation and hypomethylation play important but distinct roles in the initiation, progression, and metastasis of various cancers [35,36]. Here, we aimed to determine the source of BST-2 overexpression in breast tumors through in silico and in vitro analyses. We report that BST-2 expression in breast tumors and cancer cells is epigenetically regulated by hypomethylation or demethylation of specific CpG sites along the BST-2 gene.

Methods.

Cell Lines:

Normal human mammary epithelial cell line HMLE, luminal A breast cancer cell lines MCF-7 and T47D, luminal B cell line BT-474, HER2+ cell line SK-BR-3, triple negative MDA-MB-231 cell line, and basal breast cancer cell line SUM-159 are from ATCC and were maintained according to ATCC instructions.

Gene Expression and Methylation Analysis:

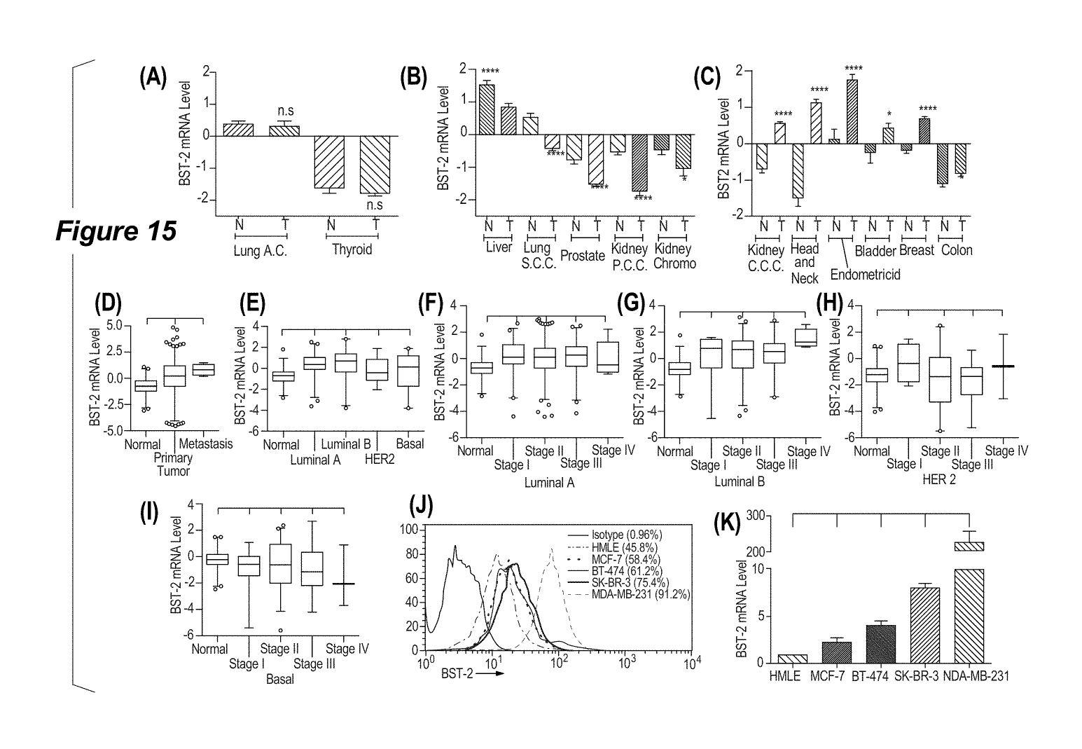

The UCSC Cancer Genome Browser (https://genome-cancer.ucsc.edu) [37] was used to assess BST-2 expression for the PAN-CAN-normalized samples [38,39] for the indicated cancer types and their corresponding normal tissues. Separately, expression and methylation values from the individual BRCA cohort of the TCGA were used. Expression versus methylation analyses were performed with mean-centralized level 3 Illumina HiSeq 2000 RNAseq expression data and Infinium HumanMethylation450 beta-values. Methylation beta-values are reported as either an average of all probes or by the specific probe for BST-2. Probes on the BST-2 gene (Chromosome 19) used in these analyses are: probe 1 cg22282590 (position: 17514117), probe 2 cg07839313 (position: 17514600), probe 3 cg12090003 (position: 17516282), probe 4 cg16363586 (position: 17516329), probe 5 cg11558551 (position: 17516442), probe 6 cg01254505 (position: 17516470), probe 7 cg01329005 (position: 17516712), probe 8 cg09993699 (position: 17517008) and probe 9 cg20092122 (position: 17517221). Probe sequences can be downloaded at Illumina Infinium HumanMethylation450K Bead Chip product page at http://support.illumina.com/array/array_kits/infinium_humanmethylation450_beadchip_kit/down loads.html. Samples were divided into indicated categorical groups using the Biotab clinical information available at the TCGA DCC (https://tcga-data.nci.nih.gov/tcga/). Differences in sample number in figures are a result of sorting by categorical data, i.e. primary tumor samples that have PAM50 subtypes are less than the total number of primary samples with RNAseq expression. Expression values were also sorted by sample type, PAM50 subtype from RNAseq (TCGA AWG), and pathological stage. Analysis of BST-2 expression from different breast cancer subtypes was performed only with normal and primary tumor samples. Data from metastatic tumors were excluded from those analyses (<10 metastatic samples), but were used for the analysis of BST-2 expression between normal mammary tissue (Normal), primary tumors (Tumor), and metastatic tumors (Metastatic) (FIG. 15D). All TCGA data was processed and analyzed using Graph Prism software. In addition, five datasets from the Gene Expression Omnibus (http://www.ncbi.nlm.nih.gov/geo/) were used: 1) GEO dataset GSE10797 [40] was used to analyze BST-2 and APOBEC3G (A3G) expression in the epithelium and stroma from normal and cancer tissues of patients undergoing surgical resection or reduction mammoplasty, 2) expression of BST-2 in different human breast cancer cell lines was analyzed using the dataset GSE41313 (only data for MCF-7, T47D, MDA-MB-231, and SUM-159 cells were downloaded) [41], 3) methylation and 4) expression pattern of the BST-2 gene in different human breast cancer cell lines was analyzed with GEO datasets GSE49794 and GSE45732, respectively. These datasets encompass a data superseries derived from the same cells and the same experiment [42], and 5) the effects of 5-aza-2′-deoxycytidine (DAC) treatment on BST-2, Claudin-6, and GAPDH expression in several human breast cancer cell lines was evaluated with the dataset GSE28976 (for HMLE, SK-BR-3 and MDA-MB-231 cells only) [43] and GSE36683 [44] (for MCF-7 cells only). Control and DAC treated samples were used. Cell line methylation versus expression analysis was conducted using the same methylation probe IDs listed previously. Methylation probes are reported as either independent beta-values or the average of probes 3-9 beta-values where indicated. Expression was derived from RNAseq data of GSE45732 and reported in reads per kilobases per million reads mapped (RPKM). All GEO data sets were processed and analyzed using Graph Prism software.

5-Azacytidine Treatment and Flow Cytometry:

Cell lines of interest were plated at 150,000 cells/well in a 6-well plate and treated with DMSO (vehicle) or 1 μM of 5-azacytidine (5-azaC, Sigma Aldrich) for 5 days. Cells were harvested using 0.25% trypsin-EDTA (Mediatech, Corning, N.Y., USA). Post-incubation, 8 ml of 10% FBS-containing RPMI media (Life Technologies) was added and cells were centrifuged. Media was aspirated and individual cells were resuspended in 2% FBS-containing PBS (Life Technologies). Cell suspension was passed through a 40 μM cell strainer (Falcon). Cells were incubated at 4° C. for 1 hour with APC-conjugated anti-BST-2 primary antibodies or appropriate IgG (Ebioscience) and washed with 1×PBS. After washing, cells were incubated with the fluorescent intercalator-7-aminoactinomycin D (7-AAD) (BioLegend) at 4° C. for 30 minutes to exclude dead cells. Stained cells were quantified using a FACS Calibur flow cytometer (BD). 10,000 events were collected per sample and FACS data were analyzed and plotted using Flowjo software (TreeStar).

5-Azacytidine Treatment and Reverse Transcriptase Quantitative Real Time PCR (RT-qPCR):

Human normal and breast cancer cell lines were plated at 150,000 cells/well in a 6-well plate and treated with DMSO (vehicle) or 1 μM of 5-azacytidine (5-azaC, Sigma Aldrich) for 5 days. Cells were lifted using Versene (a gentle cell dissociator, Life Technologies), washed with PBS, pelleted and stored at −20° C. until required for analysis. RNA was isolated from frozen cells using the RNeasy mini kit (Qiagen) according to manufacturer's instructions. Equivalent amounts of RNA were treated with DNase (Qiagen). A portion of RNA was subjected to cDNA synthesis (ABI) as previously described [12, 45-47]. RNA concentration and purity were assessed at 260/280 nm using the spectrophotometer. Using synthesized cDNA, sequence-specific primers were used to amplify BST-2 [4] and GAPDH [13,48]. Claudin-6 was amplified with (F: GGAGGAGAAGGATTCCAAGG, R: AGCCACCAGGGGGTTATAGA) primer pair. RT-qPCR was carried out with an ABI 7500 FAST thermal cycler in triplicates as previously described [11-13, 45, 46, 48-50].

Statistical Analysis:

Statistical analysis of significant differences was performed with unpaired t test with Welch's correction (GraphPad Prism software). Error bars represent standard error of the mean (SEM) or 95% confidence interval (CI) of the mean. Correlation studies were carried out using GraphPad Prism software to calculate r² and p values. r² values of −0.30 or lower (inverse correlations) were considered significant. A p value of 0.05 or lower was considered significant.

Results

BST-2 is Differentially Expressed in Various Cancers.

To understand the spectrum of BST-2 expression in various cancers, we analyzed the expression pattern of BST-2 mRNA in various tumors across the TCGA. Results reveal that levels of BST-2 expression in various cancer types differ. Compared to normal tissues, BST-2 expression in tumors is either unchanged (FIG. 15A), significantly downregulated (FIG. 15B), or significantly elevated (FIG. 15C). In breast cancer, BST-2 expression is highest in metastatic tumors compared to primary tumors, and normal mammary gland tissues have the lowest expression (FIG. 15D). Evaluation of BST-2 expression in different breast cancer subtypes show that while all cancer subtypes have elevated BST-2, tumors categorized as luminal B subtype has the highest BST-2 level compared to normal mammary tissue (FIG. 15E). Although BST-2 expression in HER2 and basal tumor subtypes is not statistically different from normal tissues, it is well stablished that BST-2 is overexpressed in the most aggressive forms of breast cancer [4,30]. The lack of significance could be due to the high variability within the basal group (FIG. 15E) and fewer data points for HER2 subtype. In addition, data points used in our analysis came from primary tumors only because metastatic samples within each subtype were excluded since there were insufficient data points for metastatic tumors.

Comparison of BST-2 expression levels in the different stages within the different subtypes show significant disease stage-dependent differences in BST-2 mRNA in the luminal tumor types compared to normal breast tissue (FIGS. 15F and 15I). However, BST-2 expression in the different disease stages of HER2 (FIG. 15H) and basal (FIG. 15I) tumor types was not different from normal tissues except for a modest significant difference (p<0.0313) in stage II of basal tumors (FIG. 15I). Host dependent variability in BST-2 levels and/or the small number of data points may have affected statistical analysis. Data presented in FIGS. 15E to 15I do not include metastatic tumors because there were few data points available.

To evaluate the level of BST-2 expression in different metastatic cells, we utilized breast cancer cell lines that originated from various metastatic sites. BST-2 expression both at the RNA (FIG. 15J) and protein (FIG. 15K) in different metastatic cells directly correlates to their aggressiveness. As such, triple-negative MDA-MB-231 cells presented the highest BST-2 levels followed by HER2 metastatic SK-BR-3 cells, while the luminal cell lines (MCF-7 and BT-474) express lower BST-2 levels (FIGS. 15J and 15K). These data support the premise that BST-2 levels are elevated in primary and metastatic breast tumor tissues and cell lines.

BST-2 is Hypomethylated in Breast Tumors.

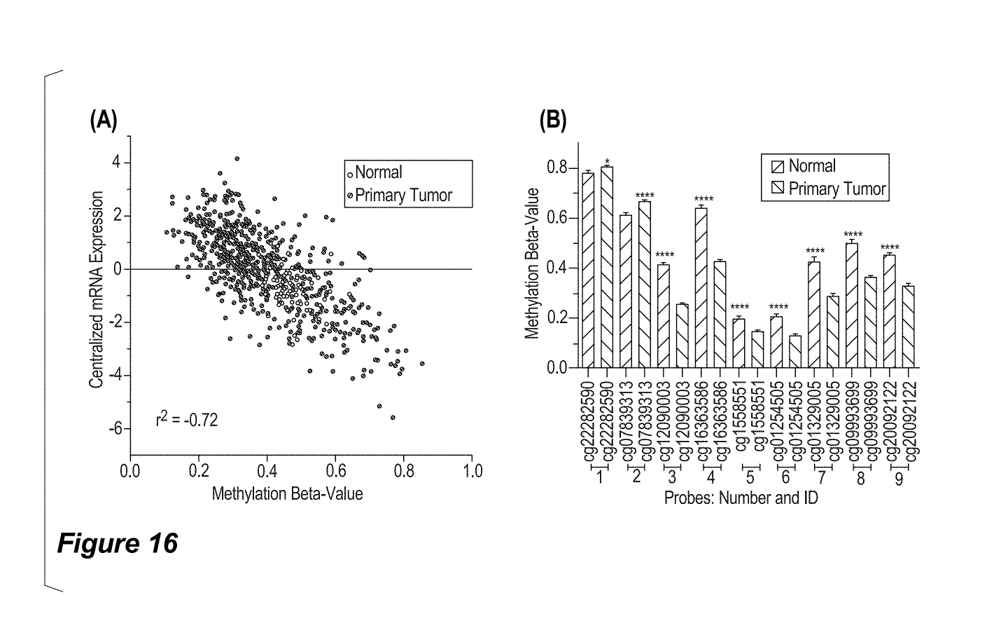

To probe into the regulatory mechanism of BST-2 overexpression in breast tumors, we analyzed BST-2 methylation beta-values from paired tumor and normal breast tissues. The methylation beta-value was plotted against the corresponding RNA expression. Results reveal that BST-2 mRNA expression is inversely correlated to its DNA methylation status (FIG. 16A) with a highly significant r² value of −0.720. The inverse correlation of BST-2 mRNA with BST-2 DNA methylation beta-value signifies that in tumors, BST-2 is hypo- or demethylated. This prompted us to query the differences in BST-2 methylation across the entire gene. Comparing paired tumor and normal mammary tissues from the TCGA repository, we found that 7 CpG sites represented by probes 3 to 9 (see methods section for probe IDs) were significantly hypomethylated in tumors compared to normal tissues (FIG. 16B). In contrast to probes 3 to 9, methylation beta-values for probes 1 and 2 show that these CpG sites are hypermethylated in breast tumors. Overall, these data show that BST-2 DNA is hypomethylated in mammary tumors compared to normal mammary tissue.

Hypomethylation of Specific CpG Sites Correlate with BST-2 Expression in Tumors.

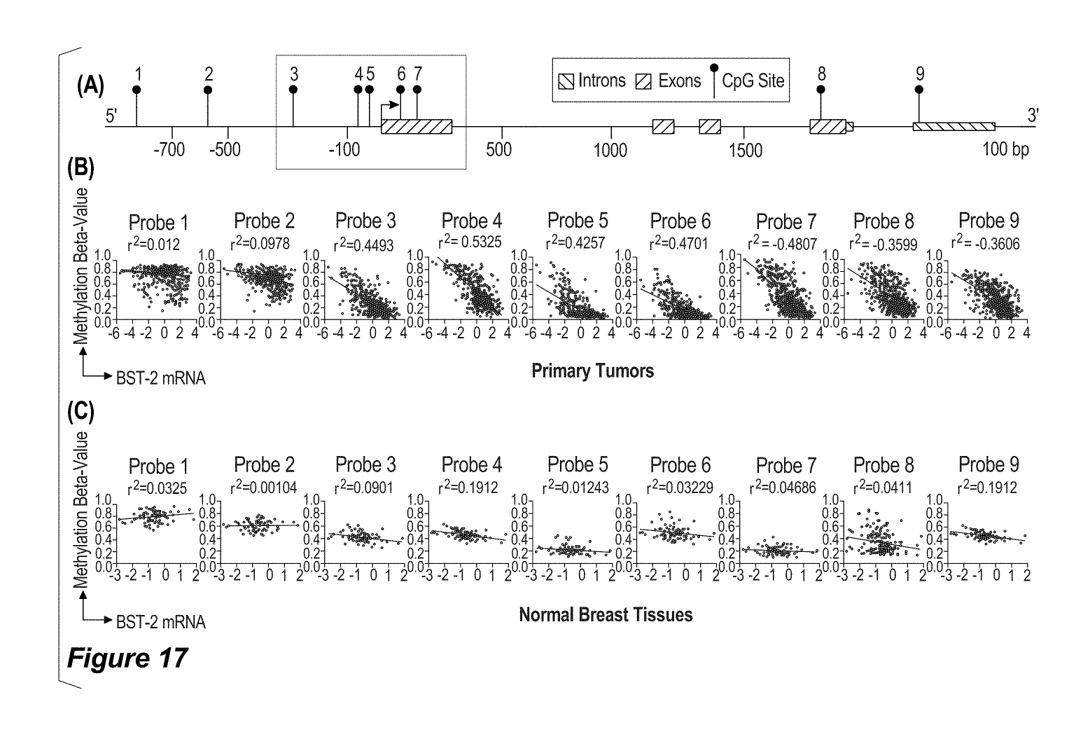

To better understand the effect of BST-2 methylation on BST-2 expression, we sought to identify CpG sites in the BST-2 gene (FIG. 17A), wherein methylation status was associated with elevated BST-2 expression in tumors compared to normal breast tissues. FIG. 17A portrays the location of the different probes used in the Human Methylation 450 array across the BST-2 gene as described in the methods section. CpG sites corresponding to probes 3 to 7 which are proximal to the BST-2 gene transcription start site (TSS) had the lowest R-squared (r², ranging from −0.4257 to −0.5325) values when plotted against corresponding BST-2 expression in primary tumors (FIG. 17B). In addition, CpG sites downstream (probes 8 and 9) of the TSS did show a significant inverse correlation with tumor tissue BST-2 mRNA expression based on r² values of −0.3599 to −0.3606 (FIG. 17B). Moreover, CpG sites upstream (probes 1 and 2) of the TSS are hypermethylated (FIGS. 16A and 16B) and did not show a strong correlation with tumor tissue BST-2 mRNA expression based on r² values of −0.0978 to 0.012 (FIG. 17B). In parallel, correlation between BST-2 methylation beta-values and BST-2 mRNA in normal breast tissues was performed. There was no inverse correlation between CpG methylation and BST-2 expression with any of the 9 probes; r² values range from −0.1912 to 0.03254 (FIG. 17C). These data suggest that the CpG sites proximal to BST-2 gene TSS and downstream of the TSS may be responsible for transcriptional regulation of BST-2 in breast tumors.

Hypomethylation of CpG Sites Proximal to the BST-2 Promoter Correlate with BST-2 Overexpression in Different Breast Tumor Subtypes.

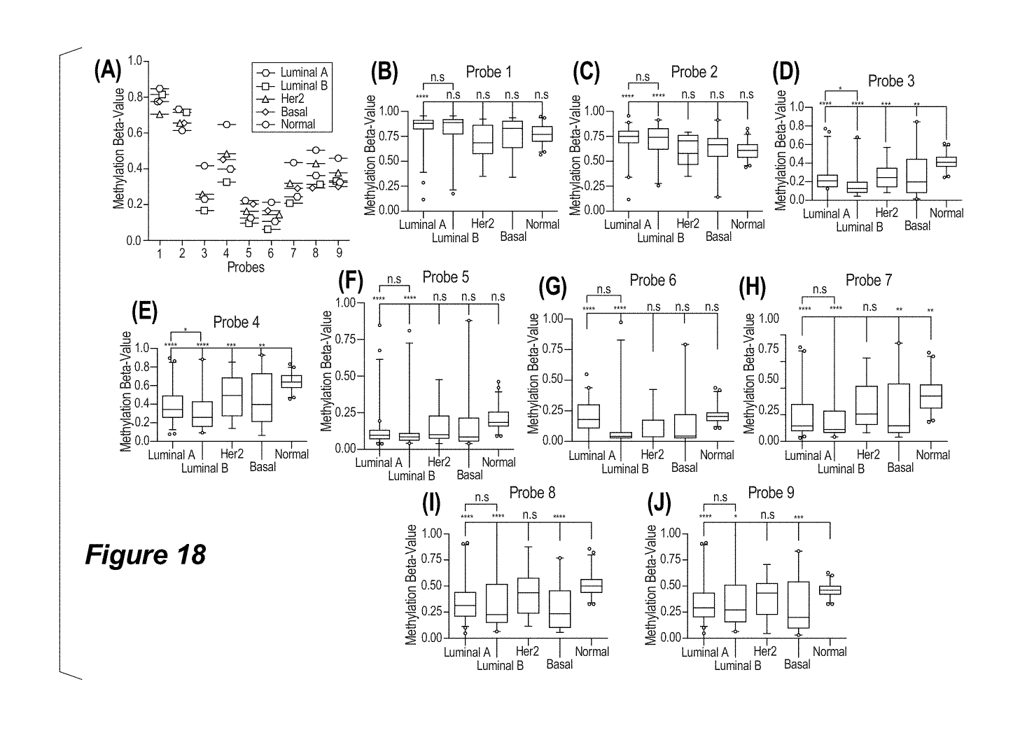

Since demethylation of CpG sites strongly associates with increased expression of BST-2 in tumor tissues, we next analyzed the methylation level of BST-2 in different breast cancer subtypes. We found that BST-2 is hypomethylated on the CpG sites represented by probes 3 to 9 irrespective of tumor subtype (FIG. 18A). To an extent, BST-2 expression could be predicted by the degree of methylation of these probes (FIG. 18A). As such, CpG sites represented by probes 1 and 2 do not mirror BST-2 expression in tumors and were hypermethylated (FIGS. 18A to 18C). For example, in luminal A tumors, CpG sites represented by probes 1 and 2 are hypermethylated compared to normal breast tissues. Luminal B tumors have the highest level of BST-2 mRNA (FIG. 15C), and presents the lowest methylation values among probes 3, 4, 5, 6, and 7 (FIGS. 18A to 18H). Compared to normal breast tissues, the luminal tumor types (A and B) are differentially methylated across all probes compared to normal tissues (FIGS. 18B to 18J). Probes 3 to 9 are hypomethylated in luminal tumors compared to normal tissues while probes 1 and 2 in these tumors are hypermethylated compared to normal tissues, except for probe 1 in luminal B tumors that has a non-significant methylation beta-value compared to normal tissues (FIG. 18B). The HER2 tumor type is hypomethylated at CpG sites represented by probes 3 and 4 (FIGS. 18D and 18E); while the basal tumor subtype is hypomethylated at CpG sites corresponding to probes 3, 4, 7, 8, and 9 (FIGS. 18D, 18E, 18H to 18J). These data suggest that BST-2 methylation pattern at different CpG sites could predict breast cancer subtype (Table 1).

BST-2 mRNA Expression Correlates with DNA Hypomethylation in Breast Cancer Epithelial Cells.

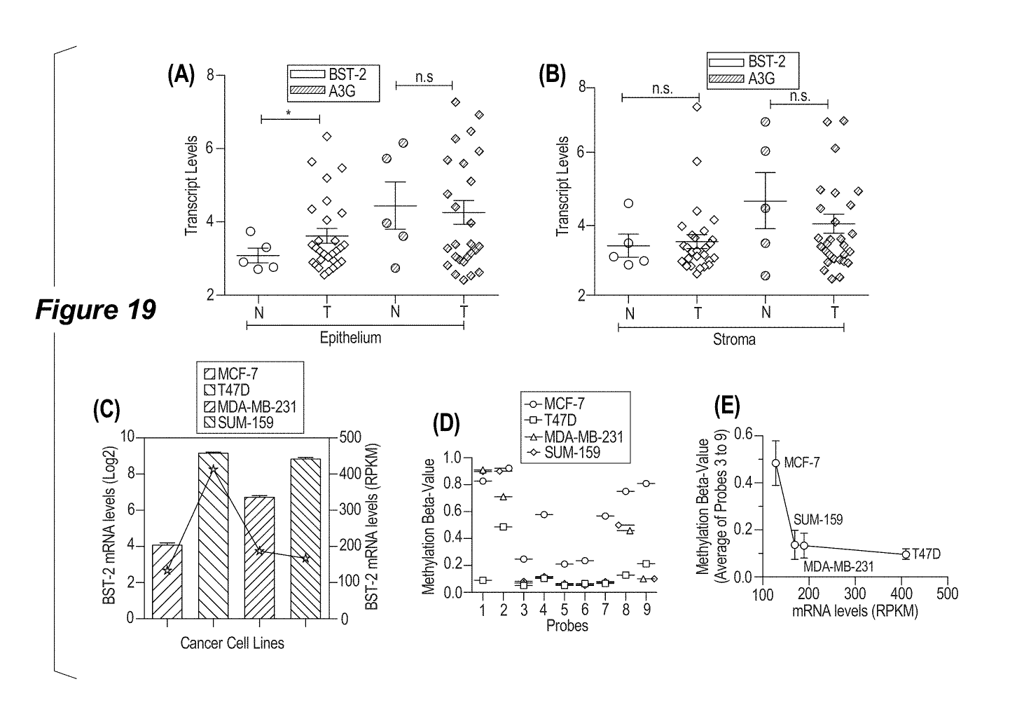

In breast carcinomas, neoplastic epithelial cells coexist and interact with various stromal cells that together create the tumor microenvironment. While neoplastic epithelial cells have higher BST-2 mRNA compared to normal cells, there was no difference in the expression pattern of another antiviral gene called Apobec3G (A3G) in these epithelial cells (FIG. 19A). This data support the preposition that neoplastic epithelial cells are the source of elevated BST-2 expression in tumors [4]. Analysis of the stromal cells for BST-2 and A3G expression confirmed that cancer epithelial cells are the source of elevated BST-2 in tumors (FIG. 19B). On the basis of this finding, we assessed correlation in patterns of BST-2 mRNA expression and BST-2 methylation in well-established breast cancer cell lines downloaded from GEO dataset GSE10797 [31]. Our analysis revealed that elevated, levels of BST-2 mRNA varied among different breast cancer cell lines; with MCF-7 cells expressing the least BST-2 mRNA (FIG. 19C, bar graph (GSE10797) and line graph (GSE49794)). Importantly, the relative levels of BST-2 expression are similar between the two datasets used (FIG. 19C, bar and line graphs). As expected, level of BST-2 expression was inversely correlated to the methylation status of CpG sites represented by probes 3 to 9 (FIG. 19C line graph, and 5D). As such, MCF-7 cells which have the lowest BST-2 mRNA levels (FIG. 19C) have the highest methylation beta-values on probes 3 to 9 than any of the other cell lines (FIGS. 19D and 19E). Remarkably, high BST-2-expressing luminal A T47D cells, triple-negative MDA-MB-231 cells and basal SUM-159 cells are significantly hypomethylated (methylation beta-values of 0.21127, 0.10207 and 0.10295, respectively) on CpG sites corresponding to probe 9 (FIG. 19D).

Moreover, the average methylation beta-value for probes 3 to 9 was inversely correlated to their corresponding BST-2 mRNA levels among all breast cancer cell lines analyzed (FIG. 19E), providing additional support for a link between BST-2 expression and methylation status. Together with tumor data presented in FIG. 18 and Table 1, it appears that BST-2 expression is increased by hypomethylation of CpG sites adjacent and downstream of the BST-2 TSS. Furthermore, in aggressive breast cancers and cancer cell lines, such as basal and triple-negative tumors, BST-2 is hypomethylated at CpG sites downstream of the BST-2 TSS.

Cancer Cells with Elevated BST-2 Levels are Unresponsive to 5-Azacytidine Induced BST-2 DNA Demethylation.

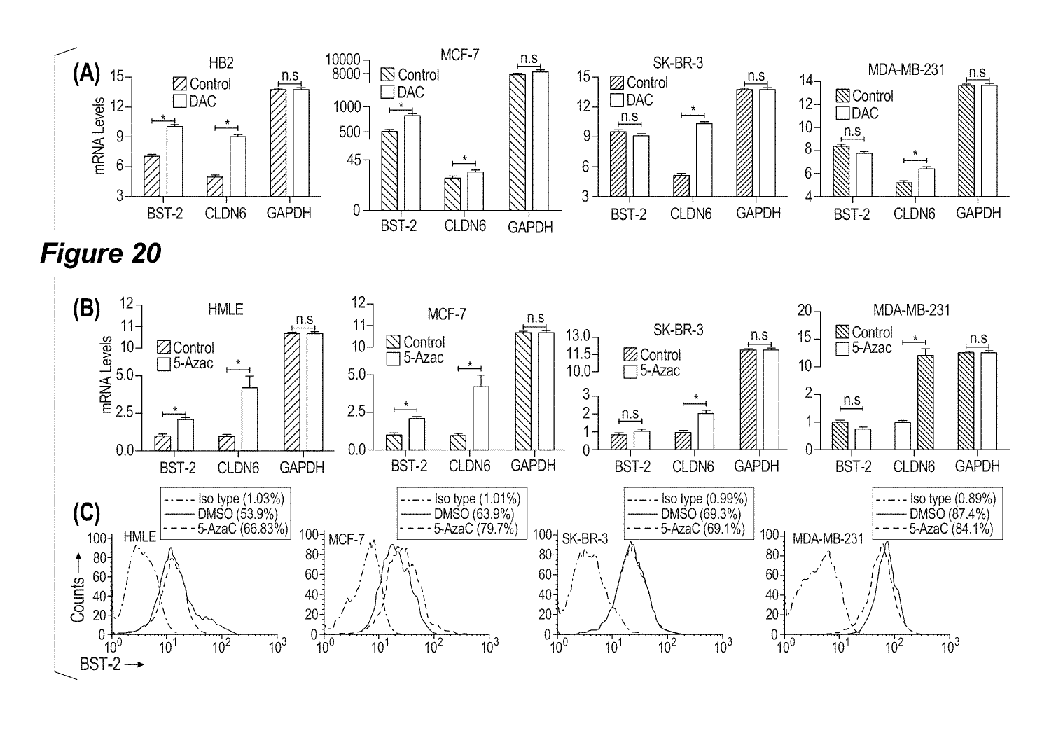

2′-deoxy-5-azacytidine (decitabine, DAC) is a deoxycytidine which incorporates into replicating DNA and prevents DNA methylation, thus, resulting in DNA hypomethylation and upregulation of gene expression. Since levels of BST-2 expression varies among cancer cells, we predicted that treatment with demethylating agents will further elevate BST-2 expression in cancer cells such as MCF-7 that express low BST-2, but that such treatment will have no effect on high BST-2 expressing cells. For this purpose, we used GEO datasets GSE28976 [43] and GSE36683 [44] (for MCF-7s only) to analyze the effect of DAC on BST-2 methylation in several human breast cancer cell lines. We found that DAC treatment of normal breast cell line HB2 and low BST-2-expressing luminal A cell line MCF-7 led to increased BST-2 mRNA expression (FIG. 20A). However, DAC-treated high BST-2-expressing HER2 SK-BR-3 and triple-negative MDA-MB-231 cells did not show any increase in BST-2 levels (FIG. 20A). Importantly, mRNA levels of the tumor suppressor gene claudin-6 (CLDN6) reported to be increased in breast cancer cells upon DAC treatment [51] were induced in all cell types regardless of their subtype classification, while GAPDH mRNA, a house keeping gene did not change following DAC treatment (FIG. 20A).

GEO data were validated by treating cells with the nucleoside analogue 5-azacytidine (azacytidine, 5-AZaC) and analyzing BST-2, CLDN6 and GAPDH mRNA levels (FIG. 20B) and BST-2 protein levels (FIG. 20C). In agreement with the GEO datasets in FIG. 6A, HMLE (normal breast epithelial cell line) and MCF-7 cells treated with 5-AzaC showed a significant increase in BST-2 levels both at the RNA and protein levels (FIGS. 20B and 20C), while levels of BST-2 mRNA and protein following 5-AzaC treatment were unchanged in high BST-2-expressing HER2 SK-BR-3 and triple negative MDA-MB-231 cells (FIGS. 20B and 20C). As expected, CLDN6 was induced in all cell types upon 5-AzaC treatment (FIG. 20B), but GAPDH did not change (FIG. 20B). These results suggest that DAC/5-AzaC does not impact BST-2 expression in cancer cells with elevated BST-2 [52,53].

### Discussion

DNA demethylation was the first described epigenetic modification observed in various human cancers compared to normal tissues [54]. Cancer-linked DNA demethylation is associated with metastases of primary tumors [55,56] and is as prevalent as cancer-associated DNA hypermethylation. In cancer genomes, DNA hypermethylation is thought to occur in the promoter regions of tumor suppressor genes, which may lead to silencing of these tumor suppressors [57]. In contrast, DNA hypomethylation frequently occurs in DNA repeats, resulting in genomic instability and mutation in cancer genomes [58-60]. It has been suggested that hypomethylation of immunity-related genes, such as BST-2 may promote carcinogenesis. As such, promoter hypomethylation of IL-10 activates its expression and inhibits the generation of immune response against breast cancer [61], while hypomethylation of the immunogenic antigen SPAN-Xb may result in de novo B-cell response in myeloma cells [62]. In this study, we conducted meta-analysis of the methylation status of the BST-2 gene because BST-2 has been associated with development and progression of breast cancer in vivo [4]. The mechanism for the role of BST-2 in the evolution/progression of breast carcinogenesis is still poorly understood. Nevertheless, RNAi-mediated downregulation of BST-2 increases the survival of tumor-bearing mice [4], suggesting therapeutic significance.

Meta-analyses of human epidemiological data revealed that in breast tumors and neoplastic epithelial cells, BST-2 expression is epigenetically regulated by DNA demethylation. There are unique CpG sites corresponding to probes 3 to 9 from the Human Methylation 450 array and proximal to the BST-2 gene TSS that were demethylated across all breast tumor types (FIG. 18A), irrespective of their subtype classification. In luminal B tumors, CpGs represented by probes 3 to 9 are significantly hypomethylated compared to normal tissues (Table 1); this observation is interesting given that this cancer subtype is considered to have a hypermethylated phenotype among breast cancer subtypes [63]. The CpG sites corresponding to probes 8 and 9 that are downstream and distal to BST-2 TSS were of significance to the triple-negative tumor subtype, as its methylation beta-value was lower compared to other subtypes (FIGS. 18A, 18I, 18J, 19D, and 19E). In contrast, the hypermethylated CpG sites represented by probes 1 and 2 that are upstream and distal to BST-2 TSS may have little or no effect on BST-2 regulation (FIG. 18A to 18C).

Remarkably, the demethylated CpG sites represented by probes 3 to 7 are largely located adjacent to transcription factor binding sites in the BST-2 gene, including those of nuclear factor of activated T-cells (NF-AT), interferon regulatory factor (IRF) [12,64], signal transducers and activators of transcription (STAT), and nuclear factor kappa B (NF-κB) [65]. This observation is interesting because DNA methylation controls gene transcription through interference with the ability of transcription factors to bind to DNA [66]. A phenomenon that could partially explain the significant inverse relationship between BST-2 expression and BST-2 DNA methylation observed on probes 3 to 7. In addition, the inability of high BST-2 expressing MDA-MB-231 and SK-BR-3 cells to respond to DAC- or 5-AzaC-mediated induction of BST-2 expression suggests that high BST-2-expressing aggressive breast cancers at some point may have lost methylation-dependent regulation of BST-2 transcription which results in BST-2 overexpression and promotion of breast malignancy [4]. An alternative explanation could be that demethylating agents had a stabilizing effect on BST-2 in these cells, a phenomenon reported previously for MATN4 and CTSL2 unresponsiveness to 5-AzaC [52,53].

Examples of tumor-related overexpressed genes which become promoter hypomethylated during carcinogenesis includes, but not limited to, Sonic Hedgehog [67], P-cadherin [68], and CDH3 [69], as well as MATN4 and CTSL2 [52,53], supporting the data reported here for BST-2. Indeed, BST-2 overexpression due to DNA hypomethylation has been reported for glioblastoma [70] and lupus [71]. Patients with lupus presented with BST-2 hypomethylation on probes 1 to 7 compared to the controls, pointing to a common mechanism of methylation-dependent BST-2 regulation.

However, we cannot rule out other epigenetic-dependent or -independent sources of BST-2 regulation such as gene amplification, histone posttranslational modifications, increased translation of BST-2 or a decrease in the rate of BST-2 degradation or turnover. Further research is warranted to determine whether there are other mechanisms controlling BST-2 overexpression in breast cancer and whether methylation changes regulate BST-2 expression in other cancers including those in which BST-2 levels are unchanged (FIG. 15A) or suppressed (FIG. 15B) in tumor tissues compared to their corresponding normal tissues. Additionally, it is of interest to determine how changes in BST-2 DNA methylation pattern relate to the molecular pathology in breast cancer initiation, progression, and metastasis.

### Conclusions

We conclude that a greater frequency of BST-2 hypomethylation was observed in breast cancer tissues and cells compared to normal breast tissues and cells. Therefore, BST-2 overexpression from DNA hypomethylation could influence breast carcinogenesis and could predict breast cancer prognosis or therapeutic response.

TABLE 1
Profile of BST-2 DNA hypomethylation in breast cancer subtypes as
ranked by significant difference compared to normal breast tissue

| | Probe name/ | | | | |
|---|---|---|---|---|---|
| Probe ID | CpG site | Luminal A | Luminal B | HER2 | Basal-like |
| cg22282590 | Probe 1 | Hyper (****) | unchanged | unchanged | unchanged |
| cg07839313 | Probe 2 | Hyper (****) | Hyper (****) | unchanged | unchanged |
| cg12090003 | Probe 3 | Hypo (****) | Hypo (****) | Hypo (****) | Hypo (****) |
| cg16363586 | Probe 4 | Hypo (****) | Hypo (****) | Hypo(*) | Hypo (****) |
| cg11558551 | Probe 5 | Hypo (****) | Hypo (****) | unchanged | unchanged |
| cg01254505 | Probe 6 | Hypo (****) | Hypo (****) | unchanged | unchanged |
| cg01329005 | Probe 7 | Hypo (****) | Hypo (****) | unchanged | Hypo (**) |
| cg09993699 | Probe 8 | Hypo (****) | Hypo (****) | unchanged | Hypo (****) |
| cg20092122 | Probe 9 | Hypo (****) | Hypo (*) | unchanged | Hypo (***) |

*Is degree of hyper- or hypo-methylation based on statistical significance relative to normalbreast tissues (unpaired t test with Welch's correction). P value (*), where
*is p < 0.02,
**is p < 0.003,
***is p < 0.0005 and
****is p <0.0001.

### REFERENCES

1. Yersal O, Barutca S (2014) Biological subtypes of breast cancer: Prognostic and therapeutic implications. World J Clin Oncol 5: 412-424.
2. Tran B, Bedard P L (2011) Luminal-B breast cancer and novel therapeutic targets. Breast Cancer Res 13: 221.
3. Cheang M C, Voduc D, Bajdik C, Leung S, McKinney S, Chia S K, et al. (2008) Basal-like breast cancer defined by five biomarkers has superior prognostic value than triple-negative phenotype. Clin Cancer Res 14: 1368-1376.
4. Mahauad-Fernandez W D, DeMali K A, Olivier A K, Okeoma C M (2014) Bone marrow stromal antigen 2 expressed in cancer cells promotes mammary tumor growth and metastasis. Breast Cancer Res 16: 493.
5. Sauter D (2014) Counteraction of the multifunctional restriction factor tetherin. Front Microbiol 5: 163.
6. Cole G, Simonetti K, Ademi I, Sharpe S (2012) Dimerization of the transmembrane domain of human tetherin in membrane mimetic environments. Biochemistry 51: 5033-5040.
7. Kupzig S, Korolchuk V, Rollason R, Sugden A, Wilde A, Banting G. (2003) BST-2/HM1.24 is a raft-associated apical membrane protein with an unusual topology. Traffic 4: 694-709.
8. Goto T, Kennel S J, Abe M, Takishita M, Kosaka M, Solomon A, et al. (1994) A novel membrane antigen selectively expressed on terminally differentiated human B cells. Blood 84: 1922-1930.
9. Neil S J, Zang T, Bieniasz P D (2008) Tetherin inhibits retrovirus release and is antagonized by HIV-1 Vpu. Nature 451: 425-430.
10. Van Damme N, Goff D, Katsura C, Jorgenson R L, Mitchell R, Johnson M C, et al. (2008) The interferon-induced protein BST-2 restricts HIV-1 release and is downregulated from the cell surface by the viral Vpu protein. Cell Host Microbe 3: 245-252.
11. Jones P H, Mehta H V, Maric M, Roller R J, Okeoma C M (2012) Bone marrow stromal cell antigen 2 (BST-2) restricts mouse mammary tumor virus (MMTV) replication in vivo. Retrovirology 9: 10.
12. Jones P H, Maric M, Madison M N, Maury W, Roller R J, Okeoma C M. (2013) BST-2/tetherin-mediated restriction of chikungunya (CHIKV) VLP budding is counteracted by CHIKV non-structural protein 1 (nsP1). Virology 438: 37-49.
13. Mahauad-Fernandez W D, Jones P H, Okeoma C M (2014) Critical role for BST-2 in acute Chikungunya virus infection. J Gen Virol.
14. Li S X, Barrett B S, Heilman K J, Messer R J, Liberatore R A, Bieniasz P D, et al. (2014) Tetherin promotes the innate and adaptive cell-mediated immune response against retrovirus infection in vivo. J Immunol 193: 306-316.
15. Galao R P, Le Tortorec A, Pickering S, Kueck T, Neil S J (2012) Innate sensing of HIV-1 assembly by Tetherin induces NFkappaB-dependent proinflammatory responses. Cell Host Microbe 12: 633-644.
16. Tokarev A, Suarez M, Kwan W, Fitzpatrick K, Singh R, Guatelli J. (2013) Stimulation of NF-kappaB activity by the HIV restriction factor BST2. J Virol 87: 2046-2057.
17. Arias J F, Heyer L N, von Bredow B, Weisgrau K L, Moldt B, Burton D R, et al. (2014) Tetherin antagonism by Vpu protects HIV-infected cells from antibody-dependent cell-mediated cytotoxicity. Proc Natl Acad Sci USA 111: 6425-6430.
18. Pham T N, Lukhele S, Hajjar F, Routy J P, Cohen E A (2014) HIV Nef and Vpu protect HIV-infected CD4+ T cells from antibody-mediated cell lysis through down-modulation of CD4 and BST2. Retrovirology 11: 15.
19. Alvarez R A, Hamlin R E, Monroe A, Moldt B, Hotta M T, Rodriguez Caprio G, et al. (2014) HIV-1 Vpu Antagonism of Tetherin Inhibits Antibody-Dependent Cellular Cytotoxic Responses by Natural Killer Cells. J Virol 88: 6031-6046.
20. Silveira N J, Varuzza L, Machado-Lima A, Lauretto M S, Pinheiro D G, Rodrigues R V, et al. (2008) Searching for molecular markers in head and neck squamous cell carcinomas (HNSCC) by statistical and bioinformatic analysis of larynx-derived SAGE libraries. BMC Med Genomics 1: 56.
21. Fang K H, Kao H K, Chi L M, Liang Y, Liu S C, Hseuh C, et al. (2014) Overexpression of BST2 is associated with nodal metastasis and poorer prognosis in oral cavity cancer. Laryngoscope.
22. Wainwright D A, Balyasnikova I V, Han Y, Lesniak M S (2011) The expression of BST2 in human and experimental mouse brain tumors. Exp Mol Pathol 91: 440-446.
23. Wang W, Nishioka Y, Ozaki S, Jalili A, Verma V K, Hanibuchi M, et al. (2009) Chimeric and humanized anti-HM1.24 antibodies mediate antibody-dependent cellular cytotoxicity against lung cancer cells. Lung Cancer 63: 23-31.
24. Wong Y F, Cheung T H, Lo K W, Yim S F, Siu N S, Chan S C, et al. (2007) Identification of molecular markers and signaling pathway in endometrial cancer in Hong Kong Chinese women by genome-wide gene expression profiling. Oncogene 26: 1971-1982.
25. Schliemann C, Roesli C, Kamada H, Borgia B, Fugmann T, Klapper W, et al. (2010) In vivo biotinylation of the vasculature in B-cell lymphoma identifies BST-2 as a target for antibody-based therapy. Blood 115: 736-744.
26. Cai D, Cao J, Li Z, Zheng X, Yao Y, Li W, et al. (2009) Up-regulation of bone marrow stromal protein 2 (BST2) in breast cancer with bone metastasis. BMC Cancer 9: 102.
27. Ono K, Ohtomo T, Yoshida K, Yoshimura Y, Kawai S, Koishihara Y, et al. (1999) The humanized anti-HM1.24 antibody effectively kills multiple myeloma cells by human effector cell-mediated cytotoxicity. Mol Immunol 36: 387-395.
28. Tai Y T, Horton H M, Kong S Y, Pong E, Chen H, Cemerski S, et al. (2012) Potent in vitro and in vivo activity of an Fc-engineered humanized anti-HM1.24 antibody against multiple myeloma via augmented effector function. Blood 119: 2074-2082.
29. Yi E H, Yoo H, Noh K H, Han S, Lee H, Won C, et al. (2013) BST-2 is a potential activator of invasion and migration in tamoxifen-resistant breast cancer cells. Biochem Biophys Res Commun 435: 685-690.
30. Sayeed A, Luciani-Torres G, Meng Z, Bennington J L, Moore D H, Dairkee S H. (2013) Aberrant regulation of the BST2 (Tetherin) promoter enhances cell proliferation and apoptosis evasion in high grade breast cancer cells. PLoS One 8: e67191.
31. Jovanovic J, Ronneberg J A, Tost J, Kristensen V (2010) The epigenetics of breast cancer. Mol Oncol 4: 242-254.
32. Razin A, Szyf M (1984) DNA methylation patterns. Formation and function. Biochim Biophys Acta 782: 331-342.
33. Rauch T A, Wu X, Zhong X, Riggs A D, Pfeifer G P (2009) A human B cell methylome at 100-base pair resolution. Proc Natl Acad Sci USA 106: 671-678.
34. Razin A, Cedar H (1977) Distribution of 5-methylcytosine in chromatin. Proc Natl Acad Sci USA 74: 2725-2728.
35. Ting A H, McGarvey K M, Baylin S B (2006) The cancer epigenome—components and functional correlates. Genes Dev 20: 3215-3231.
36. Schweiger M R, Barmeyer C, Timmermann B (2013) Genomics and epigenomics: new promises of personalized medicine for cancer patients. Brief Funct Genomics 12: 411-421.
37. Zhu J, Sanborn J Z, Benz S, Szeto C, Hsu F, Kuhn R M, et al. (2009) The UCSC Cancer Genomics Browser. Nat Methods 6: 239-240.
38. Cline M S, Craft B, Swatloski T, Goldman M, Ma S, Haussler D, et al. (2013) Exploring TCGA Pan-Cancer data at the UCSC Cancer Genomics Browser. Sci Rep 3: 2652.
39. Weinstein J N, Collisson E A, Mills G B, Shaw K R, Ozenberger B A, Ellrott K, et al. (2013) The Cancer Genome Atlas Pan-Cancer analysis project. Nat Genet 45: 1113-1120.
40. Casey T, Bond J, Tighe S, Hunter T, Lintault L, Patel O, et al. (2009) Molecular signatures suggest a major role for stromal cells in development of invasive breast cancer. Breast Cancer Res Treat 114: 47-62.
41. Riaz M, van Jaarsveld M T, Hollestelle A, Prager-van der Smissen W J, Heine A A, Boersma A W, et al. (2013) miRNA expression profiling of 51 human breast cancer cell lines reveals subtype and driver mutation-specific miRNAs. Breast Cancer Res 15: R33.
42. Yamamoto S, Wu Z, Russnes H G, Takagi S, Peluffo G, Vaske C, et al. (2014) JARID1B is a luminal lineage-driving oncogene in breast cancer. Cancer Cell 25: 762-777.
43. Radpour R, Barekati Z, Kohler C, Schumacher M M, Grussenmeyer T, Jenoe P, et al. (2011) Integrated epigenetics of human breast cancer: synoptic investigation of targeted genes, microRNAs and proteins upon demethylation treatment. PLoS One 6: e27355.
44. Putnik M, Zhao C, Gustafsson J A, Dahlman-Wright K (2012) Global identification of genes regulated by estrogen signaling and demethylation in MCF-7 breast cancer cells. Biochem Biophys Res Commun 426: 26-32.
45. Jones P H, Mahauad-Fernandez W D, Madison M N, Okeoma C M (2013) BST-2/tetherin is overexpressed in mammary gland and tumor tissues in MMTV-induced mammary cancer. Virology 444: 124-139.
46. Jones P H, Mehta H V, Okeoma C M (2012) A novel role for APOBEC3: susceptibility to sexual transmission of murine acquired immunodeficiency virus (mAIDS) is aggravated in APOBEC3 deficient mice. Retrovirology 9: 50.
47. Mehta H V, Jones P H, Weiss J P, Okeoma C M (2012) IFN-alpha and lipopolysaccharide upregulate APOBEC3 mRNA through different signaling pathways. J Immunol 189: 4088-4103.
48. Madison M N, Roller R J, Okeoma C M (2014) Human semen contains exosomes with potent anti-HIV-1 activity. Retrovirology 11: 102.
49. Mehta H V, Jones P H, Weiss J P, Okeoma C M (2012) IFN-alpha and Lipopolysaccharide Upregulate APOBEC3 mRNA through Different Signaling Pathways. J Immunol.
50. Jones P H, Okeoma C M (2013) Phosphatidylinositol 3-kinase is involved in Toll-like receptor 4-mediated BST-2/Tetherin regulation. Cell Signal.
51. Tao Y, Liu S, Briones V, Geiman T M, Muegge K (2011) Treatment of breast cancer cells with DNA demethylating agents leads to a release of Pol II stalling at genes with DNA-hypermethylated regions upstream of TSS. Nucleic Acids Res 39: 9508-9520.
52. Gius D, Cui H, Bradbury C M, Cook J, Smart D K, Zhao S, et al. (2004) Distinct effects on gene expression of chemical and genetic manipulation of the cancer epigenome revealed by a multimodality approach. Cancer Cell 6: 361-371.
53. Schuebel K E, Chen W, Cope L, Glockner S C, Suzuki H, Yi M, et al. (2007) Comparing the DNA hypermethylome with gene mutations in human colorectal cancer. PLoS Genet 3: 1709-1723.
54. Gama-Sosa M A, Slagel V A, Trewyn R W, Oxenhandler R, Kuo K C, Gehrke C W, et al. (1983) The 5-methylcytosine content of DNA from human tumors. Nucleic Acids Res 11: 6883-6894.
55. de Capoa A, Musolino A, Della Rosa S, Caiafa P, Mariani L, Del Nonno F, et al. (2003) DNA demethylation is directly related to tumour progression: evidence in normal, pre-malignant and malignant cells from uterine cervix samples. Oncol Rep 10: 545-549.
56. Bedford M T, van Helden P D (1987) Hypomethylation of DNA in pathological conditions of the human prostate. Cancer Res 47: 5274-5276.
57. Herman J G, Baylin S B (2003) Gene silencing in cancer in association with promoter hypermethylation. N Engl J Med 349: 2042-2054.
58. Ferguson A T, Vertino P M, Spitzner J R, Baylin S B, Muller M T, Davidson N E. (1997) Role of estrogen receptor gene demethylation and DNA methyltransferase. DNA adduct formation in 5-aza-2′deoxycytidine-induced cytotoxicity in human breast cancer cells. J Biol Chem 272: 32260-32266.
59. Ehrlich M (2002) DNA hypomethylation, cancer, the immunodeficiency, centromeric region instability, facial anomalies syndrome and chromosomal rearrangements. J Nutr 132: 2424s-2429s.
60. Ehrlich M, Woods C B, Yu M C, Dubeau L, Yang F, Campan M, et al. (2006) Quantitative analysis of associations between DNA hypermethylation, hypomethylation, and DNMT RNA levels in ovarian tumors. Oncogene 25: 2636-2645.
61. Son K S, Kang H S, Kim S J, Jung S Y, Min S Y, Lee S Y, et al. (2010) Hypomethylation of the interleukin-10 gene in breast cancer tissues. Breast 19: 484-488.
62. Wang Z, Zhang J, Zhang Y, Lim S H (2006) SPAN-Xb expression in myeloma cells is dependent on promoter hypomethylation and can be upregulated pharmacologically. Int J Cancer 118: 1436-1444.
63. (2012) Comprehensive molecular portraits of human breast tumours. Nature 490: 61-70.
64. Kawai S, Azuma Y, Fujii E, Furugaki K, Ozaki S, Matsumoto T, et al. (2008) Interferon-alpha enhances CD317 expression and the antitumor activity of anti-CD317 monoclonal antibody in renal cell carcinoma xenograft models. Cancer Sci 99: 2461-2466.
65. Bego M G, Mercier J, Cohen E A (2012) Virus-activated interferon regulatory factor 7 upregulates expression of the interferon-regulated BST2 gene independently of interferon signaling. J Virol 86: 3513-3527.
66. Varley K E, Gertz J, Bowling K M, Parker S L, Reddy T E, Pauli-Behn F, et al. (2013) Dynamic DNA methylation across diverse human cell lines and tissues. Genome Res 23: 555-567.
67. Cui W, Wang L H, Wen Y Y, Song M, Li B L, Chen X L, et al. (2010) Expression and regulation mechanisms of Sonic Hedgehog in breast cancer. Cancer Sci 101: 927-933.
68. Milicic A, Harrison L A, Goodlad R A, Hardy R G, Nicholson A M, Presz M, et al. (2008) Ectopic expression of P-cadherin correlates with promoter hypomethylation early in colorectal carcinogenesis and enhanced intestinal crypt fission in vivo. Cancer Res 68: 7760-7768.
69. Hibi K, Goto T, Mizukami H, Kitamura Y H, Sakuraba K, Sakata M, et al. (2009) Demethylation of the CDH3 gene is frequently detected in advanced colorectal cancer. Anticancer Res 29: 2215-2217.
70. Etcheverry A, Aubry M, de Tayrac M, Vauleon E, Boniface R, Guenot F, et al. (2010) DNA methylation in glioblastoma: impact on gene expression and clinical outcome. BMC Genomics 11: 701.
71. Coit P, Jeffries M, Altorok N, Dozmorov M G, Koelsch K A, Wren J D, et al. (2013) Genome-wide DNA methylation study suggests epigenetic accessibility and transcriptional poising of interferon-regulated genes in naive CD4+ T cells from lupus patients. J Autoimmun 43: 78-84.

### Example 3—Inhibition of BST-2 Dimerization or BST-2 Expression as Treatment for Breast Cancer and Viral Carcinogenesis

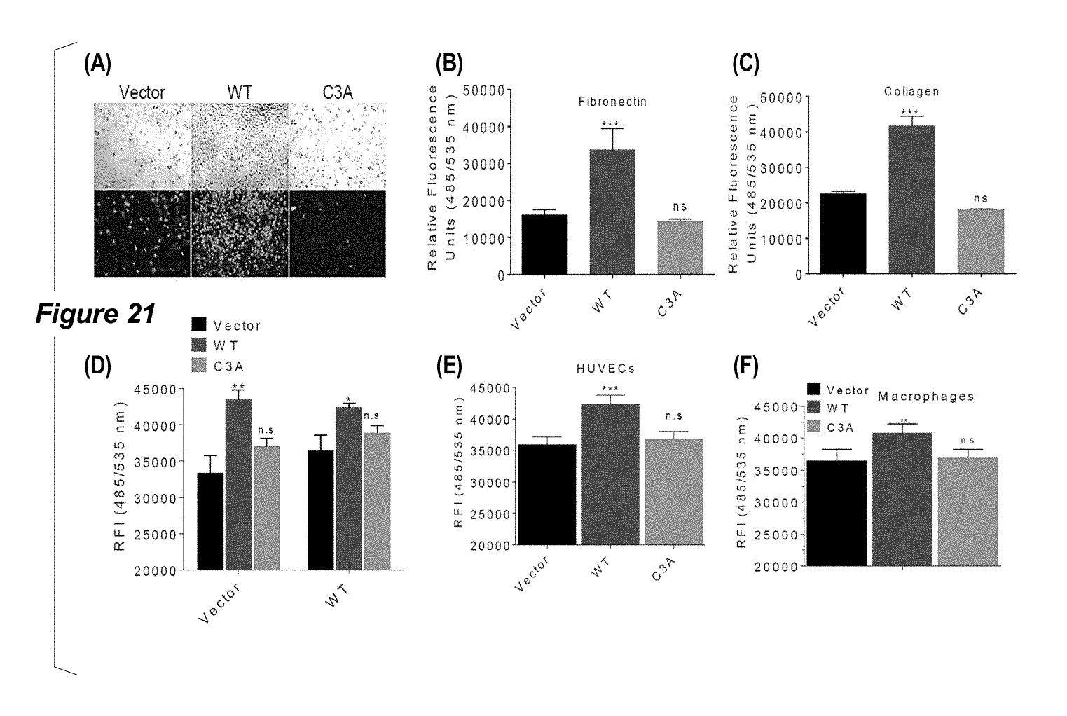

Covalent Dimerization of BST-2 Mediates Adhesion of Cancer Cells to ECM Proteins and Other Cells (FIG. 21).

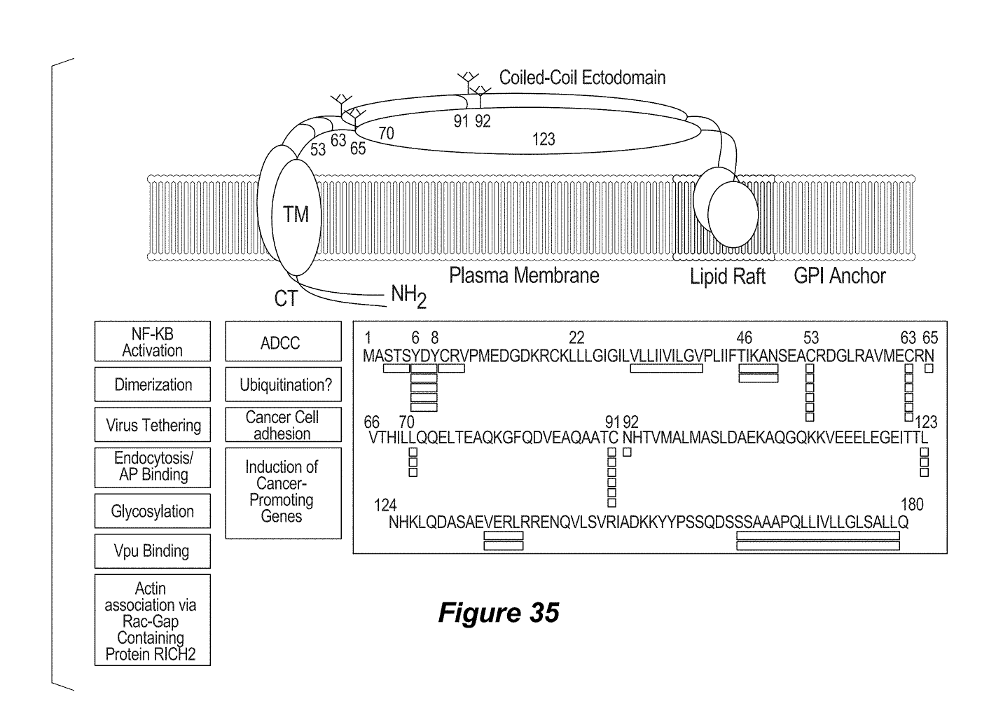

The ectodomain of BST-2 mediates cancer cell adhesion to extracellular matrix proteins, notably fibronectin and collagen. The ectodomain of BST-2 is responsible for cancer cell clustering. Such clustering may involve cancer cell to cancer cell, cancer cell to endothelial cells, and cancer cells to immune cells (FIG. 21). These results suggest that the ability of BST-2 to form stable dimers, as mediated by the three cysteines (53, 63, and 91) located in the BST-2 ECD) (FIG. 35) promotes cell to cell interaction that may allow collective cancer cell clustering in vivo. This process will result in survival of cancer cells expressing BST-2 during transit to metastatic sites in the host.

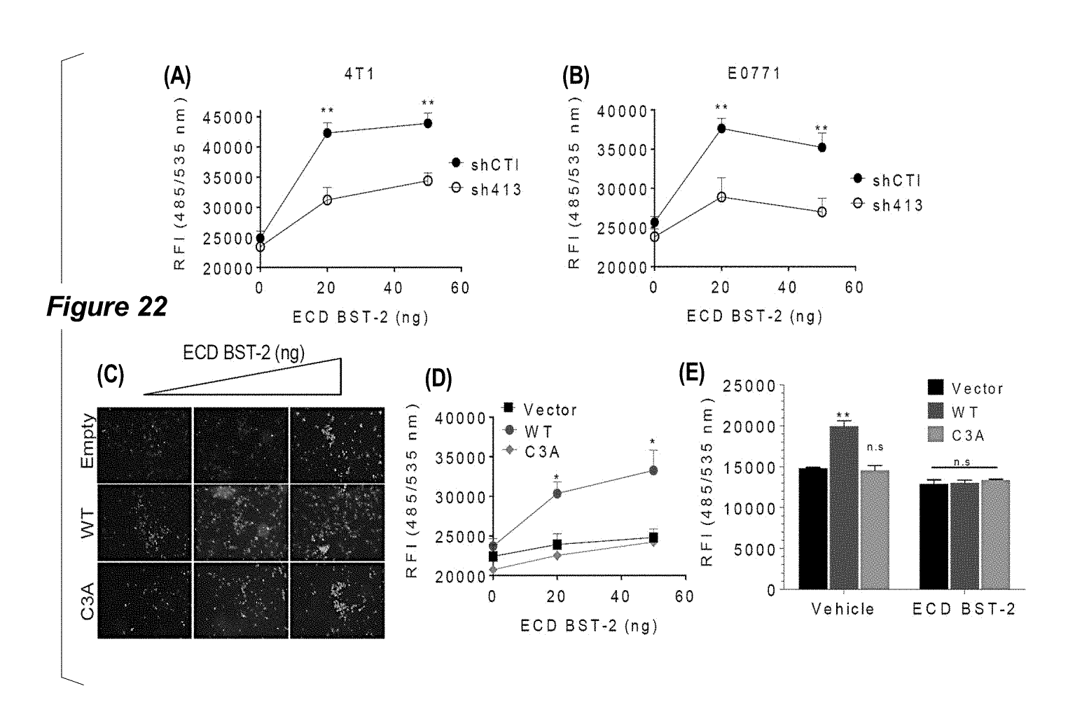

Recombinant Extracellular Domain (ECD) of BST-2 Binds to BST-2 In Vitro and Prevents BST-2-Mediated Adhesion of Breast Cancer Cells (FIG. 22).

Breast cancer BST-2 expressing cells (shCTL) efficiently bind BST-2 ECD while cells suppressed of BST-2 (sh413) do not bind efficiently (FIG. 22). Low BST-2 expressing human breast cancer cells (MCF-7) stably expressing WT BST-2 (WT) efficiently binds BST-2 ECD while similar cells stably expressing dimerization mutant BST-2 (C3A) are defective in binding BST-2 ECD. Treatment of BST-2-expressing breast cancer cells with BST-2 ECD blocks cancer cell-to-cancer clustering (FIG. 22). These findings suggest that recombinant peptide that specifically binds to the ECD of BST-2 shows efficacy in binding BST-2 expressed in cancer cells, thus reducing the ability of the cancer cells to cluster. This shows the potential of the BST-2 ECD as an inhibitor of BST-2 dimerization.

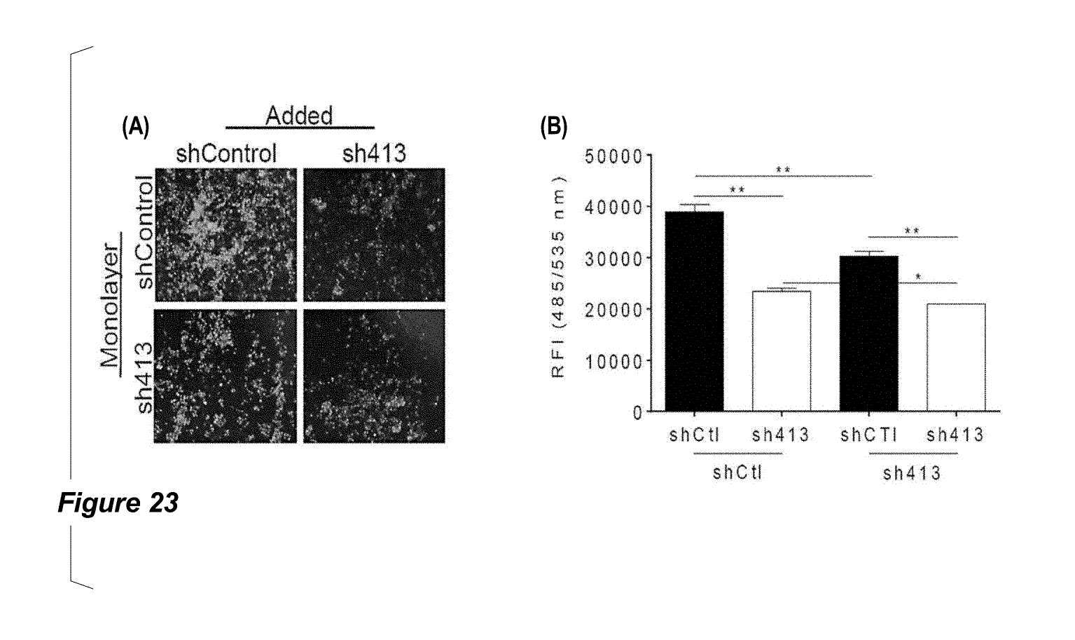

BST-2 in Cancer Cells Mediates Cancer Cell Adhesion to Each Other, Resulting in Cancer Cell Clustering (FIG. 23).

The process of cancer cell clustering promotes cancer cell tumorigenesis since cell clustering during metastasis (transit in the vessels) increases cancer cell survival.

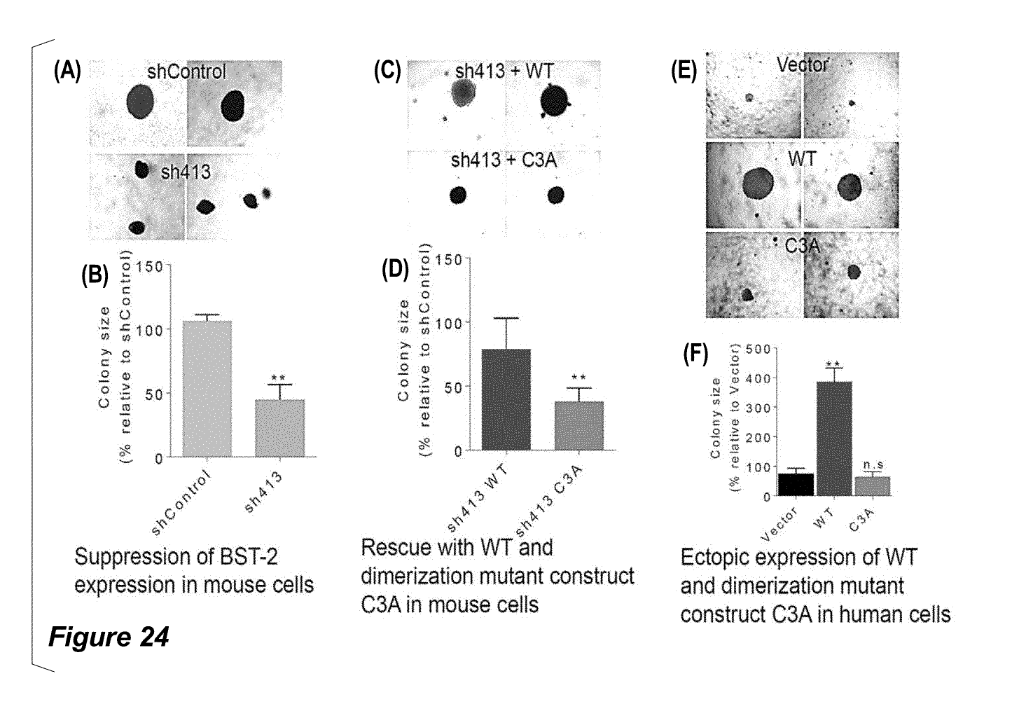

Covalent Dimerization of BST-2 is Important for Anchorage-Independent Growth of Breast Cancer Cells (FIG. 24).

Suppression of BST-2 in breast cancer cells (sh413) inhibits colony formation by cancer cells. Rescue of BST-2 expression in BST-2 suppressed sh413 cells with WT BST-2 rescued colony formation but expression of dimerization mutant BST-2 (C3A) had no effect on colony formation. In addition, overexpression of WT BST-2 but not BST-2 C3A in MCF-7 cells with low BST-2 enhanced colony formation (FIG. 24). Therefore, BST-2 dimerization mediated by BST-2 ECD promotes anchorage-independent growth of cancer cells, thus allowing cancer cells to form colonies. This process could be mediated by cancer cell adhesion or by resistance of cancer cells to anoikis.

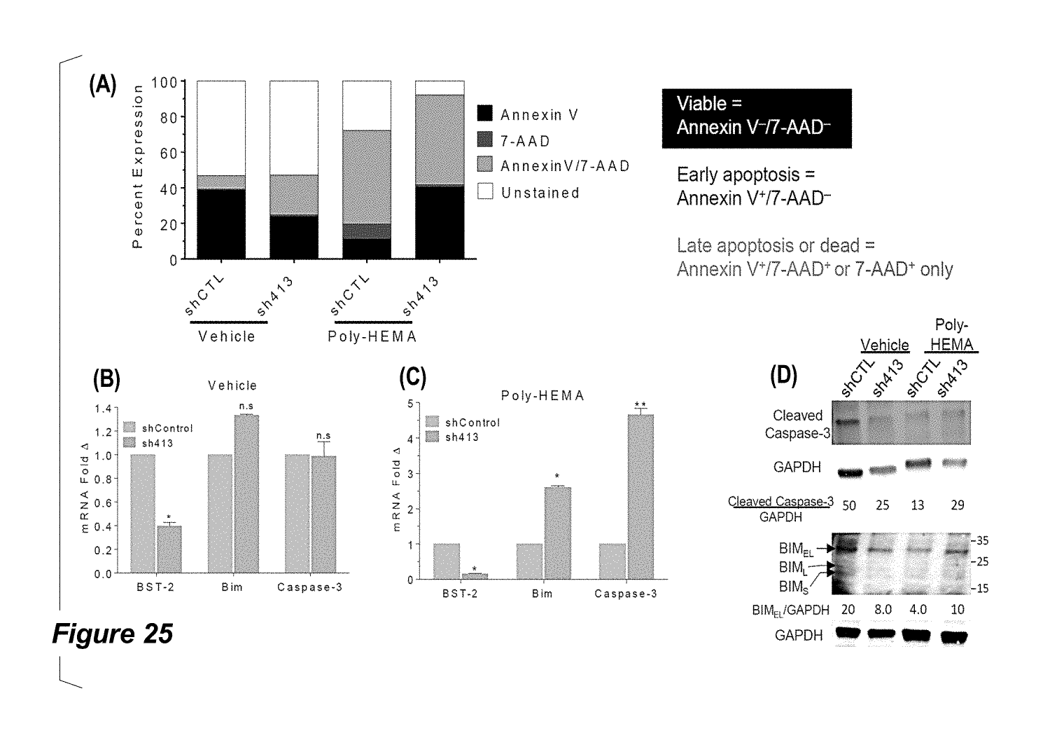

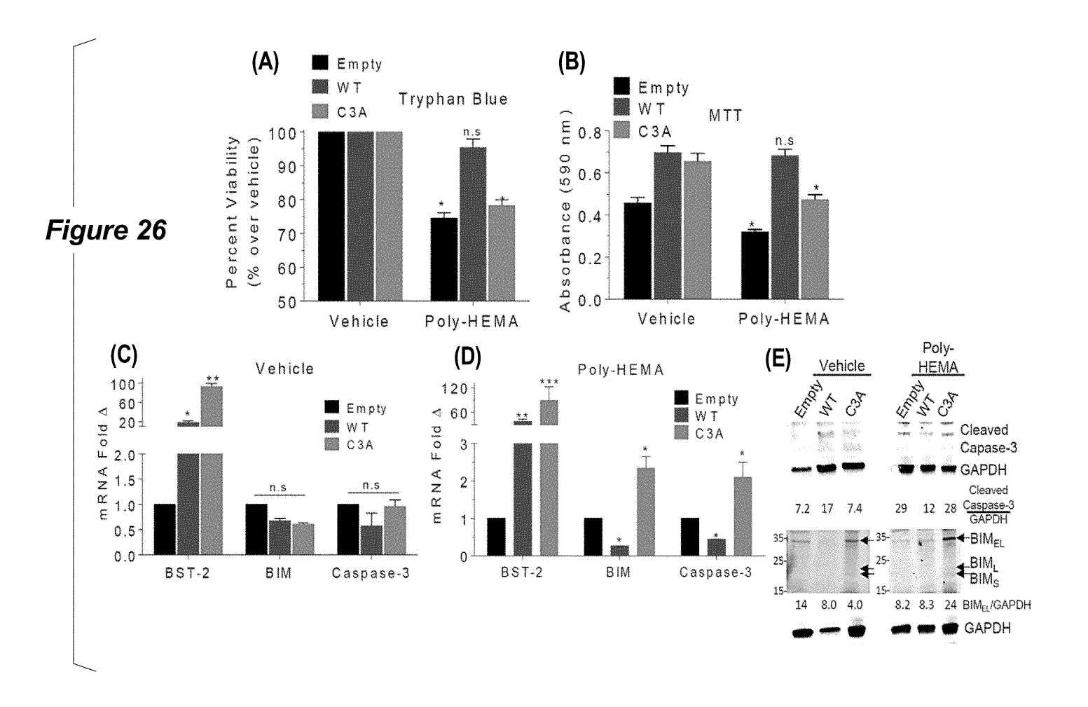

BST-2 Dimerization Renders Cancer Cells Resistance to Anoikis Via Downregulation of BIM (FIGS. 25 and 26).

Breast cancer cells expressing BST-2 are resistant to anoikis (detachment-induced cell death). However, suppression of BST-2 renders cells susceptible to anoikis. Under normal condition, BST-2 expression has no effect on cancer cell death as evidenced by similar levels of the apoptotic factors Bim and Caspase-3. However, under anoikis condition, BST-2 expression promotes cancer cell survival and loss of BST-2 renders cells susceptible to cell death as evidenced by higher levels of the apoptotic factors Bim and Caspase-3 in sh413 cells (FIG. 25). Thus, BST-2 is important in keeping tumor cells that detached from the primary tumors alive. This property of BST-2 promotes metastasis. Moreover, overexpression of WT BST-2 in low BST-2 expressing breast cancer cells renders cancer cells resistant to anoikis. However, overexpression of dimerization mutant BST-2 C3A does not protect cells from anoikis (FIG. 26). Under normal condition, BST-2 expression or dimerization (WT and C3A) has no effect on cancer cell death as evidenced by similar levels of the apoptotic factors Bim and Caspase-3. Under anoikis condition, expression of WT BST-2 but not BST-2 C3A promotes cancer cell survival as evidenced by increased levels of the apoptotic factors Bim and Caspase-3 in BST-2 C3A cells (FIG. 26). Thus, the ECD of BST-2 is responsible for BST-2 mediated resistance of cancer cells to anoikis, suggesting that C3A tumors may not grow.

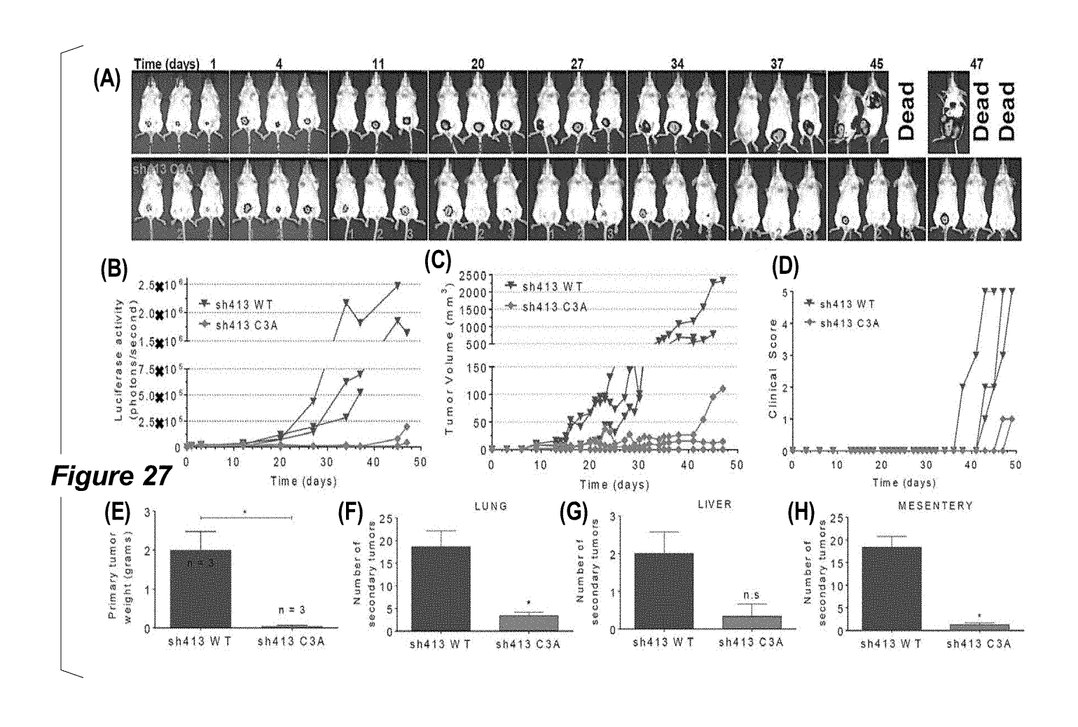

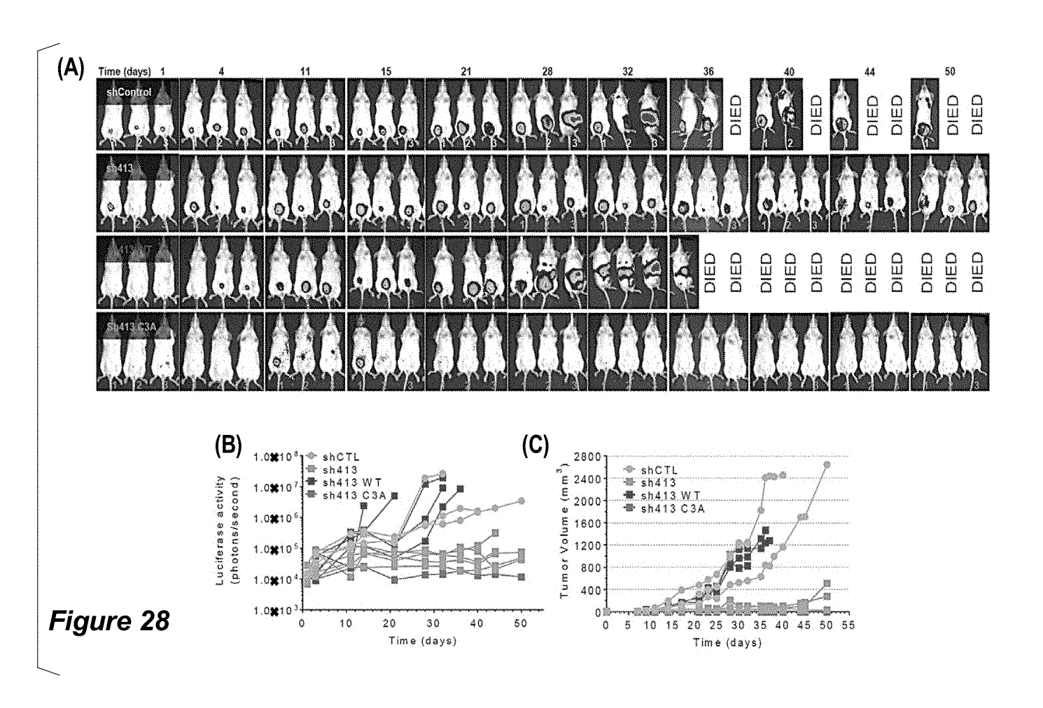

Breast Cancer Cells that Cannot Form BST-2 Dimers are Deficient in Breast Tumor Formation (FIGS. 27 and 28).

BALB/c mice orthotopically injected with equivalent numbers of luciferase expressing sh413 cells in which BST-2 was rescued with WT BST-2 (sh413WT) or dimerization mutant BST-2 (sh413C3A) were monitored for tumor growth and metastasis by IVIS imaging of luciferase expression over 47 days. Results show that the ECD of BST-2 controls the ability of BST-2 to promote primary breast tumor growth and metastatic spread of tumors to the lungs mesentery, and possibly other sites. C3A tumors failed to grow. Furthermore, BALB/c mice orthotopically injected with equivalent numbers of luciferase expressing shControl, sh413 or sh413 cells in which BST-2 was rescued with WT BST-2 (sh413WT) or dimerization mutant BST-2 (sh413C3A) were monitored for tumor growth and metastasis by IVIS imaging of luciferase expression over 50 days (FIGS. 27 and 28). Tumor volume was measured overtime. Results show that, the ECD of BST-2 controls the ability of BST-2 to promote primary breast tumor growth and overall morbidity in cancer cell injected mice. Primary tumors regressed sh413C3A injected mice (FIGS. 27, 28 and 29).

Breast Cancer Cells that Cannot Form BST-2 Dimers have Decreased Metastatic Capacity Resulting in Increased Host Survival (FIGS. 27, 28 and 29).

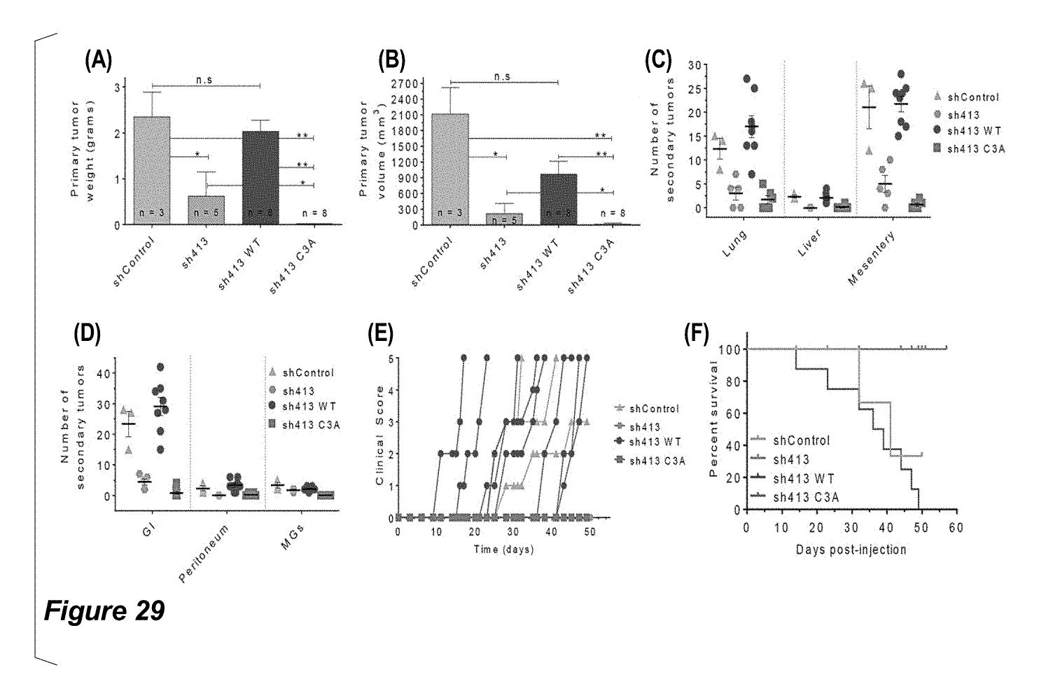

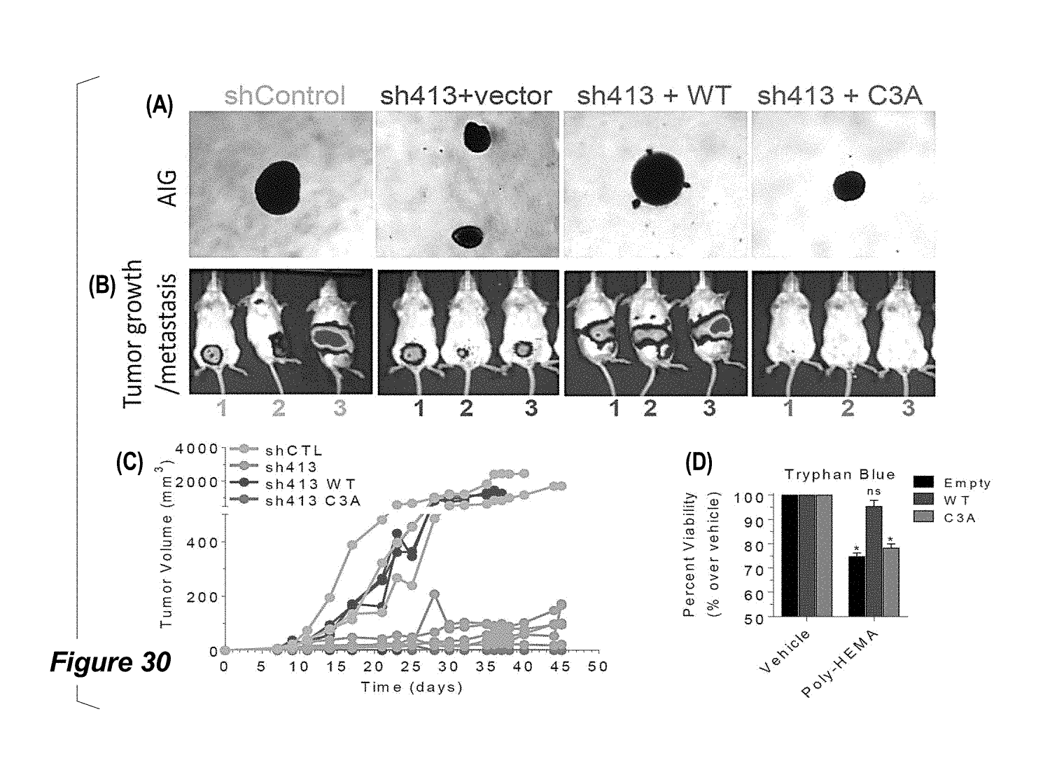

Weight and final volume of primary tumors from BALB/c mice orthotopically injected with equivalent numbers of luciferase expressing shControl, sh413 or sh413 cells in which BST-2 was rescued with WT BST-2 (sh413WT) or dimerization mutant BST-2 (sh413C3A). Number of secondary tumors from shControl, sh413, sh413WT and sh413C3A injected BALB/c mice and Clinical score of tumor bearing mice was recorded (FIG. 29). Survival plot of shControl, sh413, sh413WT and sh413C3A injected BALB/c mice was also recorded (FIG. 29). Thus, the ECD of BST-2 controls the ability of BST-2 to promote primary breast tumor growth and metastatic spread of tumors to the lung, mesentery, GI tract and peritoneum. sh413 and sh413C3A injected mice did not succumb to disease. Thus, the ECD of BST-2 controls collective cell clustering and promotes primary breast tumor growth and metastatic spread of tumors (FIG. 30).

### Example 4—Identification of BST-2 Based Peptide that Inhibits Breast Tumor Growth and Metastasis

Results

Synthesis of BST-2 Peptides.

WT BST-2 peptide synthesis was performed by Selleck. The synthesized peptide encompasses amino acids 47-95 of the BST-2 protein and contains cell penetrating molecule (penetratin) at the N-terminus. C3A BST-2 peptide encompasses amino acids 47-95 of the BST-2 protein and contains cell penetrating molecule (penetratin) at the N-terminus. Cysteines at positions 53, 63, and 91 were replaced with alanines in the C3A Peptide.

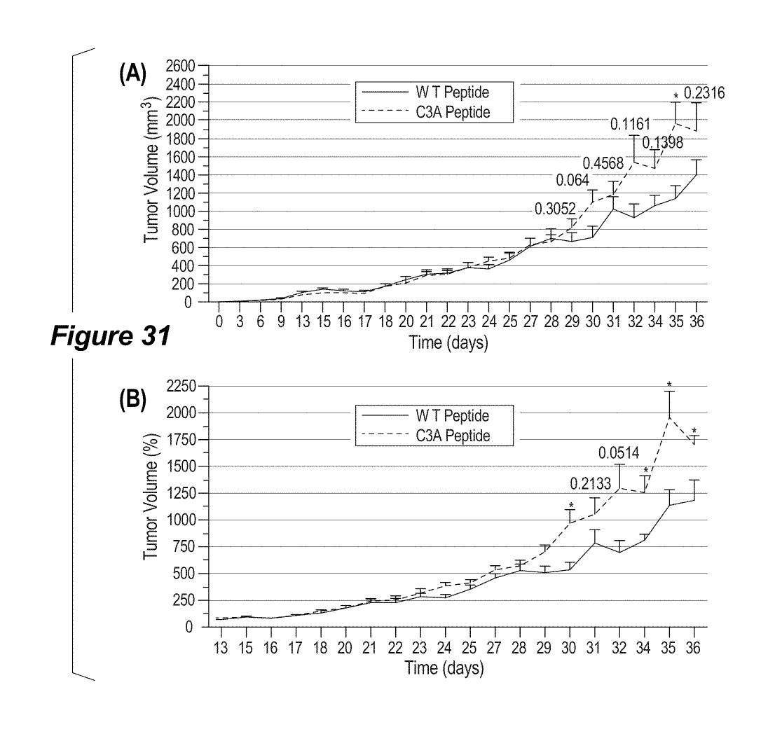

Treating Tumor Bearing Mice with WT Peptide Results in Slower Tumor Growth (FIG. 31).

Tumor bearing mice were treated intratumorally with WT or C3A peptides when tumor volume reached 100 mm³. Treatment was performed every 3 days. Tumor volumes were measured daily (panel A) with the formula TV=0.5(Length*Width2). Tumor volume presented as a percent was calculated by multiplying the tumor volume in a particular day by 100 and dividing it by the tumor volume on the first day of treatment to account for any variation in pre-treatment tumor volume. TV (%), (TV on day X*100/TV on day 1 of treatment). We found that our peptide that binds to the BST-2 ECD is a good target for inhibiting breast tumor growth. The level of tumor inhibition is remarkable given that these are crude peptides that have not been stabilized by modifications. Studies on peptide Pharmacokinetic, Pharmacodynamic, and Biodistribution are required to enhance antitumor efficacy of this treatment.

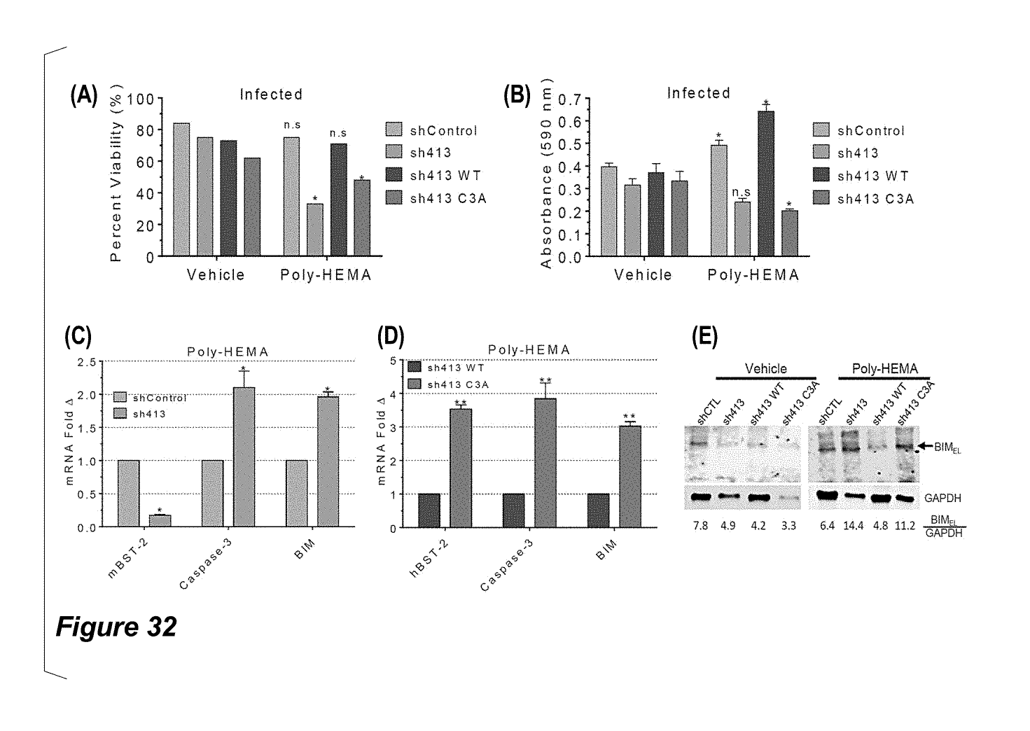

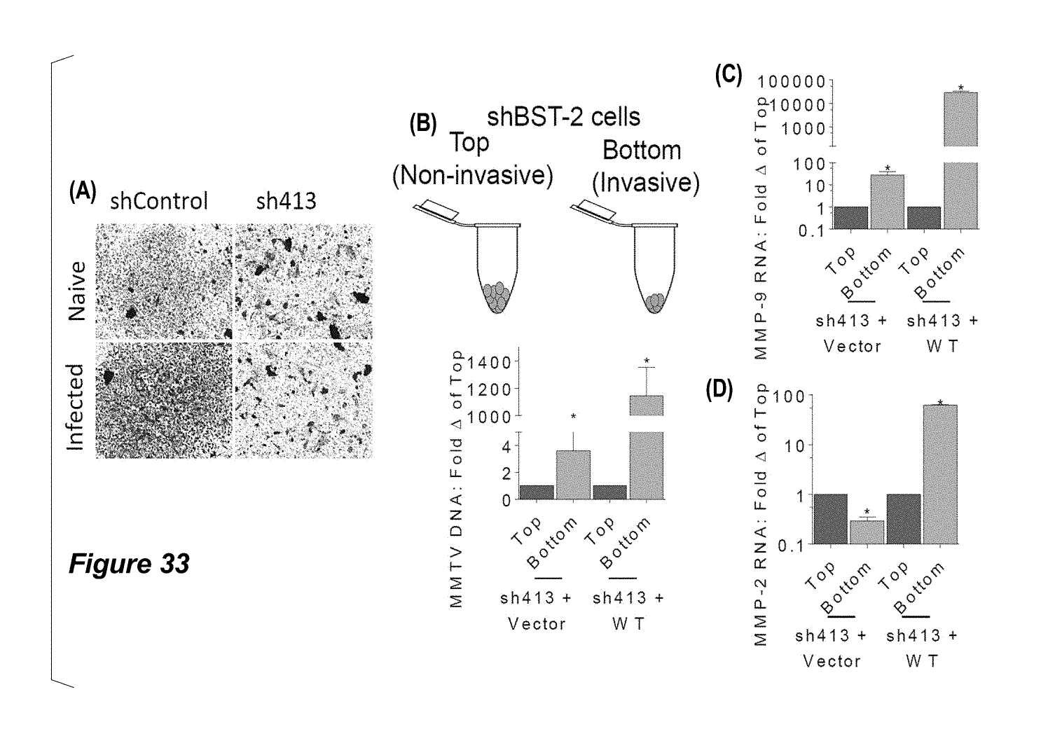

BST-2 Dimerization Controls Virus-Induced Cancer Cell Resistance to Anoikis and Invasion Through Matrigel (FIGS. 32 and 33).

MMTV promotes resistance to anoikis in BST-2-dimerization dependent manner via downregulation of BIM (apoptotic factor) (FIG. 32). Additionally, MMTV enhances cancer cell invasion in a BST-2-dependent manner by inducing the gelatinases MMP-2 and MMP-9 (FIG. 33). These are potential mechanisms that explain virus-induced resistance to anoikis and invasion of virus-infected cells.

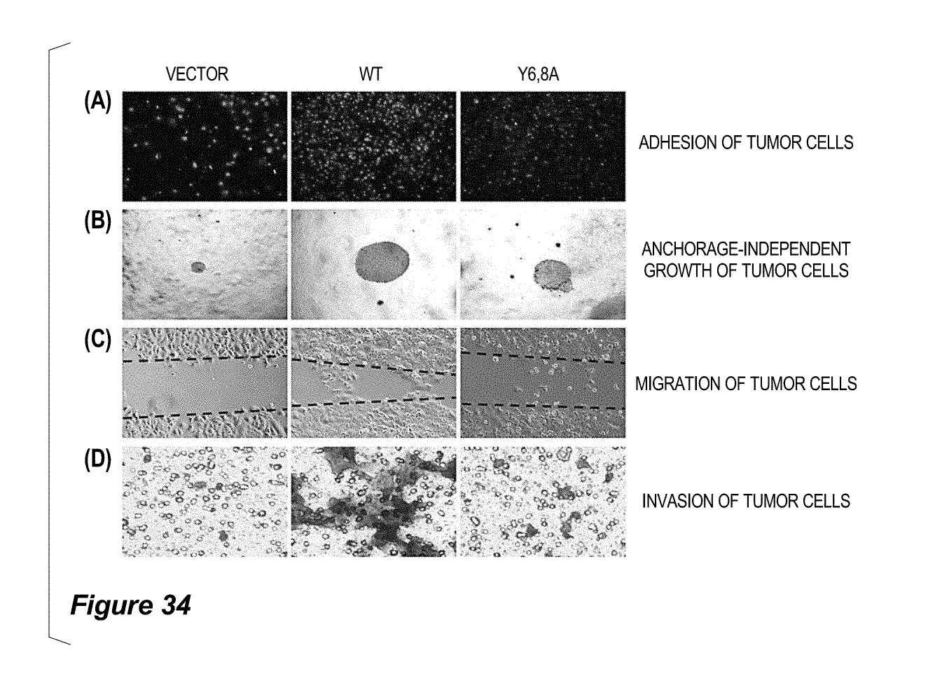

The Invasion Promoting Capacity of BST-2 in Breast Cancer Cells May be Mediated Through a Y×Y Motif Located on its Cytoplasmic Tail (FIG. 34 and FIG. 35).

Cells expressing a BST-2 mutant in which the Y×Y motif was mutated (Y6,8A) fail to migrate and invade properly; however, anchorage-independent growth and adhesion of cancer cell to fibronectin are not significantly affected by the lack of this motif. These data suggest that BST-2 Y×Y motif is involved in cancer cell migration and invasion, and pertubation of this motif may have great therapeutic implication. BST-2 Y×Y motif may be important in signal transduction activity that as has been shown for virus infected cells.

### Example 5—Generation of BST-2-Overexpressing Cancer Cells

4T1 sh413 cells which express low BST-2 levels were transfected with either pcDNA3.1 (sh413), with pcDNA3.1 containing WT human BST-2 (sh413 WT) or with pcDNA3.1 containing a mutant BST-2 were cysteines involved in dimerization at positions 53, 63 and 91 were replaced with alanines (sh413 C3A). These constructs are a kind gift from Dr. John Guatelli of UCSD and Dr. Klaus Strebel of NIH. Lipofectamine 2000 (Life technologies) was used for the transfections and the amounts used were adjusted according to the manufacturers' instructions. Transfected cells were selected with G418 at 500 μg/ml and stable cells were used in all experiments.

### Example 6—in Silico Analyses of BST-2 Levels and Circulating Tumor Cell Cluster Formation

Meta-analyses were performed as follows. The publically available Gene Expression Omnibus (GEO) dataset GSE51827 (see Aceto et al. (2014) Circulating tumor cell clusters are oligoclonal precursors of breast cancer metastasis. Cell 158: 1110-1122), which contains RNAseq data from circulating tumor cells (CTCs) singlets and clusters was used to determine the levels of BST-2 in cell populations. RPKM units were calculated using the formula: RPKM=(10⁹*Read Counts)/(Total mapped reads*Exon length). Intrapatient comparisons were performed by subtracting CTC singlets BST-2 levels from CTC clusters BST-2 levels of the same patient.

### Results and Conclusion

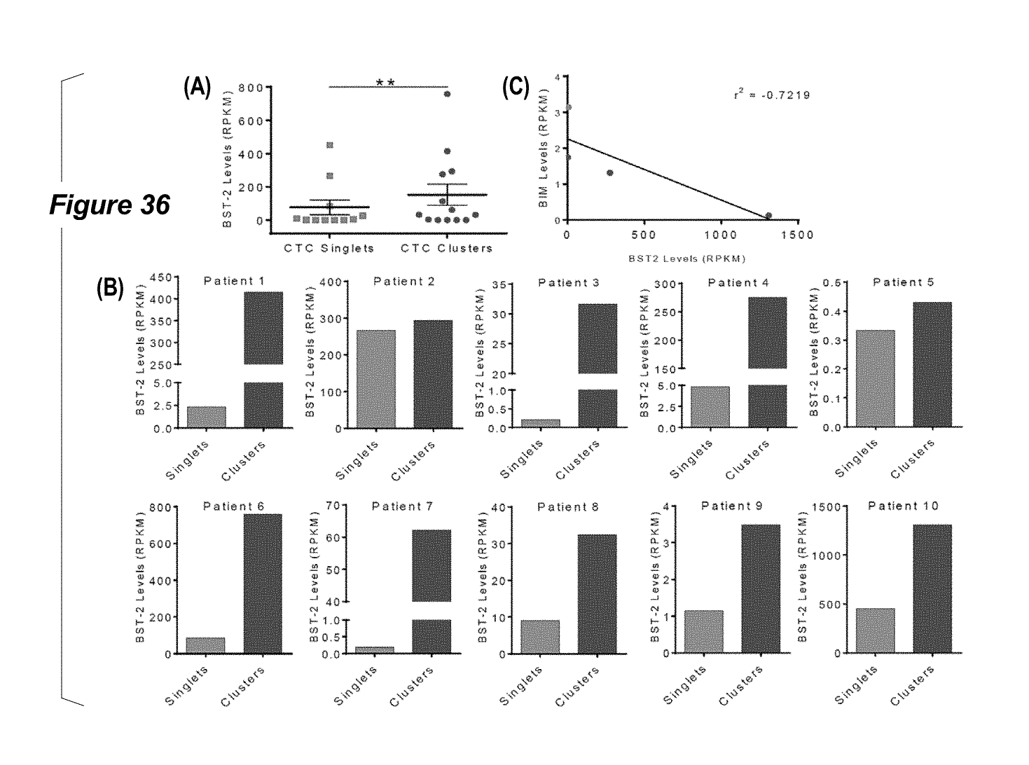

Results are illustrated in FIG. 36. We conclude that BST-2 is highly expressed in CTCs with elevated metastatic potential. We also conclude that BST-2 may promote the formation of circulating tumor cell clusters, resulting in increased metastatic potential of breast cancer cells, and BST-2 in circulating tumor cells may be useful as a predictive or prognostic bio-signature for breast cancer.

### Example 7—Mechanism of BST-2-Mediated Anoikis Resistance

Anoikis Assay and Cell Viability Analyses.

96-well plates were coated with 50 ul of sterile 95% Ethanol or 50 ul of 12 mg/ml Poly-HEMA in 95% Ethanol (Sigma-Aldrich) and allowed to dry for 72 hours under the hood as previously described. (See Phung et al. (2011) Rapid generation of in vitro multicellular spheroids for the study of monoclonal antibody therapy. J Cancer 2: 507-514). Poly-HEMA prevents cells from attaching to the plastic. Following, 4T1 shControl, sh413, sh413 WT, or sh413 C3A cells were plated at 20,000 cells/well. Plates were centrifuged at 1,200 g for 10 minutes and then incubated at 37° C. for 48 hours. Cells were collected to test cell viability using trypan blue (Life technologies) and an MTT assay (Life technologies). The rest of the cells were pelleted and kept at −20° C. until used for RNA and protein isolation.

Western Blots.

Western blots were performed as previously described. (See Jones et al., (2012) A novel role for APOBEC3: Susceptibility to sexual transmission of murine acquired immunodeficiency virus (mAIDS) is aggravated in APOBEC3 deficient mice. Retrovirology 9: 50; Mehta et al., (2012) IFN-alpha and Lipopolysaccharide Upregulate APOBEC3 mRNA through Different Signaling Pathways. J Immunol 189: 4088-4103; and Okeoma et al., (2010) APOBEC3 proteins expressed in mammary epithelial cells are packaged into retroviruses and can restrict transmission of milk-borne virions. Cell Host Microbe 8: 534-543). Briefly, protein extracts from 4T1 cells expressing various BST-2 constructs (WT, or C3A) were isolated, protein quantified using a Bradford assay and protein run in a western blot blotting for total levels of BIM and activated Caspase-3 (Cleaved caspase-3).

MTT Assay.

A total of 10,000 cells stably expressing Vector, WT, or C3A MCF-7 cells were plated in 96-well plates. Cells were then incubated with 5 mg/ml MTT reagent for 3.5 hours followed by addition of MTT solvent (0.1% NP-40 and 4 mM HCl in isopropanol) and rocking for 15 minutes. Absorbance at 590 nm was read using a Tecan Infinite M200 Pro plate reader.

### Results and Conclusion

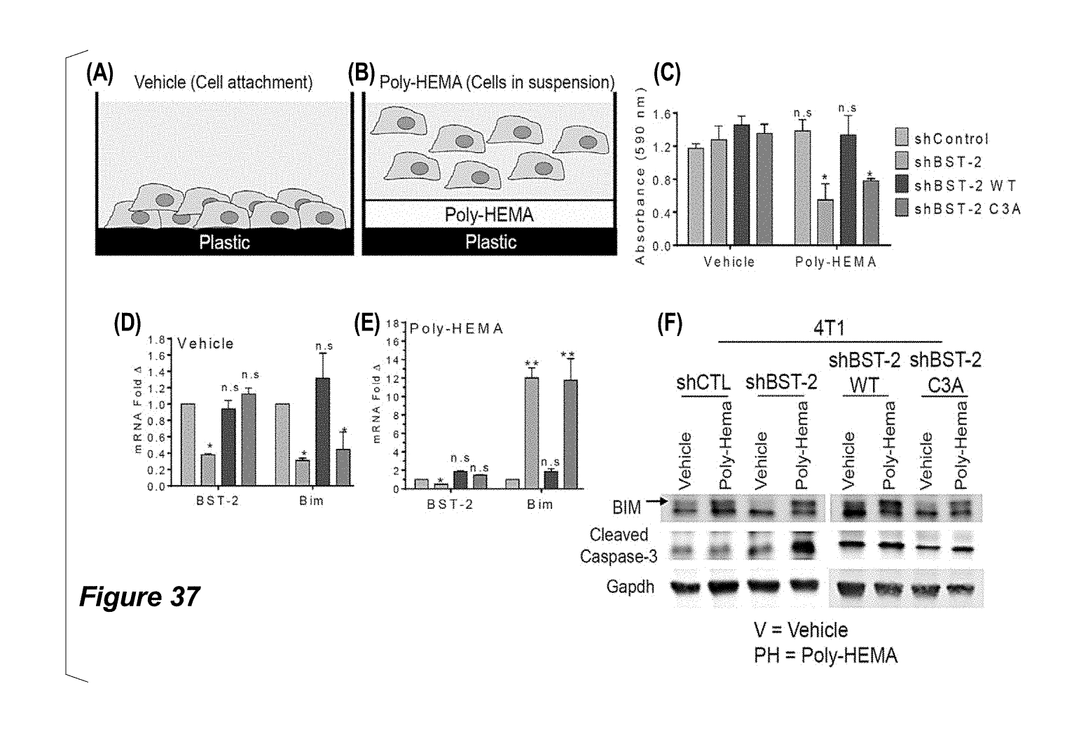

BST-2 promotes survival of breast cancer cells by endowing cancer cells with resistance to anoikis (FIG. 37). We conclude that suppression of BST-2 expression or disruption of BST-2 dimerization in cancer cells results in cancer death; which ultimately leads to decreased tumor growth and metastasis.

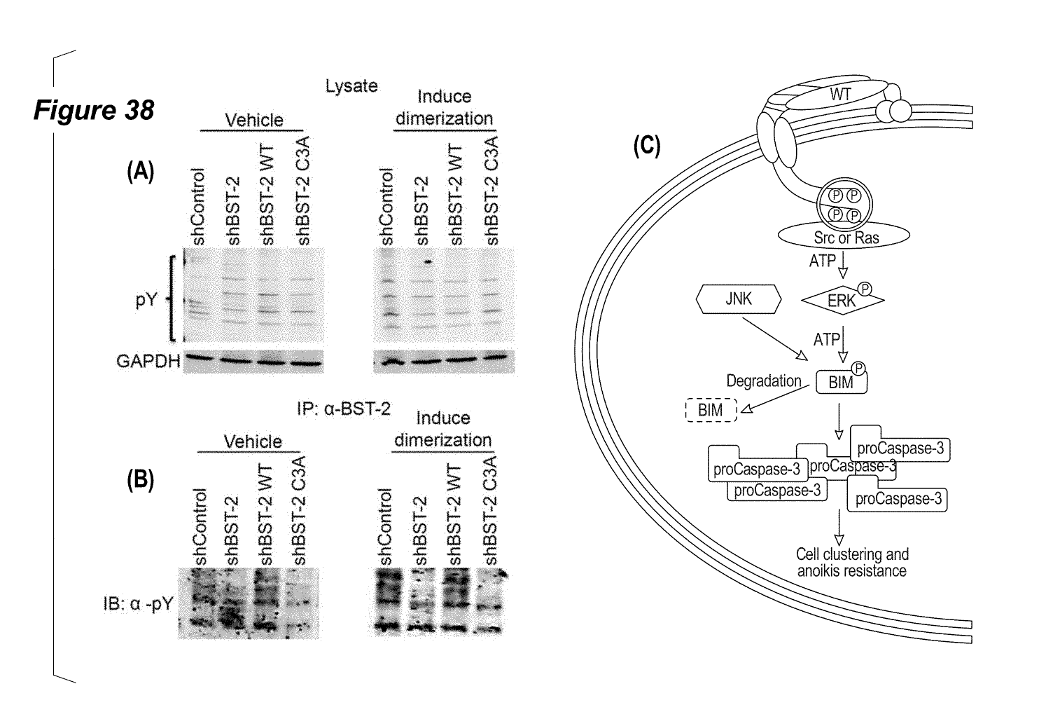

Equivalent numbers (300,000 cells) of 4T1 cells stably expressing non-targeting shRNA—shControl, BST-2-targeting shRNA—sh413, and sh413 cells rescued for BST-2 expression by stably expressing wild type BST-2—sh413 WT, or a dimerization defective BST-2—sh413 C3A were plated on 6-well plates. 4 hours later, cells were treated with PBS (vehicle) or 200 ng/well of recombinant BST-2 (rBST-2) for 1 hour. Equivalent concentrations of total proteins from the cells were used to immunoprecipitate BST-2 using anti-BST-2 antibodies (AIDS reagents program) (FIG. 38). Precipitates were separated and blot probed with anti-phospho-tyrosine antibodies (Cell signaling) (FIG. 38). The species-appropriate IRDye secondary antibodies were used followed by detection with the Odyssey Infrared Imaging System (LI-COR Biosciences).

### Results and Conclusion

BST-2-dimerization mutant is not phosphorylated at its cytoplasmic tail (FIG. 38). We conclude that the cytoplasmic tail of BST-2 is phosphorylated upon its activation via BST-2 homodimerization.

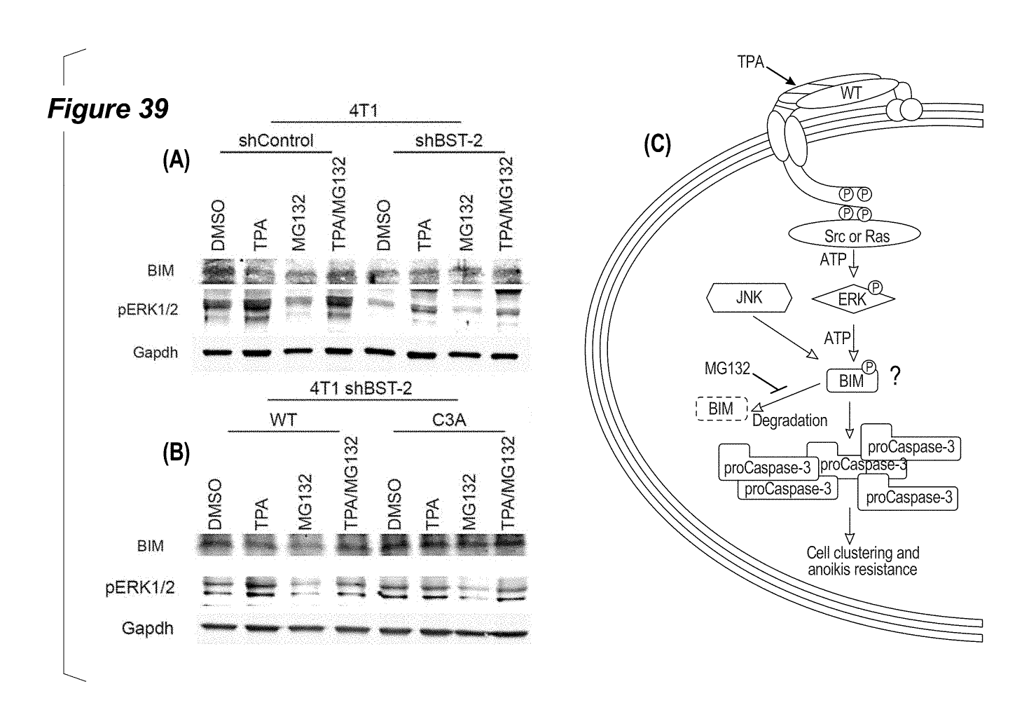

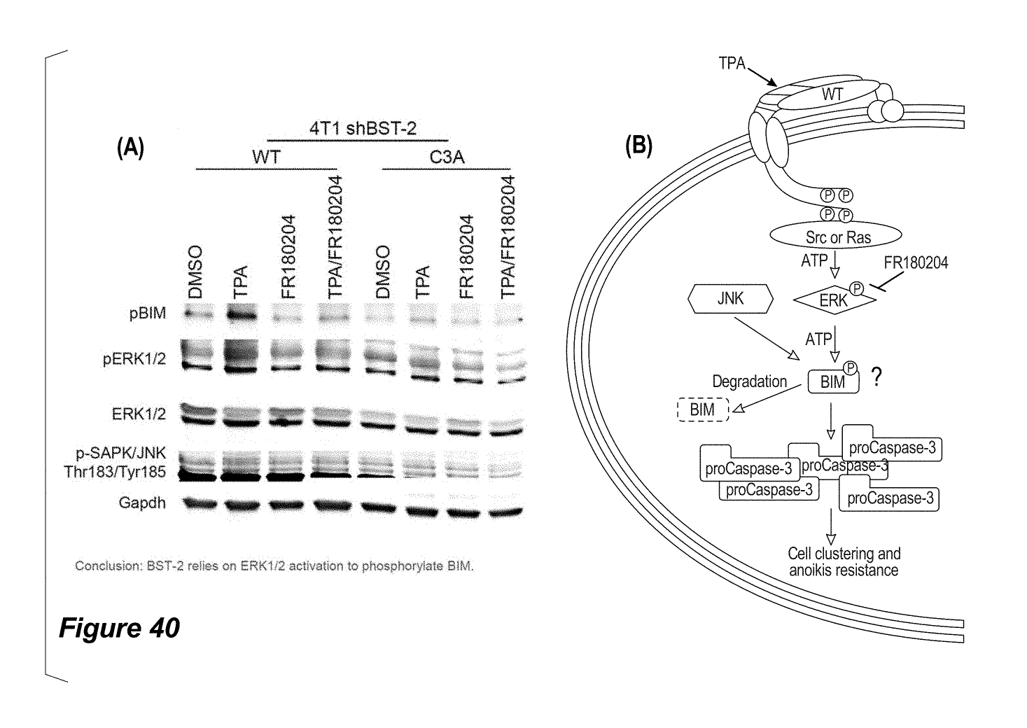

Equivalent numbers (300,000 cells) of sh413 WT and sh413 C3A 4T1 cells were plated on 6-well plates and treated with DMSO (Vehicle), 20 nM of the survival signal TPA (Sigma-Aldrich), 1 uM of the proteasome inhibitor MG132 (Sigma-Aldrich), TPA+MG132, 20 uM of the ERK1/2 kinase inhibitor FR180204 (Sigma-Aldrich) or TPA+FR180204 (TPA/FR180204) for 24 hours following IC₅₀ determination. Equivalent concentrations of total proteins from the cells were separated on a PAGE-gel and probed with anti-cleaved Caspase-3, anti-BIM, and anti-GAPDH antibodies (Santa Cruz Biotechnology) as well as with anti-ERK1/2, anti-pY, anti-pERK1/2, anti-pJNK and anti-pBIM antibodies (Cell Signaling) (FIG. 39 and FIG. 40). The species-appropriate IRDye secondary antibodies were used followed by detection with the Odyssey Infrared Imaging System (LI-COR Biosciences).

### Results and Conclusion

BST-2-dimerization incompenten cells are unable to respond to TPA as a survival signal (FIG. 39). ERK is involved in BST-2-mediated BIM phosphorylation (FIG. 40). We conclude that BST-2 activates ERK1/2 to phosphorylate BIM; a process that leads to proteasomal degradation of BIM and inhibition of anoikis.

### Example 8—Metastasis Studies In Vitro and In Vivo Via Tail Vein

Flow Cytometry.

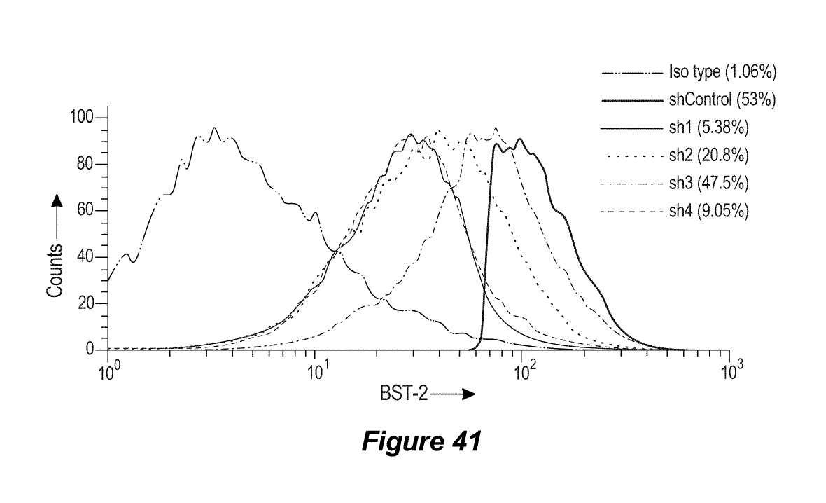

Approximately, 1×10⁶ MDA-MB-231 cells expressing an scramble shRNA (shControl) or shRNAs targeting BST-2 (sh1-4) were stained in PBS with either APC-conjugated anti-human BST-2 (BioLegend) or appropriate immunoglobulin Gs (IgGs) for 1 hour at 4° C. Following washes, cells were stained with 7-AAD (BioLegend) for 15 mins and subjected to FACS. Using FACS calibur flow cytometer (BD), at least 10,000 events were collected per sample. FACS data were analyzed by Flowjo software (TreeStar) (FIG. 41).

3D Migration Assay.

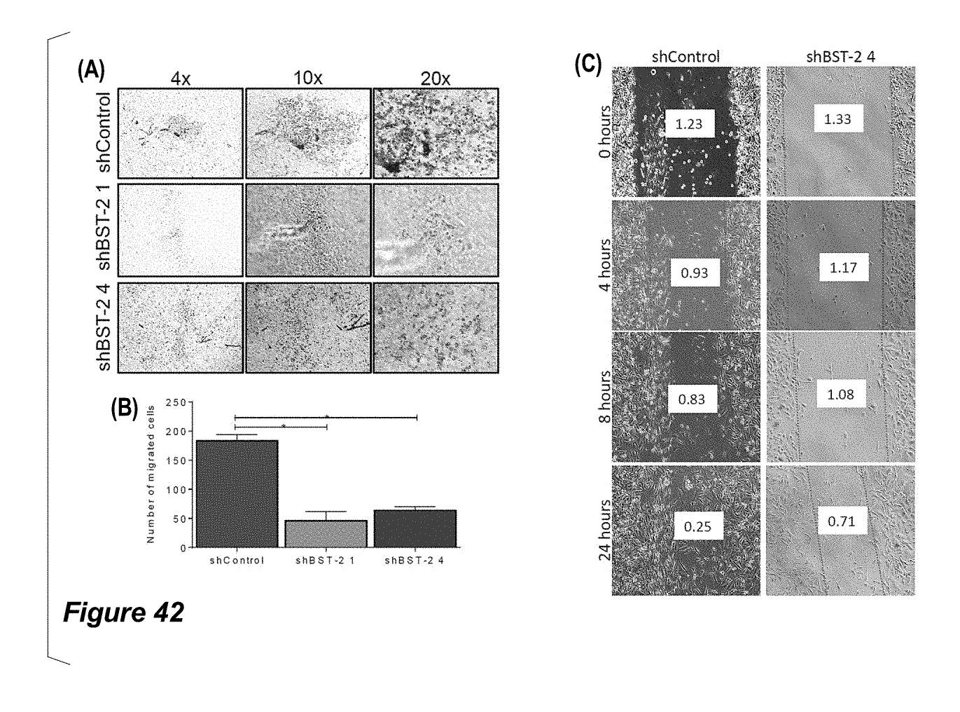

A total of 250,000 MDA-MB-231 cells expressing shControl, sh1, or sh4 were starved for 4 hours and then plated on top of the apical chamber of 24-well cell culture inserts (Merck Millipore). 600 ul of Culture medium containing 30% FBS and 5 μg/ml fibronectin (Sigma-Aldrich) was added to the basal chamber of the unit and cells were allowed to migrate through the insert for 24 hours at 37° C. Cells that did not migrate were washed off; cells that migrated were fixed with 4% PFA, permeabilized with 100% methanol, labeled with Giemsa stain and imaged. Images were processed using ImageJ software. Cells from five different fields were counted and averaged (FIG. 42).

Scratch Assay:

MDA-MB-231 cells expressing shControl or sh4 were plated to confluency on 24-well plates. Cells were scratched and wound closure was assessed at 0, 4, 8 and 24 hours post-scratch. Numbers depict size of wound in relative units (FIG. 42).

Invasion Assay.

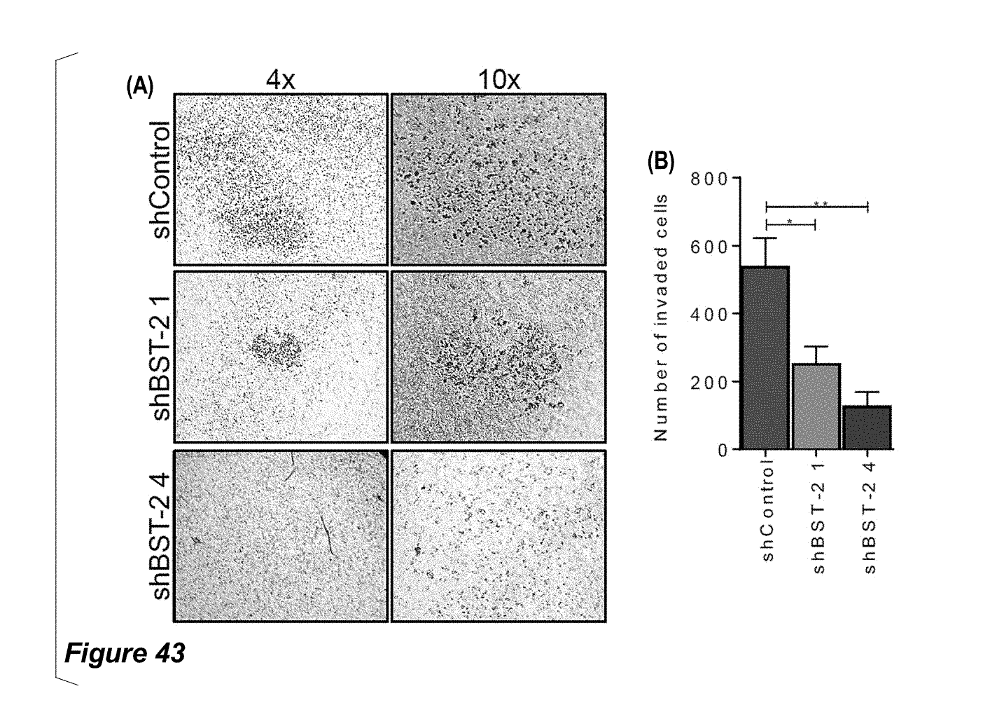

The apical chamber of 24-well cell culture inserts (Merck Millipore) were coated with 1.5 mg/ml of Matrigel (100 μl) (Sigma-Aldrich) and allowed to solidify for 3 hours. A total of 250,000 MDA-MB-231 cells expressing shControl, sh1, or sh4 cells were starved for 4 hours and plated in serum-free medium on top of the Matrigel layer. 600 ul of Culture medium containing 30% FBS and 5 μg/ml fibronectin (Sigma-Aldrich) was added to the basal chamber of the unit and cells were allowed to invade through the membranous barrier for 24 hours at 37° C. Noninvasive cells were washed off; invasive cells were fixed with 4% PFA, permeabilized with 100% methanol, labeled with Giemsa stain and imaged. Images were processed using ImageJ software. Cells from five different fields were counted and averaged (FIG. 43).

Animals.

Five-week-old BALB/cAnNCr female mice were used. Mice were sacrificed when they became moribund. Mouse experiments were approved by the University of Iowa IACUC.

Mice Injections and Live Animal Imaging.

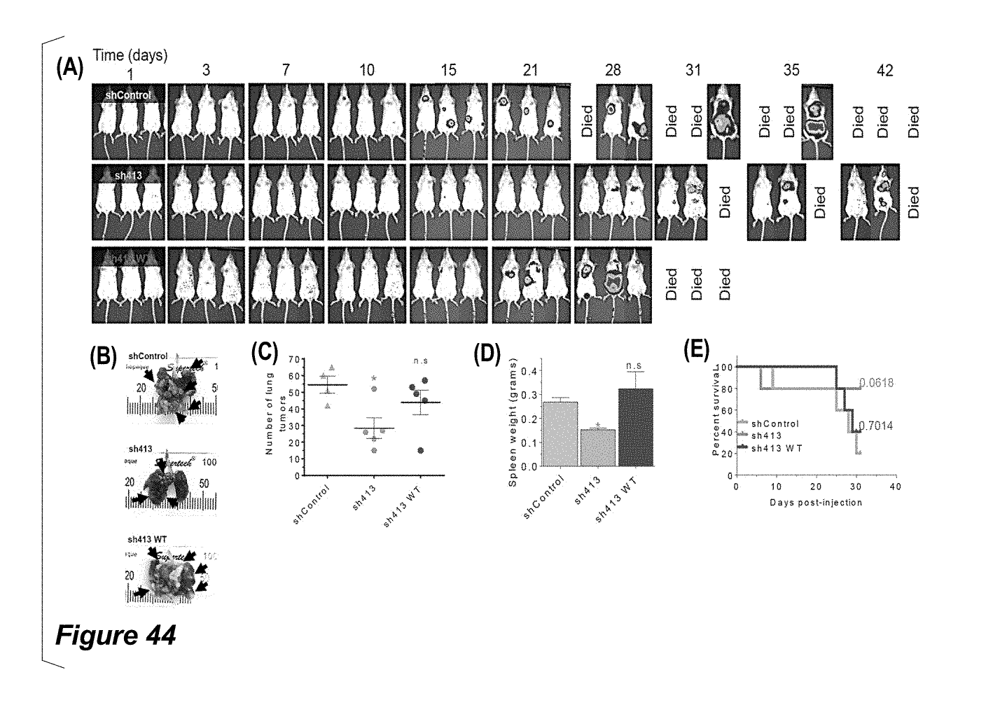

Metastatic tumors were generated by implanting 300,000 4T1 shControl, sh413 or sh413 WT cells in 100 μl of PBS via tail vein in five-week-old female mice. Prior to imaging, mice were anesthetized, weighed and injected intraperitoneally with D-luciferin. Mice were imaged using the Xenogen IVIS three-dimensional optical imaging system (Caliper Life Sciences). Moreover, lung metastatic tumors were imaged post-mortem and counted grossly. Moreover, spleens from shControl, sh413, or sh413 WT injected mice were isolated and weighted. Average splenic weight was calculated from 3 different mice and plotted in a bar graph. Finally, Kaplan-Meier survival plots were generated and analyzed using the Gehan-Breslow-Wilcoxon test (GraphPrism) (FIG. 44).

Reverse Transcriptase Quantitative Real-Time PCR (RT-qPCR).

Isolation of DNA and RNA were accomplished using ZR-Duet DNA/RNA MiniPrep (ZYMO Research) according to manufacturer's instructions. For cDNA synthesis, equivalent amounts of RNA treated with DNase I (Qiagen) were reverse-transcribed with high capacity cDNA reverse transcription Kit (ABI), and the cDNA was amplified with target specific primers. Semi-quantitative PCR was performed using ABI Veriti 96-Well thermal cycler; quantitative real time qPCR (qPCR) and reverse transcription real time qPCR (RT-qPCR) were carried out using ABI 7500 FAST thermal cycler. Primers used: GAPDH-Forward: 5′-CCCCTTCATTGACCTCAACTACA-3′ (SEQ ID NO:6), Reverse: 5′-CGCTCCTGGAGGA TGGTGAT-3′ (SEQ ID NO:7) BIM forward: 5′-ATCGGAGACGAGTTCAACGA-3′ (SEQ ID NO:10), reverse: 5′-TGCC TTCTCCATACCAGA CG-3′ (SEQ ID NO:11); and Caspase-3 forward: 5′-CAAAACCTCAGTGGATT CAAAA-3′ (SEQ ID NO:12), reverse: 5′-CCCATTTCAGGATAATCCATTT-3′ (SEQ ID NO:13).

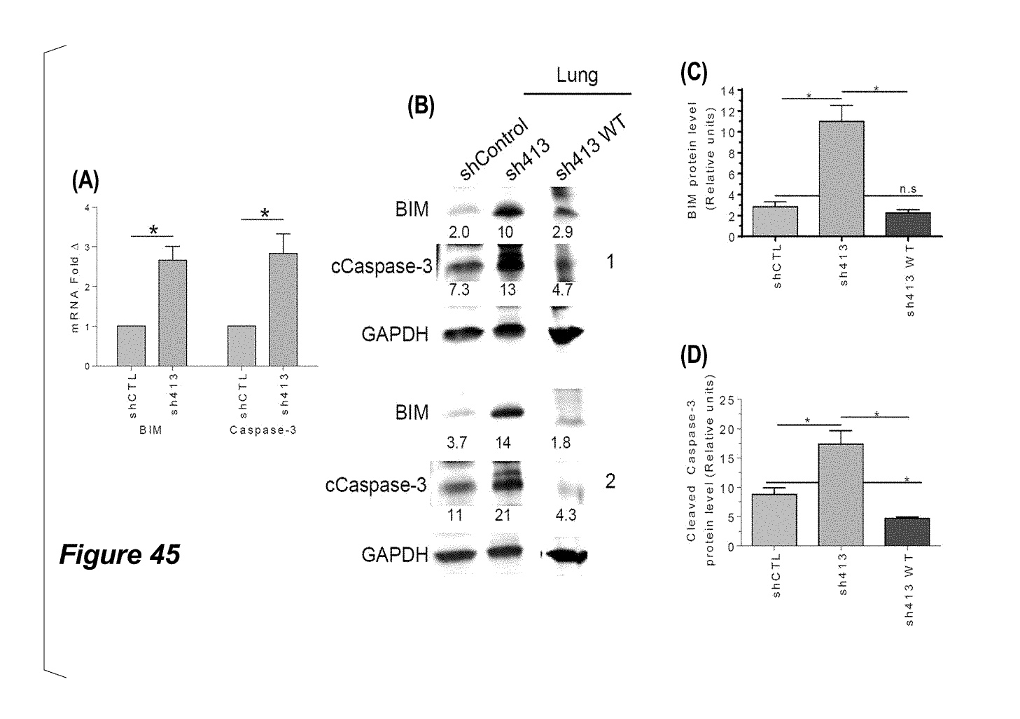

Protein from lungs of mice injected with 4T1 shControl, sh413, or sh413 WT cells via tail vein was isolated to perform western blots blotting for total levels of BIM, levels of activated Caspase-3 (Cleaved Caspase-3) and GAPDH. Bands from 3 different western blots were normalized to GAPDH, quantified, averaged and depicted as bar graphs (FIG. 45).

### Results and Conclusions

The creation of MDA-MB-231 cells that stably express various levels of BST-2 is illustrated in FIG. 41. shControl cells, shBST-2 1 (sh1) cells and shBST-2 4 (sh4) cells were used for in vitro migration and invasion experiments illustrated in FIG. 42 and FIG. 43. FIG. 42 illustrates the effects of BST-2 silencing on migration of Triple Negative Breast Cancer (TNBC) cells. We conclude that migration of TNBC cells, which is a process in metastasis, is dependent on BST-2. FIG. 43 illustrates that BST-2 promotes invasion of MDA-MB-231 TNBC cells In Vitro Effects of BST-2 silencing on invasion of TNBC cells. We conclude that increased BST-2 expression promotes TNBC cell invasion through the extracellular matrix barrier; a process that promotes metastasis. FIG. 44 illustrates that suppression of BST-2 in cancer cells decreases lung metastasis in vivo. We conclude that BST-2 increases the development of pulmonary metastasis of breast cancer cells and subsequent death of tumor-bearing mice. FIG. 45 illustrates that suppression of BST-2 in cancer cells elevates levels of pro-apoptotic factors in the lungs of tumor-bearing mice. We conclude that expression of BST-2 is necessary for downregulation of proapoptotic factors BIM and Caspase-3 at the metastatic sites, resulting in the survival of breast cancer cells.

### Example 9—Studies on B49 Analog: B49Nc

We generated a truncated form of B49 called “B49nc” having the sequence TIKANSEACRDGLRAVMECRNVTHLLQQELTEAQKGFQDVEAQAATCNHTVMA (SEQ ID NO:14). B49nc is a 53 amino acid analog of B49 that exhibits a longer half-life of approximately 20 hours.

Adhesion Assay.

MDA-MB-231 cells expressing shBST-2 or shControl were plated to confluency in a 96-well plate. MDA-MB-231 shControl cells were blocked with water (Vehicle), 200 ng/well of recombinant BST-2 (Sino Biological Inc.), 200 ng/well of B49 peptide or 200 ng of B49nc peptide for 4 hours. Wells were washed twice with PBS and then PKH67 labeled MDA-MB-231 shControl cells were added to wells at 25,000 cells/well. Cells were allowed to adhere for 4 hours at 37° C. Non-adhered cells were washed off with PBS and plates were read at 485 nm/535 nm (excitation/emission) wavelengths using a Tecan Infinite M200 Pro plate reader (Tecan). Values are represented as relative fluorescence intensity (RFI).

### Results and Conclusion

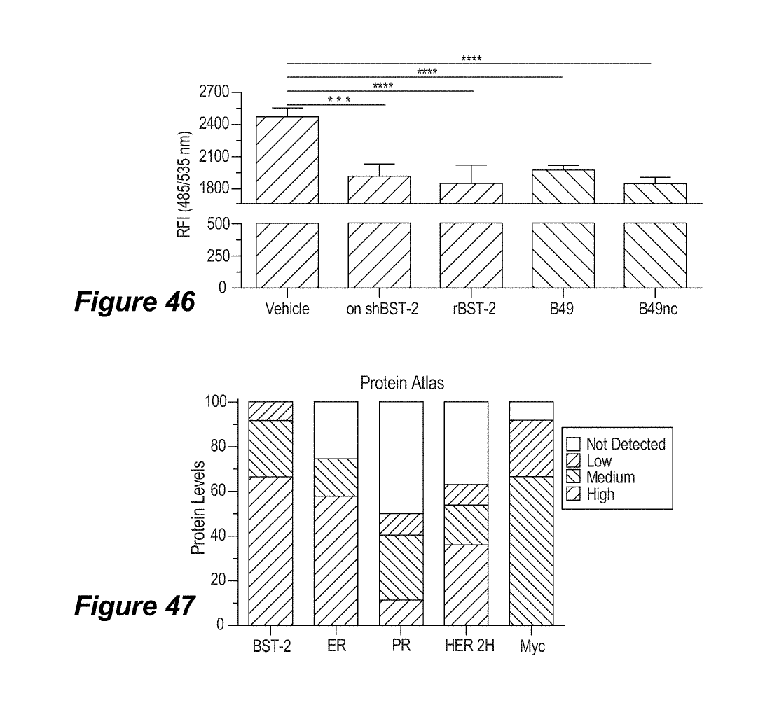

FIG. 46 illustrates that B49nc inhibits cancer cell adhesion similarly as B49. We conclude that disruption of BST-2 dimerization in cancer by B49nc results in decreased cancer cell to cancer cell adhesion which is important for tumor growth.

### Example 10—BST-2 Protein Levels Compared to Other Breast Cancer Targets

BST-2, ER, PR, HER2 and Myc protein levels in tumor tissues from breast cancer patients were extracted from Immunohistochemistry data from proteinatlas.org (http://www.proteinatlas.org/cancer) and plotted as a bar graph (FIG. 47).

### Results and Conclusion

FIG. 47 illustrates the advantages of targeting BST-2 over other current drug targets. We conclude that targeting BST-2 with B49 or B49nc may be a therapy for treating 70% of all breast cancers regardless of their subtype classification or chemotherapy status.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating cancer in a subject in need thereof, wherein the cancer is associated with BST-2 expression or BST-2 biological activity and the method comprises administering a therapeutic agent that inhibits the expression of BST-2 or the biological activity of BST-2, wherein the therapeutic agent is a peptide having a length of 20-80 amino acids and having at least about 80% sequence identity to the amino acid sequence of SEQ ID NO:1

2. The method of claim 1 wherein the subject has breast cancer and the method treats the breast cancer

3. The method of claim 1, wherein the cancer is associated with BST-2 expression and the method comprises administering a therapeutic agent that inhibits the expression of BST-2.

4. The method of claim 1, wherein the cancer is associated with BST-2 biological activity and the method comprises administering a therapeutic agent that inhibits the biological activity of BST-2.

5. The method of claim 4, wherein the therapeutic agent inhibits dimerization of BST-2.

6. (canceled)

7. The method of claim 6, wherein the peptide comprises a contiguous amino sequence of BST-2 of at least about 20 amino acids that binds to full-length BST-2 and inhibits BST-2 from dimerizing.

8. The method of claim 7, wherein the peptide comprises a contiguous amino acid sequence from amino acid 47 to amino acid 95.

9. The method of claim 6, wherein the peptide is conjugated to a reagent that facilitates cell penetration.

10. The method of claim 9, wherein the reagent that facilitates cell penetration is selected from a group consisting of penetratin, TAT, low molecular weight protamine, poly(arginine)₈, nanoparticles, and extracellular vesicles.

11. A pharmaceutical composition comprising a peptide that inhibits dimerization of BST-2 and a carrier, the peptide having a length of 20-80 amino acids and having at least about 80% sequence identity to the amino acid sequence of SEQ ID NO:1.

12. The composition of claim 11, wherein the peptide comprises a contiguous amino sequence of BST-2 of at least about 20 amino acids.

13. The composition of claim 11, wherein the peptide comprises a contiguous amino acid sequence of SEQ ID NO:1 from amino acid 47 to amino acid 95 or an amino acid sequence having at least about 80% sequence identity to SEQ ID NO:1 from amino acid 47 to amino acid 95.

14. The composition of claim 11, wherein the peptide is conjugated to a reagent that facilitates cell penetration.

15. The composition of claim 14, wherein the reagent that facilitates cell penetration is selected from a group consisting of penetratin, TAT, low molecular weight protamine, and poly(arginine)₈, nanoparticles, and extracellular vesicles.

16. A method for diagnosing aggressive, metastatic, and/or triple negative breast cancer in a subject in need thereof, the method comprising detecting expression or biological activity of BST-2 in cells, bodily fluids, or extracellular vesicles.

17. (canceled)

18. The method of claim 1, wherein the peptide comprises the amino acid sequence of SEQ ID NO:14 or an amino acid sequence having at least about 80% sequence identity to SEQ ID NO:14.

19. The method of claim 1, wherein the peptide comprises the amino acid sequence of SEQ ID NO:14

20. The composition of claim 11, wherein the peptide comprises the amino acid sequence of SEQ ID NO:14 or an amino acid sequence having at least about 80% sequence identity to SEQ ID NO:14.

21. The composition of claim 11, wherein the peptide comprises the amino acid sequence of SEQ ID NO:14.
